SEQUENCE LISTING (37 sequences)

SEQ ID NO: 1 - protein, 9 aa, synthetic construct
  MOD_RES 1: note = L-ALANINE (2,3,3,3-D4, 98%)
  MOD_RES 2: note = L-PROLINE (13C5, 99%; 15N, 99%)
  MOD_RES 4: note = L-LEUCINE (D10, 98%)
  MOD_RES 6: note = L-PHENYLALANINE (D8, 98%)
APRLRFYSL

SEQ ID NO: 2 - protein, 9 aa, synthetic construct
  MOD_RES 2: note = L-PROLINE (13C5, 99%; 15N, 99%)
  MOD_RES 4: note = L-LEUCINE (D10, 98%)
  MOD_RES 6: note = L-PHENYLALANINE (D8, 98%)
APRLRFYSL

SEQ ID NO: 3 - protein, 11 aa, synthetic construct
SAPRLRFYSLG

SEQ ID NO: 4 - protein, 11 aa, synthetic construct
GAPRLRFYSLG

SEQ ID NO: 5 - protein, 8 aa, synthetic construct
RGYGYQGL

SEQ ID NO: 6 - protein, 8 aa, synthetic construct
RYPFPGPG

SEQ ID NO: 7 - protein, 9 aa, synthetic construct
APRLRFYSL

SEQ ID NO: 8 - protein, 12 aa, synthetic construct
GRPPGFSFFRGG

SEQ ID NO: 9 - protein, 10 aa, synthetic construct
SAPRLRFYSL

SEQ ID NO: 10 - protein, 10 aa, synthetic construct
GPPGFSPFRG

SEQ ID NO: 11 - protein, 9 aa, synthetic construct
RPPGFSFFR

SEQ ID NO: 12 - protein, 12 aa, synthetic construct
SGLRRASLGGSG

SEQ ID NO: 13 - protein, 13 aa, synthetic construct
SGSLRRASLGGSG

SEQ ID NO: 14 - protein, 9 aa, synthetic construct
  MOD_RES 1: note = L-ALANINE (2,3,3,3-D4, 98%)
  MOD_RES 2: note = L-PROLINE (13C5, 99%; 15N, 99%)
APRLRFYSL

SEQ ID NO: 15 - protein, 14 aa, synthetic construct
GSGSLRRASLGGSG

SEQ ID NO: 16 - protein, 12 aa, synthetic construct
GAPRLRFYSLGG

SEQ ID NO: 17 - protein, 12 aa, synthetic construct
SGPPGFSPFRGS

SEQ ID NO: 18 - protein, 11 aa, synthetic construct
GAPRLRFYSLY

SEQ ID NO: 19 - protein, 7 aa, synthetic construct
LRRASLG

SEQ ID NO: 20 - protein, 17 aa, synthetic construct
GSGSRPPGFSFFRGSGS

SEQ ID NO: 21 - protein, 15 aa, synthetic construct
TSSAPRLRFYSLGSP

SEQ ID NO: 22 - protein, 10 aa, synthetic construct
GPPGPSPFRG

SEQ ID NO: 23 - protein, 8 aa, synthetic construct
RGYAYQGL

SEQ ID NO: 24 - protein, 15 aa, synthetic construct
SGAPRLRFYSLGSGG

SEQ ID NO: 25 - protein, 9 aa, synthetic construct
  MOD_RES 1: note = L-ALANINE (2,3,3,3-D4, 98%)
  MOD_RES 2: note = L-ALANINE (2,3,3,3-D4, 98%)
  MOD_RES 4: note = L-LEUCINE (D10, 98%)
APRLRFYSL

SEQ ID NO: 26 - protein, 8 aa, synthetic construct
SLRRSSLG

SEQ ID NO: 27 - protein, 8 aa, synthetic construct
SFLLRNPS

SEQ ID NO: 28 - protein, 12 aa, synthetic construct
GRPPGFSFFRGG

SEQ ID NO: 29 - protein, 11 aa, synthetic construct
GRPPGFSFFRG

SEQ ID NO: 30 - protein, 9 aa, synthetic construct
PPGPSPFRT

SEQ ID NO: 31 - protein, 15 aa, synthetic construct
SGAPRLRFYSLGSGW

SEQ ID NO: 32 - protein, 13 aa, synthetic construct
YSAPRLRFYSLYQ

SEQ ID NO: 33 - protein, 14 aa, synthetic construct
SGRPPGFSFFRGSG

SEQ ID NO: 34 - protein, 7 aa, synthetic construct
SFYLRNP

SEQ ID NO: 35 - protein, 14 aa, synthetic construct
SSSAPRLRFYSLSY

SEQ ID NO: 36 - protein, 13 aa, synthetic construct
SGRPPGFSFFRGS

SEQ ID NO: 37 - protein, 14 aa, synthetic construct
TSAPRLRFYSLGSY

(19) United States
(12) Patent Application Publication
(10) Pub. No.: US 2025/0369980 A1
(43) Pub. Date: Dec. 4, 2025

(54) MULTIOMIC MASS SPECTROMETRIC IMAGING OF TISSUES USING PHOTOCLEAVABLE MASS-TAG PROBES

(71) Applicant: AmbeGen, Inc., Billerica, MA (US)

(72) Inventors: Mark J Lim, Reading, MA (US); Gargey Yagnik, Hopkinton, MA (US); Jonathan Bell, Billerica, MA (US); Kenneth J. Rothschild, Newton, MA (US)

(21) Appl. No.: 19/216,026

(22) Filed: May 22, 2025

Related U.S. Application Data
(60) Provisional application No. 63/653,139, filed on May 29, 2024.

(51) Int. Cl. G01N 33/68; G01N 1/30; G01N 1/34; G01N 1/36; G01N 1/44; G01N 33/58

(52) U.S. Cl. CPC G01N 33/6848; G01N 1/34; G01N 1/36; G01N 1/44; G01N 33/58; G01N 2001/305; G01N 2458/10

(57) ABSTRACT

The field of this invention relates to immunohistochemistry (IHC), immunocytochemistry and in situ hybridization (ISH) for the targeted detection and mapping of biomolecules (e.g., proteins, glycans and RNAs) in tissues or cells for example, for research use and for clinical use such by pathologists (e.g., biomarker analyses of a resected tumor or tumor biopsy). In particular, the use of mass spectrometric imaging (MSI) as a mode to detect and map the biomolecules in tissues or cells for example. More specifically, the field of this invention relates to photocleavable mass-tag reagents which are attached to Probes such as antibodies and nucleic acids and used to achieve multiplex immunohistochemistry, immunocytochemistry and in situ hybridization, with mass spectrometry (MS) and mass spectrometry imaging (MSI) as the mode of detection/readout.

### CROSS-REFERENCE TO RELATED APPLICATIONS

The present application claims the benefit of U.S. Provisional Patent Application No. 63/653,139, filed on May 29, 2024, which is incorporated herein by reference.

### STATEMENT OF GOVERNMENTAL SUPPORT

This invention was made with government support under grant numbers R44AG078097, R44MH132196, R44CA236097, and R44GM105249 awarded by the NIH. The government has certain rights in the invention.

### SEQUENCE LISTING

A Sequence Listing has been submitted in an XML file named “20695.xml” created on 5/15/2025, consisting of 40,960 bytes, the entire content of which is herein incorporated by reference.

### FIELD OF THE INVENTION

The field of this invention relates to immunohistochemistry (IHC), immunocytochemistry and in situ hybridization (ISH) for the targeted detection and mapping of biomolecules (e.g., proteins, glycans and RNAs) in tissues or cells for example, for research use and for clinical use such by pathologists (e.g., biomarker analyses of a resected tumor or tumor biopsy). In particular, the use of mass spectrometric imaging (MSI) as a mode to detect and map the biomolecules in tissues or cells for example. More specifically, the field of this invention relates to photocleavable mass-tag reagents which are attached to Probes such as antibodies and nucleic acids and used to achieve multiplex immunohistochemistry, immunocytochemistry and in situ hybridization, with mass spectrometry (MS) and mass spectrometry imaging (MSI) as the mode of detection/readout.

### BACKGROUND OF THE INVENTION

A new field of biology and biotechnology known as spatial omics has recently emerged with the goal of spatially imaging in 2D and 3D the large number and varied classes of biomolecules in tissues and cells [1-5]. Spatial omics, which was the 2020 Nature Method of the Year, promises to unravel the immense molecular complexity of tissues at a cellular and subcellular level. This is made possible by imaging methods that are capable of spatially resolving the thousands of biomolecules including different molecular species such as proteins, transcripts, glycans and metabolites which comprise tissues and cells. Immunohistochemistry (IHC), immunocytochemistry (ICC) and in situ hybridization (ISH) are widely used to determine the structural organization of biomolecules at the tissue, cellular and subcellular level [6-8]. For example, IHC is the preferred method for studying extracellular amyloid plaques and intracellular tau-based neurofibrillary tangles in neurodegenerative disorders [9, 10]. In oncology, IHC and ISH can be used to diagnose, classify into subtypes and determine optimal treatment of various cancers [11, 12], including the evaluation of tumor infiltrating lymphocytes (TILs) which are of prognostic value [13]. IHC analyses are generally performed on tissue samples, for example collected by biopsy or surgical resection of a tumor. Typically, tissue samples are fresh frozen (FF) or formalin-fixed and paraffin embedded (FFPE), and then thin-sectioned (e.g., 10 μm) and mounted onto glass microscope slides. In contrast, ICC analyses are generally performed on samples consisting of cells grown on monolayers, cells in suspension deposited on a slide, and cells dissociated from a tissue [14, 15]. Examples include cells from blood plasma such as peripheral blood mononuclear cells (PBMC), bacterial cultures and cells disaggregated from a tissue such as the brain. ISH can be performed on both tissues and cells. Fluorophores or chromogenic agents conjugated to antibody or nucleic acid Probes are the most common methods of visualizing the spatial distribution of targeted biomolecules using microscopy (e.g., protein antigens or genetic material such as miRNA) [8].

It is often vital to simultaneously determine the localization and potential co-localization of a number of biomarkers. This is critical in order to map, for example, the location of the hundreds of possible proteins and/or mRNAs involved in cell regulation and dysregulation in a highly heterogeneous tissue [16, 17]. However, fluorescence microscopy is limited to the simultaneous detection of only a few biomarkers, since molecular fluorophores exhibit relatively broad excitation and emission bands, resulting in spectral overlap [7]. The multiplexing limit of standard fluorescence microscopy is generally 3-5, while hyperspectral/multispectral methods are limited to 8 [7, 18-20]. Furthermore, these multiplexing methods often require cycling strategies (e.g., Perkin Elmer's OPAL multispectral platform) such as iterative staining followed by photobleaching or Probe removal/denaturation [16, 21-23]. Such methods are complex, laborious and incomplete cycling can confound the results [16, 24]. One example is CODEX [25] which is based on iterative fluorescence cycling and has achieved high multiplexity, but suffers from autofluorescence, tissue damage, low dynamic range, and slow image acquisition speeds.

In contrast, mass spectrometric imaging (MSI) facilitates a high level of multiplexing without the limitations of the aforementioned optical methods (limited only by mass resolution which is typically less than 1 Da). Briefly (see FIG. 1 for details), these methods scan the tissue specimen with a mass spectrometer, generating a full mass spectrum at each “pixel” thereby allowing the simultaneous imaging of any given mass species within the spectra [26]. The Caprioli group first introduced this technique based on matrix-assisted laser desorption ionization mass spectrometry (MALDI-MS) [27] which has since been widely adopted for the direct label-free imaging of biomolecules including proteins, nucleic acids, lipids, metabolites and even small drug compounds in complex tissues [28]. This technique has also been extended to other mass spectrometry (MS) approaches such as ESI-based DESI-MS imaging [29]. While MALDI and DESI MSI approaches do not currently match the spatial resolution of optical methods (e.g., 5 μm spatial resolution with the Bruker microGRID accessory for the timTOF fleX instrument, which implements accurate sample stage positioning), it is possible to obtain improved resolution using innovative designs such as transmission geometry (2 μm) [30] or atmospheric pressure MALDI-MSI with laser focusing objectives (1.4 μm) [31]. New approaches to mass spectrometry imaging such as fast mass microscopy (FMM) based on the use of multi-pixel ion detectors promise to produce subμm imaging at orders of magnitude faster image acquisition [32]

However, MSI of intact macromolecules such as proteins is typically not possible due to insufficient mass resolution and poor sensitivity [28]. Identification of a particular biomolecule requires tandem MS/MS fragmentation, ultra-high mass resolution instruments, and/or bottom-up proteomic approaches (e.g., in situ proteolysis of the tissue). For example, for MSI of proteins, researchers typically use a bottom-up approach involving in situ enzymatic digestion of all proteins in the cells/tissue, followed by MSI of the peptide fragments. The multitude of proteins digested in the cell/tissue spot (several thousands), along with the multitude of peptides produced by cell/tissue digestion, coupled with low signal-to-noise, limits sensitivity to only the most abundant proteins (top 5%). Moreover, in order to perform definitive protein identification, these approaches require expensive instruments that can perform liquid chromatography coupled tandem MS/MS [33], procedures which are not amenable to MSI. What is needed is a top-down approach analogous to the universally used immunohistochemistry (IHC) approach which targets specific proteins, but without the limitations on multiplexing.

To overcome this limitation, a few targeted MSI approaches have been introduced which allow multiplex workflows similar to conventional IHC and ISH using labeled antibody and nucleic acid Probes. TAMSIM (targeted multiplex mass spectrometric imaging) is a matrix-free laser desorption ionization (LDI) method which uses antibodies conjugated to small organic photocleavable mass-tags which are cleaved and ionized during MSI [34]. However, the mass-tags are not readily synthesized and only low-plex imaging has been shown [35]. Furthermore, those skilled in the art will recognize that analyte co-crystallization with an excess of exogenously added matrix compound, which facilitates absorption of the mass spectrometer's laser energy and transfer to the analyte, is required for efficient analyte vaporization/ionization and detection, termed matrix-assisted laser desorption ionization (MALDI) mass spectrometry [36, 37], thus, the TAMSIM method will lack sensitivity.

In contrast, peptide mass-tags are easily produced using standard solid-phase synthesis, the masses are readily tuned by altering the sequence, and peptides generally ionize with high efficiency. Lemaire et al. first introduced a photocleavable peptide-based MSI method for targeted imaging of tissue termed Tag-Mass [38]. However, the mass-tagging of the Probe (e.g., antibody) is a complex multi-step process involving an intermediate chemical linker. Moreover, the photocleavable nucleus used in the peptides provides sub-optimal sensitivity. These drawbacks have thus far limited the general utilization of Tag-Mass and consequently only low-plex MSI has been achieved to date [38-40].

Imaging mass cytometry (IMC) uses antibodies tagged with rare earth metals combined with inductively coupled plasma mass spectrometry (ICP-MS) [24]. This approach is capable of achieving approximately 40-plex tissue imaging using the available isotope metal tags for tissue staining. However, this method requires specialized MS instrumentation and is a destructive approach which reduces molecules to elements (atomization) for detection and analysis and is therefore not compatible with performing post-imaging of the tissues using various imaging modalities such as fluorescence imaging or applying a second round of antibody staining with a different panel of antibodies. Such a capability can be used for performing a fast low-resolution scan of an entire specimen followed by higher resolution scans of specific ROIs. See Wilschefski et al. for an example review of ICP-MS [41].

A further drawback of the IC approach is that the Probe labeling process is highly complex, involving pre-loading a polymer with metal ion, partially reducing the antibody and coupling of the two together, with multiple purifications of the polymer and antibody [42].

U.S. Pat. Nos. 11,789,027; 11,906,527; 11,782,056; and 11,846,634 are hereby incorporated by reference. Here, we report novel photocleavable mass-tags (PC-MTs) compositions and MALDI-MSI workflows which overcome these aforementioned limitations. In a preferred embodiment, PC-MTs are modified polypeptides, but virtually any chemical compound detectable by mass spectrometry can serve as a PC-MT. PC-MT antibody and oligonucleotide Probes are produced in a simple and efficient 1- or 2-step reactions. The fast and efficient photo-nucleus [43] used in novel PC-Linkers provides robust sensitivity in practice, allowing high-plex MSI of a wide range of biomarkers in a variety of tissues and cells (see Experimental Examples). Furthermore, novel dual-labeled antibodies, combining both PC-MTs and fluorophores or other detectable moieties such as conjugated DNA, allowed direct correlation of MSI with conventional immunofluorescence or other methods of imaging including ISH based methods. Finally, the versatility of the approach is shown through the ability to perform on the same tissue section both label-free untargeted small molecule MSI (of lipids), not possible by standard IHC, and multiplex PC-MT-based targeted MSI of macromolecular biomarkers (e.g., see U.S. Pat. No. 11,906,527 hereby incorporated by reference).

### SUMMARY OF THE INVENTION

This invention entails compositions as well as methods of production and use of novel Photocleavable Linkers (PC-Linkers), Photocleavable Mass-Tags (PC-MTs) and Photocleavable Mass-Tag Labeled Probes (PC-MT-Probes) which overcome the aforementioned limitations of earlier targeted mass spectrometric imaging (MSI) methods, to enable highly multiplexed MSI of targeted biomolecules in biological specimens such as tissues and cells, using Probes such as antibodies and nucleic acids. PC-MT-Probes are also not limited to mass spectrometric imaging (MSI) but can be used in conjunction with non-imaging mass spectrometry (MS).

The general structure of a PC-MT Labeling Reagent is shown in FIG. 2 (Structure 1) and is comprised of but not limited to the following components: In FIG. 2 (Structure 1) M is a Mass-Tag modified to have a photocleavable terminal amine, W is the Photocleavable Nucleus (PC-Nucleus), V is the Photocleavage Site, X is a Spacer, and Y1 is a Probe-Reactive Moiety. Further details are as follows:

Photocleavable Nucleus (PC-Nucleus) (W in FIG. 2, Structure 1): This is the 1-(2-nitrophenyl)-ethyl based photocleavable nucleus (PC-Nucleus) defined in FIG. 2 (Structure 1) by the chemical structure within the dotted box (W), wherein V is the Photocleavage Site indicated by the black arrow in FIG. 2 (Structure 1). The Photocleavage Site is given in [43], Scheme II.

Mass-Tag Modified to have a Photocleavable Terminal Amine (M in FIG. 2, Structure 1): In a preferred embodiment the Mass-Tag is a peptide or modified peptide chosen due to ease of synthesis (including of different mass species), robust performance in mass spectrometry and the ability to gain further specificity in mass spectrometric identification using established methods of tandem-MS based fragmentation analysis (e.g., MS/MS). It is to be understood for example that any amino acids used in the peptide can be natural or unnatural amino acids as well as modified amino acids, isotopic amino acids, or amino acid analogs/derivatives; or any combination of the aforementioned amino acid types may be used in the peptide. In FIG. 3 (M in Structure 1), an example modified peptide-based Mass-Tag is shown comprising the APRLRFYSL peptide sequence (single-letter code) with an acetyl modified N-terminal alpha-amine (“Ac” in FIG. 3) and a C-terminal modified to have a photocleavable terminal amine. While peptide-based Mass-Tags are one preferred embodiment, the Present Invention is not limited to peptides or any particular chemical composition. The Mass-Tag can for example be any chemical entity which can be detected by mass spectrometry. It is understood by those skilled in the art that an advantage of mass spectrometry is the ability to detect nearly any chemical species, from atoms to compounds, small molecules and macromolecules, and thus nearly any chemical composition can serve as a Mass-Tag.

In one embodiment, a Photocleavable Mass-Tag Precursor can be produced which comprises an amine terminal group attached directly, or indirectly through a linker (Mass Unit Linker), to the Photocleavage Site on the PC-Nucleus (FIG. 4, Structure 1). In this way, any amine reactive (e.g., NHS-activated) Mass Unit can be conjugated to the terminal amine of the Photocleavable Mass-Tag Precursor to create a Mass-Tag of nearly any composition (M in FIG. 4, Structure 2), and therefore the resultant photocleaved Mass Reporter likewise can be of nearly any composition (FIG. 4, Structure 5). Specific examples of how this approach could be used to create Mass-Tags of various compositions are shown in FIG. 5, Structures 5-7, indicated by the M in FIG. 5. The resultant photocleaved Mass Reporters are shown in FIG. 5, Structures 8-10. Polymeric Mass-Tags are preferred due to a general ease of synthesis and the ability to readily modulate the mass by simply altering the number and type of monomeric subunits. While peptides are considered to be a form of biopolymer (in addition to nucleic acids for example), polymers other than peptides may also be used, such as polyethylene glycols (PEGs, e.g., see M in Structure 7 of FIG. 5) which are readily synthesized and detected by MALDI mass spectrometry (MALDI-MS) [44]. However, non-polymeric substances can also serve as Mass-Tags, for example as indicated by the M in Structures 5 and 6 in FIG. 5.

Probe-Reactive Moiety (Y1 in FIG. 2, Structure 1): This is the chemical group used to attach the PC-MT Labeling Reagent to the Probe by chemical reaction (Probes are discussed later). It is to be understood that a variety of Probe Reactive Moieties can be used, including but not limited to amine-reactive N-hydroxysuccinimidyl (NHS) esters (e.g., Y1 in FIG. 3, Structure 1), sulfo-N-hydroxysuccinimidyl (sulfo-NHS) esters, succinimidyl esters (SE), sulfo-succinimidyl esters (SSE), aldehydes, or tetrafluorophenyl (TFP) esters (e.g., Y1 in FIG. 6, Structure 1); sulfhydryl-reactive maleimides (e.g., Y1 in FIG. 7, Structure 1) or iodoacetamides; poly-reactive epoxy moieties; or azides or alkynes (e.g., alkynes such as dibenzocyclooctyne [DBCO] or propargyl groups) such as used in copper-containing or copper-free Click Chemistry [45] (e.g., see Y1 in Structure 1 of FIG. 8 and FIG. 9 for azide examples).

Optional Spacer (X in FIG. 2, Structure 1): The Spacer links the PC-Nucleus (W in FIG. 2, Structure 1) to the Probe-Reactive Moiety (Y1 in FIG. 2, Structure 1). It is to be understood this Spacer is not required, since the Probe-Reactive Moiety can be directly attached to the 1-(2-nitrophenyl)-ethyl based PC-Nucleus at the same position but without the Spacer. It is also to be understood that the Spacer, if present, can be of a variety of chemical structures and that the examples provided are not meant to limit the scope of the Present Invention. The Spacer serves as a bridge between the PC-Nucleus and the Probe-Reactive Moiety and nearly any chemical composition can serve this purpose. For example, the Spacer can simply be a hydrocarbon chain (e.g., aliphatic chain), or alternatively for example, a 2,2′-(ethylenedioxy)-bis-(ethylamine) chemical linker could be used for better solubility in an aqueous environment [46]. Polyethylene glycol (PEG) Spacers are another example and will be recognized by those skilled in the art as excellent chemical linkers which are relatively stable, water soluble and bio-compatible. The Spacer can also be comprised of biopolymers for example, such as peptides and/or nucleic acids. The Spacer can also contain a detectable moiety such as a fluorophore which can serve as an orthogonal detection means (e.g., optical microscopy) whereby the detectable moiety will not be present on the photocleaved Mass Reporter (see Structure 4 of FIG. 2 for the Mass Reporter), and therefore will not impact the mass spectrometric analysis. The Spacer, if present, can be comprised of any combination of the aforementioned structure types, or a stable chemical structure of any kind.

Per FIG. 2, Structures 2-3, the PC-MT Labeling Reagents are attached (conjugated) to Probes or Modified Probes (see P in FIG. 2 for the Probe or Modified Probe). Probes are binding agents which can bind to specific targets in a Sample such as a Biological Sample (e.g., an antibody Probe binding to its target antigen in a tissue Sample for example), whereby Samples include but are not limited to cells and tissues or homogenates thereof, or biological fluids such as blood, serum, or plasma. Probes can be any kind of chemical or biochemical composition. In one preferred embodiment Probes are proteins, including but not limited to antibodies, recombinant antibodies, affibodies, nanobodies, single-chain fragment variable (scFv) antibodies, single domain antibodies, VHH single domain antibodies (e.g., camelid single domain VHH antibodies), receptors, carbohydrate-binding proteins (e.g., lectins [47]), or ligands for example, or fragments thereof. In another preferred embodiment, Probes may also be nucleic acids such as DNA, RNA, or Locked Nucleic Acids (LNA) [48], for example oligonucleotide hybridization Probes or DNA/RNA aptamers. Probes may also be other organic molecules or biomolecules such as lipids, carbohydrates, steroids, or drugs for example.

Probes may have one or more endogenous React-able Groups, for example, primary amines from endogenous lysine amino acids or sulfhydryl groups from endogenous cysteine amino acids in a protein Probe, to which the PC-MT Labeling Reagents may be attached. For example, see P in FIG. 3 for a protein (antibody) Probe comprising endogenous primary amines as the one or more React-able Groups. However, in some cases, it may be necessary to chemically modify the Probe (referred to as a Modified Probe) to create the one or more React-able Groups. Example Modified Probes include but are not limited to alkyne (e.g., DBCO) Modified Probes (e.g., see P in FIG. 9 and FIG. 10) or a Probe (e.g., protein) modified by mild reduction to generate one or more sulfhydryl React-able Groups from disulfide bonds within the protein (e.g., see P in FIG. 7). It is to be understood that these examples are not meant to limit the scope of the chemical composition of the React-able Groups, as a range of endogenous compositions or exogenously added modifications to the Probe can serve as the React-able Group, such as carboxyl groups, phosphates, aldehyde-reactive hydrazides and alkoxyamines, and carbohydrates or carbohydrates modified by mild oxidation to form aldehydes, for example. When React-able Groups are introduced into the Probe by chemical modification, the React-able Group may be accompanied by a chemical linker that serves as a bridge between the React-able Group and the Probe to form the Modified Probe. Like the Spacer (X) in the PC-MT Labeling Reagents already discussed, the chemical linker here on the Modified Probe can be of nearly any chemical composition as previously described.

It is to be understood that while many figures in the Present Invention show attachment of one PC-MT to a Probe or Modified Probe for simplicity, a plurality of PC-MTs can be attached to a Probe or Modified Probe, either directly to the Probe (e.g., directly to an antibody at endogenous sites) or to the modifications on a Probe (e.g., a modified oligo Probe having non-hybridizing nucleic acid sequences [a “Tail” ] further modified to include multiple React-able Groups such as thiols or amines to which a plurality of PC-MTs may be attached).

Probes may have a variety of Targets within a Sample, that is, the molecular structures within the Sample to which the Probes bind. The following examples are not intended to limit the types of Probe Targets: Different Probes may target different biomolecules or biomarkers (e.g., different proteins), or they may target different binding sites within the same biomolecule or biomarker (e.g., different binding sites within the same protein). Probe Targets include but are not limited to biomolecules, or complexes or portions thereof, including proteins, post-translational modifications of proteins, glycoproteins, nucleic acids, lipids and derivatives thereof, drugs, metabolites, carbohydrates, glycans, proteoglycans, gangliosides, glycosaminoglycans (GAGs), and organic compounds.

The PC-MT Labeling Reagent is attached to the Probe or Modified Probe to form the PC-MT Labeled Probe (see the reaction which forms Structure 2 in FIG. 2). The general structure of the PC-MT Labeled Probe is shown as Structure 3 of FIG. 2 and is comprised of but not limited to the following components: In FIG. 2 (Structure 3) M is a Mass-Tag modified to have a photocleavable terminal amine and Z is the Probe Linker which connects the 1-(2-nitrophenyl)-ethyl based Photocleavable Nucleus (PC-Nucleus, W) to the Probe or Modified Probe (P). The Photocleavage Site (V) is also indicated in FIG. 2 (Structure 3). Shown in Structure 2 of FIG. 2, the Probe Linker (Z) is the combination of the Spacer (X) of the PC-MT Labeling Reagent and the chemical composition created by reaction of the Probe-Reactive Moiety (Y1) of the PC-MT Labeling Reagent with the React-able Group on the Probe or Modified Probe, which creates the Reacted Probe-Reactive Moiety (Y2 in Structure 2 of FIG. 2). It should be noted that Structures 2 and 3 in FIG. 2 are chemically identical, wherein Structure 3 merely simplifies the depiction of the combination of the Spacer X and the Reacted Probe-Reactive Moiety (Y2) together as the Probe Linker (Z), as defined in Structure 2 of FIG. 2. Example chemical compositions of the Spacer (X) and Probe-Reactive Moiety (Y1) of the PC-MT Labeling Reagent have already been discussed; hence the Probe Linker (Z) is defined by these compositions following reaction of the Probe-Reactive Moiety (Y1) of the PC-MT Labeling Reagent with the React-able Groups on the Probe or Modified Probe (P). Note also that the reaction of the Probe-Reactive Moiety (Y1) of the PC-MT Labeling Reagent forms a different chemical composition (Y2), as will become apparent in specific Experimental Examples described later.

The PC-MT Labeled Probes are used to bind to and detect molecular targets in samples, such as biological samples including cells and tissues, for example using procedures similar to conventional immunohistochemistry [IHC], immunocytochemistry [ICC], or in situ hybridization [ISH]. When these approaches are used with PC-MT based signal generation and MALDI mass spectrometry readout, they are referred to in the Present Invention as MALDI-IHC, MALDI-ICC and MALDI-ISH. Signal amplification with PC-MT Labeled Probes, including but not limited to using branched DNA (bDNA) based amplification in MALDI-ISH workflows, will be described in the Detailed Description of the Invention.

In another embodiment, the PC-MTs and PC-MT Labeled Probes may be used for encoding and/or detection in microarrays and bead-arrays (e.g., U.S. Pat. Nos. 9,523,680, 9,513,285, and 10,060,912 which are hereby incorporated by reference).

In one embodiment, the present invention contemplates a method of treating a tissue sample, comprising: a) providing i) a mass spectrometric imaging instrument, ii) a source of UV light exogenous to said mass spectrometric imaging instrument, iii) a tissue sample and iv) a first solution of one or more probes conjugated to photocleavable mass-tags; b) treating said tissue sample with said first solution of one or more probes to create a probed tissue sample; c) treating the probed tissue sample with a fixative so that said probes are fixed to said tissue sample; d) illuminating said fixed probes with said exogenous source of UV light so as to photocleave at least a portion of said mass-tags; and e) detecting, using said mass spectrometric imaging instrument, said mass-tags, or fragments thereof as molecular ions, from at least one of said fixed probes.

It is not intended that the present invention be limited by the nature of the mass tag. In one embodiment, the mass tag comprises amino acids, e.g. a plurality of amino acids.

It is not intended that the present invention be limited by the nature of the probe. A variety of probe types are contemplated, including antibody probes, nucleic acid probes, carbohydrate-binding probes and the like. In one embodiment, said first solution comprises a plurality of different probes, each different probe conjugated to a unique photocleavable mass-tag, and at least one of said probes targeting a known biomarker in said tissue sample. In one embodiment, said plurality of different probes are antibodies conjugated to photocleavable mass tags. In one embodiment, said antibodies are in a mixture. In one embodiment, said plurality of different antibody probes are dual-labeled antibody probes, each of said different dual-labeled probes reactive with a different target and conjugated to a photocleavable mass-tag and a detectable label. In one embodiment, said detectable label is an oligonucleotide tag. In one embodiment, the plurality of different antibody probes are fixed to the tissue after step c) to prevent washing out of the antibody in subsequent steps.

It is not intended that the present invention be limited to the type of tissue that is contacted with the various probes. A variety of tissue types are contemplated including but not limited to heart, lung, bone (e.g. bone marrow), ligament, tendon, cartilage, muscle, intestine, kidney, liver, spleen, stomach, prostate, cornea, tongue, tonsil, oral cavity, esophagus, anal canal, lymph node, urethra and vagina and other tissues. These can be cancerous, non-cancerous, or a mixture of both. These can be human tissues or animal tissues. These tissues can contain stem cells, differentiated cells or both.

It is not intended that the present invention be limited to how the tissue is prepared or mounted. In one embodiment, said tissue sample is mounted on a slide. In one embodiment, said slide is coated with Poly-L-lysine to promote adhesion of said tissue sample to said slide. In one embodiment, said tissue sample is formalin-fixed and paraffin-embedded. In one embodiment, said first solution of one or more probes in step b) is directly overlaid onto said tissue sample. In one embodiment, said fixative is paraformaldehyde.

It is not intended that the method of present invention (described above) be limited to only steps a) through e). For example, the method may further comprise a wash step after step b) or a wash step after step c) or both. Wash steps can be performed with a suitable buffer or other reagent. Other steps are also contemplated. In one embodiment, the tissue sample is subjected to a treatment prior to step b), said treatment comprising deparaffinization. In one embodiment, said deparaffinization is performed with xylene. In one embodiment, the tissue sample is further subjected to a treatment, said treatment comprising rehydration. In one embodiment said rehydration is performed with a series of ethanol/water mixtures and aqueous saline buffers. In one embodiment, the tissue sample is further subjected to a treatment, said treatment comprising antigen retrieval. In one embodiment, a matrix compound is applied to said mass-tags before step e). In one embodiment, said tissue sample is treated with a fixative before step b). In one embodiment, said tissue sample is fresh frozen. In one embodiment, the tissue sample is washed after step e) to remove said matrix compound to create a washed tissue sample. In one embodiment, the method further comprises f) contacting said washed tissue sample with a second solution of one or more probes conjugated to photocleavable mass-tags. The second solution can comprise probes that are the same or different from the first solution. For example, the first solution can comprise antibodies to a first set of biomarkers and the second solution can comprise antibodies to a second set of biomarkers.

It is not intended that the present invention be limited by the nature or number of biomarkers. In one embodiment, the present method employs antibodies reactive with CD8a, CD68, Pan-Cytokeratin [PanCK], Ki67, Collagen-1A1 [Col1], and Vimentin [Vim]. These are just examples, as antibodies to other biomarker targets are contemplated (see e.g. Table VI below). In one embodiment, the present method employs a plurality of oligonucleotide probes each of which is designed to bind specifically to a single RNA transcript within a larger transcript population (see e.g. Table V below).

In one embodiment, the present invention contemplates methods where oligonucleotide probes have extra sequences, so-called “tails,” which are not complementary to the target sequence, but which mediate the next hybridization step. In one embodiment, the present invention contemplates a multiplex method for co-detecting a plurality of different nucleic acid target sequences in a tissue sample, said method comprising: a) providing i) a tissue sample comprising different nucleic acid target sequences, ii) transcript-specific oligonucleotide probes comprising sequences complementary to the nucleic acid target sequences and first tail sequences not complementary to the target sequence; iii) pre-amplifier oligonucleotides comprising sequences complementary to the first tail sequence on the transcript-specific oligonucleotide probe and a second tail sequences not complementary to the target sequence; and detector probes with sequences complementary to said second tail sequences, each detector probe conjugated to a unique photocleavable mass-tag; b) contacting said tissue sample with said transcript-specific oligonucleotide probes to create a probed tissue sample; c) washing said tissue sample to remove excess transcript-specific oligonucleotide probes; d) contacting said probed tissue sample with said pre-amplifier oligonucleotides, such that at least a portion hybridize with said first tail sequences so as to create a transcript-specific oligonucleotide probe preamplifier oligonucleotide probe constructs; e) contacting said constructs with said detector probes so as to create bound detector probes; f) illuminating said photocleavable mass-tags of said bound detector probes with an exogeneous source of UV light so as to photocleave at least a portion of said mass-tags before step g); and g) detecting, using mass spectrometric imaging of said probed tissue sample, said unique mass-tags, or fragments thereof, from said bound detector probes, wherein said mass-tags are detected as molecular ions.

In an alternative embodiment, the present invention contemplates a multiplex method for co-detecting a plurality of different nucleic acid target sequences in a tissue sample, said method comprising: a) providing i) a tissue sample comprising different nucleic acid target sequences, ii) transcript-specific oligonucleotide probes comprising sequences complementary to the nucleic acid target sequences and first tail sequences not complementary to the target sequence; iii) pre-amplifier oligonucleotides comprising sequences complementary to the first tail sequence on the transcript-specific oligonucleotide probe and a second tail sequences not complementary to the target sequence; and detector probes with sequences complementary to said second tail sequences, each detector probe conjugated to a unique photocleavable mass-tag; b) contacting said tissue sample with said transcript-specific oligonucleotide probes to create a probed tissue sample; c) washing said tissue sample to remove excess transcript-specific oligonucleotide probes; d) contacting said probed tissue sample with said pre-amplifier oligonucleotides and detector probes, such that at least a portion of the pre-amplifier oligonucleotides hybridize with the tails of said transcript-specific oligonucleotide probes and at least a portion of detector probes hybridize with the tails of said pre-amplifier probes; e) illuminating said photocleavable mass-tags of said bound detector probes with an exogeneous source of UV light so as to photocleave at least a portion of said mass-tags before step f); and f) detecting, using mass spectrometric imaging of said probed tissue sample, said unique mass-tags, or fragments thereof, from said bound detector probes, wherein said mass-tags are detected as molecular ions.

In another embodiment, the present invention contemplates a method which is not strictly branched but still involves hybridization of a series of hybridization probes which contain tails. In this case it is linear and occurs all at once (referred to as hybridization complex reaction-HCR). This involves first i) an initiator sequence complementary to the target DNA and contains a tail and ii) a series of two amplifiers which each have tails with the first complementary to the tail of the initiator and the second complementary to the tail of the first initiator. The tail of the second is complementary to the same tail sequence as the tail on the initiator so the complex forms a linear sequence. In this case the PCMTs are attached to the ends of each of the initiator sequences so in a sense each amplifier is also a detector probe. All these reactions occur in the same test tube and occur spontaneously.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Conventional Bottom-Up Application of Mass Spectrometric Imaging (MSI) for Multiplex Direct Label-Free Mapping of Analytes in Tissue Samples. Adapted from [26]. This existing approach is multiplexed but inherently untargeted as it does not use labeled Probes such as photocleavable mass-tagged labeled Probes (PC-MT Labeled Probes) and therefore requires complex methods of identification of the analytes detected, which in some cases also degrades the spatial resolution of the image (e.g., since in situ tissue proteolysis is required for protein detection and identification).

FIG. 2. General Structure of Photocleavable Mass-Tag (PC-MT) Labeling Reagents (Structure 1) and Photocleavable Mass-Tag (PC-MT) Labeled Probes (Structure 3). Wherein M is a Mass-Tag modified to have a photocleavable terminal amine; W is the Photocleavable Nucleus (PC-Nucleus); V is the Photocleavage Site; X is a Spacer; Y1 is a Probe-Reactive Moiety; P is a Probe or Modified Probe comprising one or more React-able Groups; and Y2 is the Reacted Probe-Reactive Moiety, wherein X and Y2 together are Z, the Probe Linker. The “Mass Reporter” is what is detected/analyzed in a mass spectrometer after photocleavage.

FIG. 3. Example Structure of an NHS-ester Based Photocleavable Mass-Tag (PC-MT) Labeling Reagent (Structure 1) and Resultant Photocleavable Mass-Tag (PC-MT) Labeled Probe (Structure 3). Wherein M is a Mass-Tag modified to have a photocleavable terminal amine; V is the Photocleavage Site; X is a Spacer; Y1 is a Probe-Reactive Moiety; P is a Probe or Modified Probe comprising one or more React-able Groups; and Y2 is the Reacted Probe-Reactive Moiety, wherein X and Y2 together are Z, the Probe Linker. The “Mass Reporter” is what is detected/analyzed in a mass spectrometer after photocleavage. The central structures are derived from the Fmoc-PC-Linker shown in FIG. 13 which is used to produce the PC-MT Labeling Reagent by solid-phase peptide synthesis (SPPS).

FIG. 4. General Structure of Photocleavable Mass-Tag (PC-MT) Precursors (Structure 1), and Resultant Photocleavable Mass-Tag (PC-MT) Labeling Reagents (Structure 2) and Photocleavable Mass-Tag (PC-MT) Labeled Probes (Structure 3). Wherein L is a Mass Unit Linker; X is a Spacer; Y1 is a Probe-Reactive Moiety; U is a Mass Unit, together with its chemically reactive group (e.g., NHS-ester shown) comprises the “Reactive Mass Unit” prior to reaction with the PC-MT Precursor; M is a Mass-Tag modified to have a photocleavable terminal amine; P is a Probe or Modified Probe comprising one or more React-able Groups; and Y2 is the reacted Probe-Reactive Moiety, wherein X and Y2 together are Z, the Probe Linker. The “Mass Reporter” is what is detected/analyzed in a mass spectrometer after photocleavage.

FIG. 5. Example Structure of a Photocleavable Mass-Tag (PC-MT) Precursor (Structure 1) and Resultant Azide Based Photocleavable Mass-Tag (PC-MT) Labeling Reagents (Structures 5-7). Wherein L is a Mass Unit Linker; X is a Spacer; Y1 is a Probe-Reactive Moiety; U indicates example Mass Units, together with their chemically reactive groups (e.g., NHS-esters and TFP-esters shown) comprise the “Reactive Mass Units” prior to reaction with the PC-MT Precursor; M are Mass-Tags modified to have a photocleavable terminal amine; The “Mass Reporters” (Structures 8-10) are what are detected/analyzed in a mass spectrometer after attachment to Probes or Modified Probes and photocleavage. The central structures are derived from the Fmoc-PC-Linker shown in FIG. 13 which is used to produce the PC-MT Precursor by solid-phase peptide synthesis (SPPS); in this case the Fmoc-PC-Linker is the last “amino acid” (amino acid analog) added in the SPPS.

FIG. 6. Example Structure of a TFP-ester Based Photocleavable Mass-Tag (PC-MT) Labeling Reagent (Structure 1) and Resultant Photocleavable Mass-Tag (PC-MT) Labeled Probe (Structure 3). Wherein M is a Mass-Tag modified to have a photocleavable terminal amine; V is the Photocleavage Site; X is a Spacer; Y1 is a Probe-Reactive Moiety; P is a Probe or Modified Probe comprising one or more React-able Groups; and Y2 is the Reacted Probe-Reactive Moiety, wherein X and Y2 together are Z, the Probe Linker. The “Mass Reporter” is what is detected/analyzed in a mass spectrometer after photocleavage.

FIG. 7. Example Structure of a Maleimide Based Photocleavable Mass-Tag (PC-MT) Labeling Reagent (Structure 1) and Resultant Photocleavable Mass-Tag (PC-MT) Labeled Probe (Structure 3). Wherein M is a Mass-Tag modified to have a photocleavable terminal amine; V is the Photocleavage Site; X is a Spacer; Y1 is a Probe-Reactive Moiety; P is a Probe or Modified Probe comprising one or more React-able Groups; and Y2 is the Reacted Probe-Reactive Moiety, wherein X and Y2 together are Z, the Probe Linker. The “Mass Reporter” is what is detected/analyzed in a mass spectrometer after photocleavage.

FIG. 8. Example Structure of an Azide Based Photocleavable Mass-Tag (PC-MT) Labeling Reagent (Structure 1) and Resultant Photocleavable Mass-Tag (PC-MT) Labeled Antibody Probe (Structure 3). Wherein M is a Mass-Tag modified to have a photocleavable terminal amine; V is the Photocleavage Site; X is a Spacer; Y1 is a Probe-Reactive Moiety; P is a Probe or Modified Probe comprising one or more React-able Groups; and Y2 is the Reacted Probe-Reactive Moiety, wherein X and Y2 together are Z, the Probe Linker. The “Mass Reporter” is what is detected/analyzed in a mass spectrometer after photocleavage.

FIG. 9. Example Structure of an Azide Based Photocleavable Mass-Tag (PC-MT) Labeling Reagent (Structure 1) and Resultant Photocleavable Mass-Tag (PC-MT) Labeled Oligo Probe (Structure 3). Wherein M is a Mass-Tag modified to have a photocleavable terminal amine; V is the Photocleavage Site; X is a Spacer; Y1 is a Probe-Reactive Moiety; P is a Probe or Modified Probe comprising one or more React-able Groups; and Y2 is the Reacted Probe-Reactive Moiety, wherein X and Y2 together are Z, the Probe Linker. The “Mass Reporter” is what is detected/analyzed in a mass spectrometer after photocleavage.

FIG. 10. Example PC-MT Signal Amplification using Mass-Tag Carriers which Each Attach to 1 Site on the Probe or Modified Probe and Each Yielding Multiple PC-MT Attachment Sites (React-able Groups). Wherein M is a Mass-Tag modified to have a photocleavable terminal amine; V is the Photocleavage Site; X is a Spacer; Y1 is a Probe-Reactive Moiety; P is a Probe or Modified Probe comprising one or more React-able Groups; and Y2 is the Reacted Probe-Reactive Moiety. Note that the PC-MT Labeling Reagent (Structure 2) has been simplified to Structure 2.1 for the sake of easier viewing in Structure 3 (PC-MT Labeled Probe). It is to be understood that while each Mass-Tag Carrier attaches to a single site on the Probe or Modified Probe, one or more Mass-Tag Carriers (one depicted) may be attached to a Probe or Modified Probe.

FIG. 11. More Examples of PC-MT Signal Amplification using Mass-Tag Carriers which Each Attach to 1 Site on the Probe or Modified Probe and Each Yielding Multiple PC-MT Attachment Sites (React-able Groups). In this case Antibody Probes are shown conjugated to Mass-Tag Carriers that comprise 3 (Structure 1) and 7 (Structure 2) available React-able Groups (alkynes; indicated with asterisks) for PC-MT attachment (e.g., using azide-based PC-MT Labeling Reagents and Click Chemistry). PC-MTs are not shown in this figure. It is to be understood that while each Mass-Tag Carrier attaches to a single site on the Probe or Modified Probe, one or more Mass-Tag Carriers (one depicted) may be attached to a Probe or Modified Probe.

FIG. 12. Comparison of essential common elements of the protocol for the Present Invention (MALDI-IHC and MALDI-ISH) to that of conventional IHC (e.g., [8]) and conventional ISH (e.g., [17]), as well as to conventional direct MSI (e.g., [27]) and to MSI of bead-arrays (e.g., U.S. Pat. No. 9,523,680 which is hereby incorporated by reference). Note that many protocol variations are possible for example depending on whether FF or FFPE tissues are used, whether it is IHC/MALDI-IHC or ISH/MALDI-ISH based protocol, and/or what types of optical detection methods are used for conventional IHC or ISH (such as directly labeled primary antibodies or secondary detection methods; and colorimetric versus fluorescence readout), therefore FIG. 12 shows only common essential elements of the protocols.

FIG. 13. Example Photocleavable Linker (PC-Linker) used in the Synthesis of PC-MT Labeling Reagents. The right section structures indicate the PC-Linker portion, while the remainder is the Fmoc protecting group necessary for use of this reagent in conventional solid-phase peptide synthesis (SPPS).

FIG. 14A-C shows MALDI-IHC With and Without Antibody Post-Fixation (see also Example 1). FIG. 14A shows an example MALDI-IHC image (without Post-Fixation) of a human tonsil tissue section with the 6 Mass Reporters from the 6 PC-MT-antibodies used to “stain” the tissue colorized according to the key provided in the image. A diagrammatic representation of a PC-MT-antibody is also shown. FIG. 14B shows an example MALDI-IHC images (with and without PFA-based Post-Fixation) of a human tonsil tissue section with 4 Mass Reporters from 4 PC-MT-antibodies used to “stain” the tissue colorized according to the key provided in the image.

FIG. 14C shows a Graph of Mass Reporter intensities of the 4 PC-MT-antibodies averaged over the entire tissue section, comparing with and without PFA-based Post-Fixation.

FIG. 15. MALDI-IHC using PC-MT-Antibodies Prepared using a TFP-ester Based PC-MT Labeling Reagent (see also Example 2). A PC-MT-antibody to the CD20 B-cell marker was prepared using a PC-MT-Labeling Reagent which comprised an amine-reactive TFP-ester based Probe-Reactive Moiety. The PC-MT-antibody was used in MALDI-IHC staining and imaging of human tonsil tissue, with the MALDI-IHC image of CD20 shown.

FIG. 16A-K shows Multiplexed MALDI-ISH on Fresh Frozen Tissue Sections (see also Example 4). FIG. 16A shows that PC-MT-oligos were prepared and analyzed by agarose gel electrophoresis at various stages in the process as indicated by the lane key shown in the figure.

FIG. 16B shows the PC-MT-oligos were used to perform multiplexed MALDI-ISH on hABeta^{SAA} fresh frozen Alzheimer's mouse brain tissue sections. A MALDI-ISH image is shown for some of the targets in the multiplexed experiment. The Transcript-Specific Z-Probes bind the mouse brain targets listed in Table IV, whereas the Negative Control Z-Probes target bacterial sequences which do not exist in the tissue. FIG. 16C-J shows Single Mass Reporter images for 8 of the targets in the multiplexed experiment. FIG. 16K shows overall mean mass spectrum of the tissue section stained with the Transcript-Specific Z-Probes, with the Mass Reporter monoisotopic peaks for 8 of the targets labeled accordingly.

FIG. 17A-I. Multiplexed MALDI-ISH on FFPE Tissue Sections (see also Example 5). (FIG. 17A) PC-MT-oligos were used to perform multiplexed MALDI-ISH on hABeta^{SAA} FFPE Alzheimer's mouse brain tissue sections. A MALDI-ISH image is shown for some of the targets in the multiplexed experiment. The Transcript-Specific Z-Probes bind the mouse brain targets listed in Table IV, whereas the Negative Control Z-Probes target bacterial sequences which do not exist in the tissue. (FIG. 17B-I) Single Mass Reporter images for 8 of the targets in the multiplexed experiment.

FIG. 18. Tri-Omic MALDI-MSI on the Same Fresh Frozen Tissue Section (see also Example 6). An hABeta^{SAA} fresh frozen (FF) Alzheimer's mouse brain tissue section was used. MALDI-MSI images from the same tissue section are shown for untargeted label-free endogenous lipid imaging (“lipidomics”), MALDI-ISH (“transcriptomics”) and MALDI-IHC (“proteomics”). The molecular species imaged are colorized according to the keys provided in the images (note that lipids are listed only as m/z mass assignments since conclusive identification would require further analyses such as LC-MS/MS fragmentation analysis).

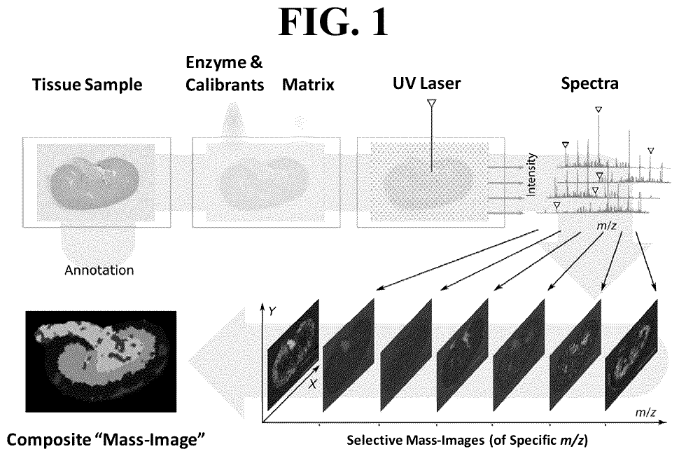

### DETAILED DESCRIPTION OF THE INVENTION

Signal Amplification with PC-MT Labeled Probes

The amplification of signal using PC-MTs and Probes can be accomplished by using various methods where the linkage between the PC-MT and Probe is non-covalent and accomplished through the use of DNA hybridization. Such methods have been employed previously to amplify fluorescent signals from Probes including but not limited to oligonucleotide and antibody Probes. For example, in the case of fluorescence in situ hybridization (FISH), which reveals the abundance and position of nucleic acid sequences in fixed samples such as FFPE tissue, the fluorescence signal originating from oligonucleotide hybridization Probes that bind to target DNA or RNA sequences can be amplified with such methods. One example if SABER-FISH [49] which uses oligonucleotide-based FISH Probes which are extended with multiple single stranded DNA concatemer sequences which are added to the hybridization Probe using primer-exchange reactions (PER). The concatemer sequences are labeled by using fluorescent imager molecules which have complementary sequences to the concatemers allowing secondary hybridization to occur under well controlled conditions. The ability of multiple fluorescent imager molecules to bind to the hybridization Probe produces amplification of the fluorescence signal which in the case of Kishi et al. was determined to range between 5-450-fold in fixed cells and tissues. Since different concatemer sequences can be programed for specific hybridization Probes and different complementary sequences used for the individual fluorescent imager molecules, high multiplexing can be achieved using SABER-FISH.

A similar approach can be used in the case of PC-MTs where the imager Probe consists of an oligonucleotide (oligo) sequence linked to a PC-MT to create a PC-MT Labeled Oligo Probe (PC-MT-oligo; e.g., as made in Example 4).

Importantly, PC-MT-oligo Probes can be used not only to amplify signals from DNA hybridization Probes in conjunction with MALDI-ISH as described above, but also in the case of antibodies or other proteinaceous Probes used for IHC in conjunction with MALDI-IHC. In the case of antibody and other proteinaceous Probes, the Probes are conjugated with an oligo coding molecule with a predetermined sequence (e.g., DNA or RNA) which acts very similar to the target oligo molecules used in FISH, where a hybridization Probe is used for detection of the coding sequence. Methods for conjugating DNA to antibodies and other proteins are well known in the literature and to those skilled in the art and can be performed using commercial kits or through commercial services. The sequence of the oligo molecule that is conjugated to the antibody or other protein is designed to avoid non-specific binding of the complementary hybridization Probe to endogenous DNA/RNA in the tissue and designed for hybridization to be performed at a specific temperature range. One example where antibodies are conjugated to coding DNA is for CODEX, where the antibody is imaged in a tissue using a series of hybridization Probes that are conjugated with fluorophores. For the purpose of amplification, the hybridization Probe contains not only complementary sequences to the coding DNA on the antibody or other protein but also sequences that can be used similar to SABER-FISH such as concatemers that will allow multiple PC-MT-oligos to bind. This approach also allows for repeated cycles of hybridization Probes.

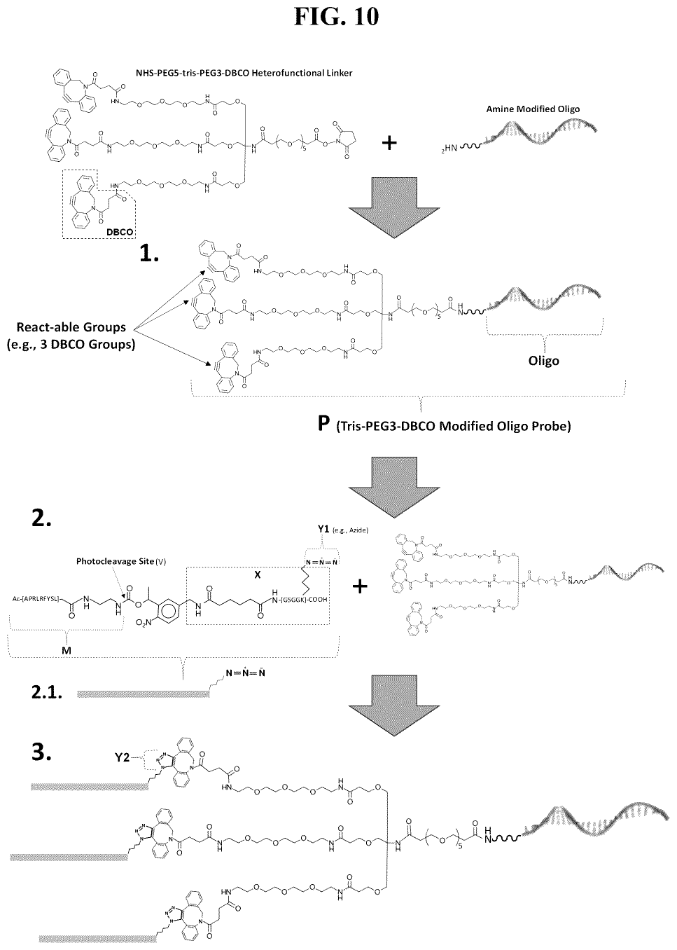

In another embodiment, PC-MT signal amplification can be achieved by direct covalent conjugation of Mass-Tag Carriers (e.g., branched polymers) to the Probe or Modified Probe which carry multiple PC-MT chemical attachment sites (React-able Groups), such that while each Mass-Tag Carrier is attached to only one site on the Probe or Modified Probe, each Mass-Tag Carrier can be conjugated to more than one PC-MTs. FIG. 10 shows one example of attaching an NHS-activated branched PEG molecule having 3 DBCO groups (CONJU-PROBE Catalog CP-2235) as the Mass-Tag Carrier to the 5′ terminal amine of a modified oligo Probe. The 3 DBCO React-able Groups allow attachment of 3 PC-MTs by Click Chemistry using azide Probe-Reactive Moieties on the PC-MT Labeling Reagent (FIG. 10, Structures 2-3).

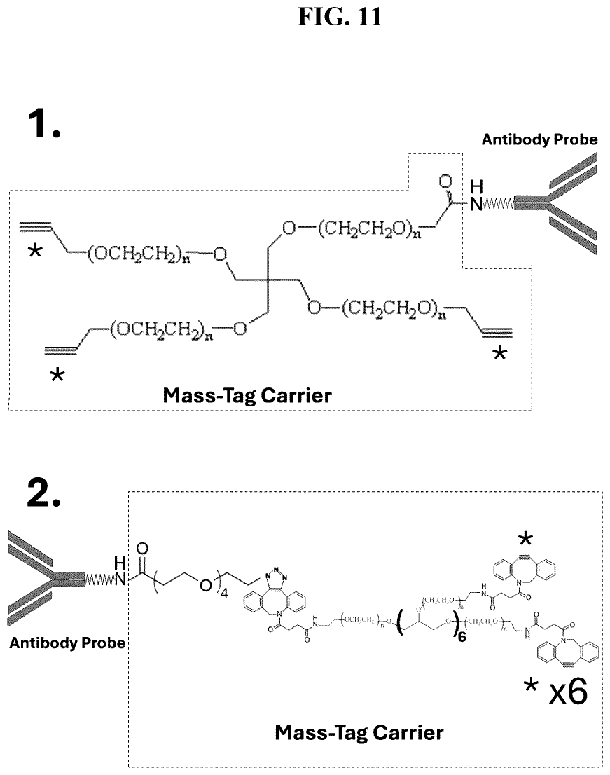

FIG. 11 shows more examples of this Mass-Tag Carrier amplification approach. In FIG. 11 Structure 1, an NHS-activated branched PEG carrying 3 alkyne groups (Creative PEGWorks Catalog CPW-4412) is attached to an antibody Probe as the Mass-Tag Carrier. Alternatively, in FIG. 11 Structure 2, an NHS-activated linear compound carrying only 1 azide is first attached to an antibody Probe (NHS-PEG4-Azide; Thermo Scientific Catalog 26130) to create an azide Modified Probe, followed by attachment of a branched 8-arm PEG Mass-Tag Carrier having 8 DBCO groups (Creative PEGWorks Catalog PSB-8072), with one DBCO group consumed in this attachment to the Modified Probe. In both aforementioned cases, PC-MTs can be attached to the Mass-Tag Carriers using PC-MT Labeling Reagents having an azide Probe-Reactive Moiety, through the use of Click Chemistry (copper-mediated Click Chemistry for the Probe composition in FIG. 11 Structure 1, and copper-free Click Chemistry for the Probe composition in FIG. 11 Structure 2).

It is to be understood that while each Mass-Tag Carrier attaches to a single site on the Probe or Modified Probe, one or more Mass-Tag Carriers may be attached to a Probe or Modified Probe.

Treating Tissues with PC-MT Labeled Probes and Mass Spectrometry Imaging (MSI)

Biological samples such as cells and tissues mounted on a substrate such as a microscope slide (with or without a conductive coating), are typical samples in the Present Invention. However, the Present Invention is not intended to be limited to tissues as the sample. For example, the compositions and methods described in the Present Invention can be applied to cells grown or deposited on surface or biofilms grown or deposited on surface. For example, there has been rapid growth in the use of MSI for rapid identification of microorganisms in clinical microbiology [50, 51]. Bacterial cells are grown or deposited on substrates and then MALDI-MSI is performed. In an additional example, cells derived from a cancer biopsy and deposited on a surface can be analyzed using the compositions and methods described in the Present Invention. An additional example is bacteria both of a single species or multiple species grown on a surface to form a complex heterogeneous pattern. An additional example is the MALDI-MSI of biofilms. Recent progress has been made in applying this method to Bacillus subtilis biofilms grown on agar by using a sprayer to deposit specific matrix compounds compatible with MALDI-MSI such as 2,5-dihydroxybenzoin acid solutions [52]. The method could also be applied to complex multicellular whole organisms deposited on a surface. For example, C. elegans is a free-living, transparent nematode, about 1 mm in length, that lives in temperate soil environments. MSI has been previously applied to C. elegans demonstrating the feasibility of applying the compositions and methods described in the Present Invention [53]. The compositions and methods described in the Present Invention can also be applied to subcellular or molecular assemblies that are grown or deposited on a surface including both organic and nonorganic nanostructures. Examples of MSI profiling of single cells and subcellular structures are described in a recent review by Lanni et al. [54]. Recently, subcellular resolution has been obtained with MALDI-MSI by using specialized techniques such as transmission-mode geometry [55] that are compatible with the compositions and methods described in the Present Invention.

Another example is Peripheral blood mononuclear cell (PBMC) suspensions which comprise a highly heterogeneous population of cell types that have direct relevance to clinical diagnostics of a range of diseases including hematological diseases and cancer. Subfractions of cells from other biological fluids such as cerebral spinal fluid (CSF), saliva, and urine might also be subjected to the methods of the Present Invention. One major use of the compositions and methods of the Present Invention is in the area of non-invasive liquid biopsies (LBs) for cancer which have attracted widespread interest in the past two decades due to their many potential advantages [56-60]. Not only because LBs hold significant promise for clinical applications [57, 61, 62] but also because they have potential for understanding the biological mechanisms of metastatic cancer. Specific advantages include early detection, lower risk of complications, real-time cancer monitoring, lower costs [63], and potential for comprehensive molecular profiling that reflects the clonal heterogeneity of tumors [59].

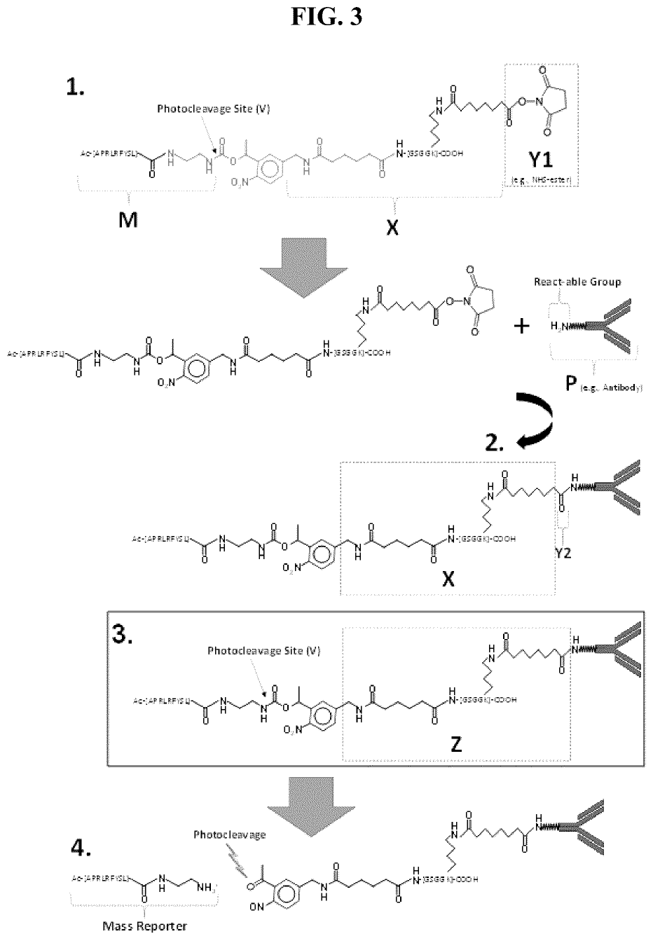

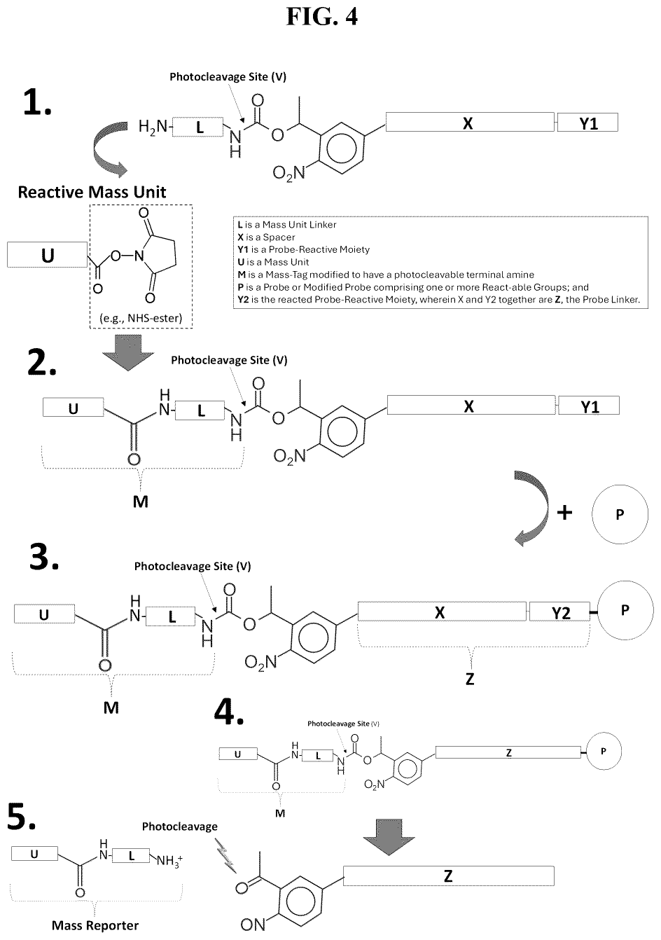

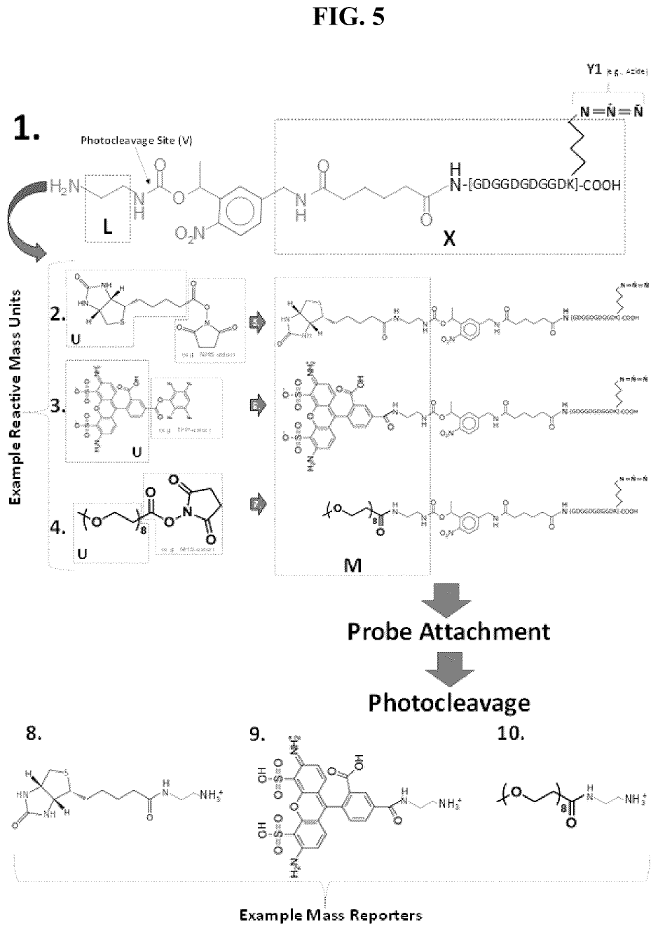

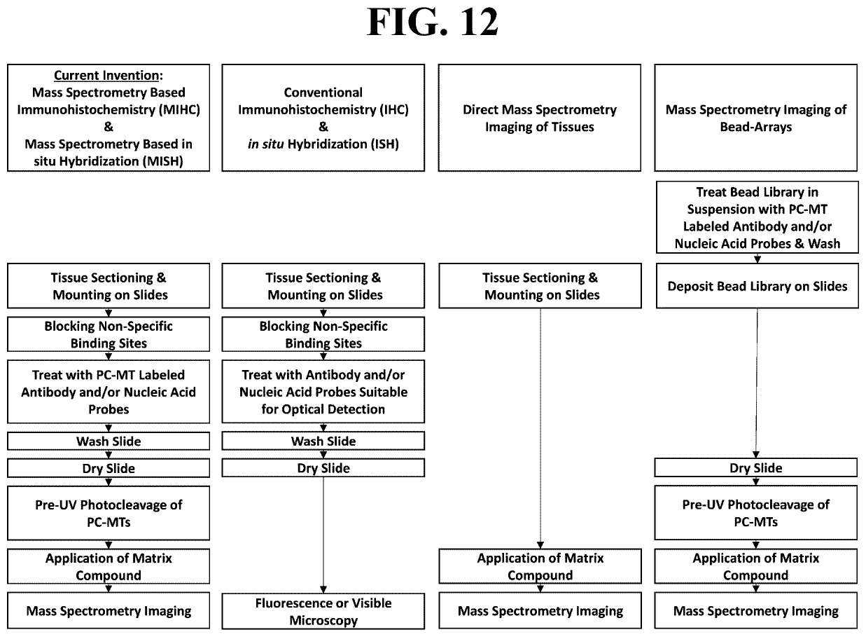

The final step in the process is the utilization of the PC-MT-Probes to treat (“stain”) cells, tissues, or other samples (i.e., PC-MT-Probes are bound to targets in the cells/tissues) followed by MSI to image the photocleaved Mass Reporters (see FIG. 3, Structure 4 for a Mass Reporter photocleaved from a PC-MT Labeled Antibody Probe). Two example embodiments, termed in the Present Invention as mass spectrometry-based immunohistochemistry (MALDI-IHC) and mass spectrometry based in situ hybridization (MALDI-ISH), where antibody and nucleic acid Probes are used, respectively, are analogous to traditional immunohistochemistry (IHC) and in situ hybridization (ISH). In essence, MALDI-IHC and MALDI-ISH differ in the use of PC-MT labeled Probes instead of Probes labeled with fluorophores or chromogenic agents, and the use of MSI instead of optical imaging (e.g., microscopy). The MALDI-IHC and MALDI-ISH process, which is exemplified in detail later in the Experimental Examples, typically involves the basic steps described in the following paragraphs (although as is the case with standard IHC and ISH, many protocol variations are possible as will be recognized by those skilled in the art). See also FIG. 12 for comparison of essential common elements of the protocol for the Present Invention (MALDI-IHC and MALDI-ISH) to that of conventional IHC (e.g., [8]) and conventional ISH (e.g., [17]), as well as to conventional direct MSI (e.g., [27]) and to MSI of bead-arrays (e.g., U.S. Pat. No. 9,523,680 which is hereby incorporated by reference). Note that many protocol variations are possible for example depending on whether FF or FFPE tissues are used, whether it is IHC/MALDI-IHC or ISH/MALDI-ISH based protocol, and/or what types of optical detection methods are used for conventional IHC or ISH (such as directly labeled primary antibodies or secondary detection methods; and colorimetric versus fluorescence readout), therefore FIG. 12 shows only common essential elements of the protocols:

Basic Steps for MALDI-IHC: i) Mounting thin FFPE or fresh frozen tissue sections (e.g., 5-10 μm thick by microtome or cryostat from FFPE or fresh frozen tissue blocks) onto conductive slides (e.g., metal-coated glass slides); note that although indium tin oxide (ITO) coated glass slides as the conductive surface are almost universally used in MSI (e.g., see [64, 65]), in the Present Invention it was found that given the extensive processing steps of MALDI-IHC and MALDI-ISH described below, gold-coated glass slides are beneficial to avoid tissue lifting off the slide during processing and/or tissue damage, while still providing the necessary conductive surface for MSI (e.g., glass slides with a 10 nm gold layer and 2 nm titanium adhesion underlayer as from Platypus Technologies LLC, Madison, WI, or a 50 nm gold layer and 5 nm chromium adhesion underlayer as from Substrata Thin Film Solutions/Angstrom Engineering Inc., ON, Canada); tissue mounting is followed by ii) deparaffinization (e.g., with xylene) in the case of FFPE; iii) rehydration (if deparaffinization was performed) typically with a series of ethanol/water mixtures and aqueous saline buffers; iv) fixation in formalin or paraformaldehyde in the case of fresh frozen tissues; v) antigen retrieval to reverse some of the detrimental effects of formalin/paraformaldehyde fixation (e.g., heating in citrate buffer, pH 6, or the use of formic acid); vi) treatment with blocking buffer to reduce background (typically saline buffer with non-ionic detergent such as Tween-20 or Octyl β-D-Glucopyranoside (OBG) as well as protein blockers such as bovine serum albumin [BSA] and animal serum); vii) staining simultaneously with a mixture of different PC-MT Labeled Antibody Probes (PC-MT-antibodies) for multiplexing (typically diluted in blocking buffer); viii) washing in saline buffer with non-ionic detergent such as Tween-20 to remove any unbound PC-MT-antibodies followed by washing in volatile aqueous buffers such as ammonium bicarbonate to remove non-volatile salts which can interfere with some forms of mass spectrometry; and ix) drying of the tissue slides prior to MSI.

Steps for MALDI-ISH: i) Tissue mounting, ii) deparaffinization, iii) rehydration and iv) formalin/paraformaldehyde fixation are performed as described for MALDI-IHC; this is typically followed by v) partial protein digestion with Proteinase K; vi) fixation of nucleic acids with EDC (1-Ethyl-3-(3-dimethylaminopropyl)carbodiimide); vii) tissue acetylation to cap free amines and reduce background (caused by non-specific binding of Probes); viii) treatment with blocking buffer typically containing at least irrelevant nucleic acids (e.g., yeast tRNA and/or salmon sperm DNA) to reduce background; ix) staining (hybridization) simultaneously with a mixture of different PC-MT Labeled Oligo Probes (PC-MT-oligos) for multiplexing (typically diluted in blocking buffer or similar); x) washing in saline buffer to remove any unbound PC-MT-oligos followed by washing in volatile aqueous buffers such as ammonium bicarbonate to remove non-volatile salts which can interfere with some forms of mass spectrometry; and xi) drying of the tissue slides prior to MSI.

However, as discussed earlier, Probes of the Present Invention need not be restricted to antibodies (used for MALDI-IHC) and nucleic acids (used for MALDI-ISH), and can for example be lectins, receptors, or ligands. Therefore, the Present Invention is not restricted to MALDI-IHC and MALDI-ISH methodologies, but other embodiments where tissues are treated (“stained”) with PC-MT conjugated lectins, receptors, or ligands for example, or any Probe type or combination thereof.

### Mass Spectrometry Imaging (MSI)

Following the procedures as outlined above, MSI is performed, in one embodiment using MALDI-MSI:

For MALDI-MSI, matrix compound is applied to the tissue typically in a thin and uniform layer. Example matrix compounds include alpha-cyano-4-hydroxycinnamic acid (CHCA), 2,5-Dihydroxybenzoic acid (DHB), 1,5-Diaminonaphthalene (DAN) or 3,5-Dimethoxy-4-hydroxycinnamic acid (sinapinic acid). In a preferred embodiment, matrix sublimation followed by recrystallization [37, 66] is used to achieve both excellent spatial resolution (i.e., provided by the sublimation which limits analyte delocalization during matrix application) and high sensitivity (i.e., provided by the recrystallization which allows the Mass Reporters to sufficiently co-crystallize with matrix, but without significant analyte delocalization). Other methods of matrix application may be used, such as by using a commercially available sprayer (e.g., HTX TM-Sprayer, HTX Technologies, LLC, Chapel Hill, NC).

A variety of mass spectrometry technologies and instrumentation may be used for the MSI step, not just MALDI-MSI. Other MSI methods include, but are not limited to, desorption electrospray ionization mass spectrometry imaging (DESI-MSI) [29], laser ablation electrospray ionization mass spectrometry imaging (LAESI-MSI) [67], and atmospheric pressure (AP) matrix-assisted laser desorption/ionization (MALDI) mass spectrometry imaging (AP-MALDI-MSI) [31].

Regardless of the type of MSI technology and instrumentation, PC-MTs are photocleaved to liberate the Mass Reporter from the PC-MT-Probes for MSI analysis (e.g., see FIG. 3, Structure 4). In a preferred embodiment, PC-MTs may be pre-photocleaved by UV treatment prior to MSI, preferably also before matrix application in the case of MALDI-MSI, to avoid matrix absorption of the incident UV. Photocleavage is also preferably performed on dried slides to prevent delocalization of the photocleaved Mass Reporters by diffusion. Pre-photocleavage can be achieved for example by using a model XX-15 self-filtering 365 nm peak lamp (UVP/Analytik Jena US LLC, Upland, CA), but many other light sources are possible. In a preferred embodiment, pre-photocleavage is achieved in a short time (e.g., 5 min) with relatively low intensity UV light (e.g., 3-10 mW/cm²). PC-MTs may alternatively be photocleaved in-line with the MALDI-MSI analysis by the instrument's laser beam (or by the laser beam of any other laser-based mass spectrometers, such as in LAESI-MSI). This alternative approach may be expected to improve spatial resolution of the MSI, since the Mass Reporters are not photocleaved prior to matrix application in the case of MALDI-MSI (whereby matrix application could otherwise cause diffusion of pre-photocleaved Mass Reporters). However, results in U.S. Pat. No. 11,906,527 hereby incorporated by reference indicate that this approach of in-line photocleavage provides poor sensitivity in the case of MALDI-MSI. Superiority of pre-photocleavage, before matrix application and before MALDI-MSI, may be explained as follows: i) pre-photocleavage avoids the light blocking effects of the matrix compound (it is in fact the function of the matrix to absorb the impinging UV laser light during MALDI-MSI, converting it to heat such that the Mass Reporters are vaporized and ionized for analysis); and ii) having the Mass Reporter pre-photocleaved will allow the freed Mass Reporter to co-crystallize with the subsequently-applied matrix (those skilled in the art will recognize that co-crystallization of analytes with matrix is essential for efficient desorption/ionization of the analyte in MALDI-MS), conversely, photocleavage in-line with the MALDI-MSI analysis by the instrument's laser beam necessarily occurs after matrix application/crystallization, likely not allowing for efficient co-crystallization of the matrix and the Mass Reporter (since at the time of matrix application/crystallization, the Mass Reporter is still attached to the Probe which may be still attached to the tissue). However, other laser-based MSI methods and instrumentation, for example those that do not require matrix compound, may be expected to provide efficient in-line photocleavage and sufficient sensitivity, presuming the laser wavelength is sufficiently matched to the photocleavage wavelength.

Note that with the preferred PC-Linker (e.g., FIG. 3, Structure 1, central components), photocleavage leaves a small residual portion of the PC-Linker attached to the Mass Reporter thereby generating a free primary amine on the Mass Reporter upon photocleavage (see FIG. 3, Structure 4), which may assist in Mass Reporter ionization in positive mode MALDI-MSI and therefore this design may increase sensitivity.

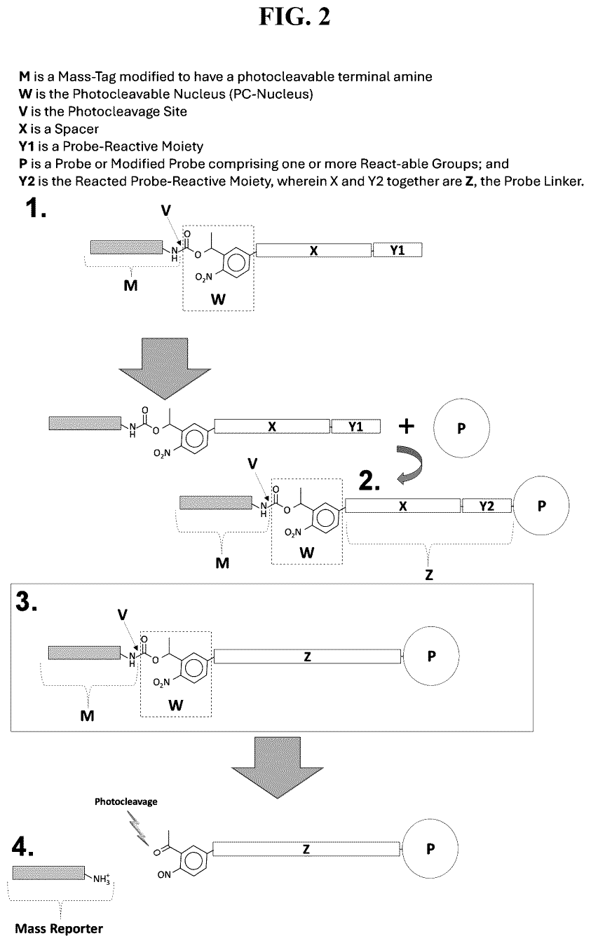

Note that in FIG. 2 for example, the Mass-Tag of the PC-MT is linked (prior to photocleavage) to the phenyl ring of the photocleavable nucleus (PC-Nucleus) through the Photocleavage Site, and the Probe ultimately linked to the phenyl ring of the PC-Nucleus at a position different from the Photocleavage Site. This is the preferred embodiment of the Present Invention since importantly, the photocleaved 1-(2-nitrophenyl)-ethyl moiety of the PC-Nucleus does not remain attached to the Mass Reporter upon photocleavage as shown in FIG. 2 Structure 4 (instead remains attached to the Probe). Conversely, it is possible to entirely reverse this orientation as taught by Olejnik et al. [68] and by Levy and Caprioli (U.S. Pat. No. 7,569,392). However, in this configuration the photocleaved 1-(2-nitrophenyl)-ethyl moiety of the PC-Nucleus remains attached to the Mass Reporter (not depicted in FIG. 2), and as a result, highly complicated mass spectra containing multiple peaks pertaining to the Mass Reporter attached to various side-reaction byproducts of the photocleaved 1-(2-nitrophenyl)-ethyl moiety are observed (see U.S. Pat. No. 11,906,527 hereby incorporated by reference). This will reduce sensitivity in MSI (by dividing signal for a single Mass Reporter among many mass spectral peaks, i.e., said byproducts) and will confound discrimination of different Mass Reporters (e.g., by peak overlap).

Finally, overall, as discussed in the preceding paragraphs, the Present Invention is not restricted to the types of Probes nor the types of mass spectrometry used for MSI. Therefore, more generically, the PC-MT Labeled Probe based MSI technique of the Present Invention is sometimes referred to as PC-MT based MSI (of which the aforementioned methods of mass spectrometry based immunohistochemistry [MALDI-IHC] and mass spectrometry based in situ hybridization [MALDI-ISH] are some of the many possible methodological subtypes).

PC-MT-Probes described in the Present Invention are also not limited to mass spectrometric imaging applications but can be used in conjunction with non-imaging mass spectrometric applications (MS). For example, a group of PC-MT Labeled Probes can be applied to a heterogenous biological sample including but not limited to a tissue or excised portion of a tissue, tumor, cells derived from a tumor, liquid biopsy, blood and other bodily fluids, bacterial cell culture, and biofilms in order to identify the components of the heterogeneous sample. For example, identifying pathogens present in a biological sample could be identified by targeting with one or more PC-MT-antibody Probes the antigens characteristic of the pathogen and identifying the photocleaved Mass Reporters using non-imaging mass spectrometry including MALDI-MS, DESI-MS and LC-MS (e.g., ESI-MS). In one example, epitopes specific to particular variants of the SARS-COV-2 virus responsible for causing the COVID-19 pandemic could be identified using PC-MT-antibodies targeted at the particular epitopes. In a second example, specific antigens present on tumor infiltrating leucocytes could be targeted with specific PC-MT-antibody Probes using non-imaging mass spectrometry. While spatial information is lost due to this non-imaging approach, it is well known to those skilled in the art of immunodiagnostics or flow cytometry that the positive binding of particular antibodies to a biological sample such as tissues or can be used to identify the presence of particular components of the sample such as pathogens in the case of infectious disease.

### Labels in Addition to PC-MTs on Probes

It is to be understood that the addition of a second label to a Probe such as an antibody, lectin, or oligo Probe, in addition to the PC-MT, includes but is not limited to fluorescent labels, and can provide the ability to combine MSI-IHC with other imaging modalities besides fluorescence imaging as described below. Such multimodal imaging is highly desirable since it allows researchers and clinicians to obtain several layers of spatial information which can be statistically correlated when the images are merged. It also provides a means to perform imaging at different spatial resolutions using different imaging techniques. Additional labels which can be advantageously used for detection and/or imaging purposes of tissues and cells include but not limited to the following:
i) Chromogens, which absorb light and unlike fluorescent labels require only a standard microscope for imaging including for example brightfield microscopes and other forms of optical imaging including phase contrast and confocal microscopy. Most chromogenic methods involve amplification of the detected Probe such as an antibody by conjugating the Probe to an enzyme such as horseradish peroxidase (HRP), which converts 3,3′-diaminobenzidine (DAB) into a brown product visible using brightfield microscopy, or alkaline phosphatase (AP), which converts 3-amino-9-ethylcarbazole (AEC) into a red product visible using brightfield microscopy. In the case of antibodies conjugated also with PC-MT, the presence of the enzyme attached to the Probe would provide a second means to image the tissue or cells using standard microscopy that can be co-registered with the mass spectrometric image of the PC-MT. Multiplex detection could also be achieved by conjugating different amplification enzymes to different antibodies that convert products into chromogens with different absorption. However, the extent of multiplexing is limited due to overlap of the absorption of the different chromogens available.
ii) Raman-active molecules, used as coding labels including those that are detected using but not limited to resonance, pre-resonance, stimulated, surface enhanced Raman spectroscopy and photothermal [69-74]. Such labels can be detected, and spatial location imaged in a tissue at high resolution depending on the wavelength of the exciting laser using a variety of commercially available Raman microscopes such as the Bruker Senterra II Raman Microscope. Due to the distinct Raman spectrum of individual molecules such as molecules containing aromatic groups or azide groups which can produce bands in particular regions of the Raman spectrum, each molecule's Raman spectrum can serve as a unique barcode. Furthermore, addition of stable isotopes to the compound will result in specific bands in the Raman spectrum due to changes in the mass producing a variety of different codes depending on the stable isotopes incorporated [75, 76]. Particularly useful are C-D groups which shift the normal C-H group from the 3000 cm-1 region to the 2200 cm⁻¹ region which is much less populated by vibrational bands contributed from other chemical groups. Unlike fluorescence spectra, Raman spectra reflect all of the Raman active vibrations of the molecule and can therefore serve as a distinct molecular fingerprint. In some cases, this signal can be enhanced due to the resonance effect which involves the use of exciting wavelengths that fall inside the envelop of absorption spectrum of the molecule. In other cases, SERS amplification such as using nanoparticles where the molecules are attached (or embedded) can be employed (see for example [77]). An additional example involves the use of electronic pre-resonance stimulated Raman scattering imaging (EPR-hSRS). In one example, EPR-hSRS was used to image membrane voltage-changes using near-infrared absorbing microbial rhodopsins expressed in E. coli [78]. A similar approach could be used to image antibodies conjugated with different near-IR absorbing chromophores to code different antibodies that also contained PC-MTs. In this regard, utilization of PC-MT containing stable isotope labels to shift some of the vibrational modes of the PC-MT into relatively uncongested regions of the spectrum to avoid overlap with other vibrational bands is highly desirable.
iii) Monoisotopic metal reporters or tags such as used for multiple ion beam imaging (MIBI), a form of secondary-ion mass spectrometry (SIMS), or Imaging Mass Cytometry (IMC) which uses inductively coupled plasma mass spectrometry (ICP-MS) ([79, 80]; see [81] for a review of various multiplex antibody imaging approaches). IMC, which has been commercialized by Standard BioTools, is limited to around 40 biomarkers due to the utilization of rare earth metal tagged antibodies but has the ability to achieve subcellular resolution similar to fluorescence IHC or immunocytochemistry (ICC) [24]. Since IMC uses inductively coupled plasma mass spectrometry (ICP-MS) which destroys the tissue, it does not allow post-IMC measurements and it does not permit post-imaging of the tissue or cells. Most critically, while providing very high cellular resolution (1 μm), it is extremely slow and applicable mainly to regions of interest (ROIs) less than 1 mm². This in turn limits throughput and thus the number of cells that can be analyzed in a typical tissue specimen, for example a clinical biopsy. Thus, dual-labeled Probes such as antibodies that contain both PC-MTs and metal-tags would be highly advantageous since they would enable an initial image run using MSI and a secondary imaging run using IMC.
iv) Oligos, with specific coding sequences such as DNA conjugated directly to an antibody or other protein Probes such as a lectin. A variety of methods have been described to conjugate DNA to proteins and a variety of commercial kits are available for this purpose. For example, AlphaThera offers the oYo-Link kit to conjugate antibodies with custom oligos for a variety of purposes including multiplexing. Both ssDNA and dsDNA oligo can be used up to 80 bases or bp. A second example are Abcam oligo conjugation kits (ab218260). These kits enable the easy and efficient generation of antibody-oligo conjugates in less than 2 hours (from ABCAM website https://www.abcam.com/oligonucleotide-conjugation-kit-ab218260.html). The kit can be used to generate conjugates of different ratios of antibody:oligo and would be compatible with antibodies pre-conjugated with PC-MTs. The kit is compatible with both single-stranded oligos that are 10-120 bases long or double-stranded oligos that are 80 bp long. One requirement is that the oligos possess a terminal 3′ or 5′ modification which acts as an attachment site to the Probe such as but not limited to an amino (—NH), sulfhydryl (—SH), or azide (—N3) group which is compatible with the particular attachment chemistry. Such reactive groups can be added synthetically using specialty phosphoramidite reagents such as offered by Glenn Research or can be incorporated into commercially purchased oligos by a variety of variety of oligo synthesis companies such as Integrated DNA Technologies.

The use of dual-labeled PC-MT/metal-tag antibodies described herein and the workflows enabled by these dual-labeled antibodies have many advantages over conventional methods of imaging of tissues. Since MSI is a technology that provides cellular resolution capability (e.g., ≥5 μm) and IMC and MIBI provide subcellular resolution (˜1 μm), the ability to perform multimodal imaging with both techniques (e.g., MSI and IMC) on the same tissue sample offers many advantages. For example, performing high-plex imaging using MALDI-IHC methods described herein can be used as a guide for locating regions of interest (ROIs) to perform the MIBI or IMC where imaging takes much longer. For example, the MSI scan and imaging of the PC-MTs can be performed at lower resolution (e.g., 40 μm) over a significant area of the tissue (e.g., 1 cm²) in less than one hour providing information at high levels of multiplexity, whereas the MIBI and IMC scans which must be performed at much higher resolution (e.g., 1 μm) are much slower and capable of only scanning a limited region at high multiplexity. In one example using a dual-labeled antibody with both PC-MTs and metal tags, the tissue will be scanned first over 1 cm² with a highly multiplex Probe panel (˜40) of dual-labeled antibodies at 40 μm resolution in less than one hour. After analysis of the data, 4 ROIs each 1 mm² will be chosen on basis of the MSI scan to be scanned using MIBI or IMC at ˜0.2 μm resolution in less than 4 hours. Together this imaging data when merged will provide an efficient means of analyzing at very high multiplexity the detailed position and association of target proteins at both low and high resolution in a tissue for use in basic biological research and/or clinical applications. In comparison, scanning the entire tissue section with IMC or MIBI alone would require at least one day of scanning time.

In a second preferred example, dual labeling of antibodies will be performed with both PC-MTs and oligos and both MSI and a method known as co-detection by indexing (CODEX) will be performed in successive steps. CODEX can be performed routinely in a laboratory using well-developed methods in conjunction with ordinary fluorescence microscopes [25] and antibodies conjugated with oligos are also available commercially from Akoya Biosciences to be used with their specialized instruments such as their PhenoCycler. This method alone is used to obtain highly multiplex proteomic imaging of tissues and cells at high resolution but suffers from the need to perform cycles of Probe application and removal. Specific oligo sequences serve as barcodes for specific antibodies which can then be located in the image by hybridizing a complementary oligo sequence that has an incorporated specific fluorophore that is imaged using a fluorescence microscope or scanner. Normally, sets of 3 or 4 oligos are used in the hybridization step, each oligo containing a distinct dye that is chosen so that each dye can be detected using immunofluorescence methods. Similar to ordinary immunofluorescence techniques, each cycle is limited to only a few dyes due to the overlap of excitation and emission spectrum of each dye and difficulty of preventing spectral overlap that prevents spectral separation of the individual type of antibodies bound to the tissue. In order to avoid this problem, CODEX requires that the set of oligos in each cycle is removed and a new cycle of hybridization and fluorescence imaging is performed, providing a set of images from the same tissue which can be merged to produce a highly multiplexed image of antibody targeted biomarkers in the tissue [25].

In a preferred embodiment of the Present Invention that involves the use of dual-labeled PC-MT antibody Probes, a panel of antibodies containing at least some dual-labeled antibodies with a combination of PC-MT and oligos residing on each antibody, is produced and used to stain the tissue with procedures similar to conventional IHC. In this case, each dual-labeled antibody contains coding agents that code for the specific antibody both due to the presence of a unique PC-MT and a corresponding unique oligo sequence. There are virtually an unlimited number of nucleotide sequences that can be used to code for antibodies, although most sequences are designed so the complementary hybridization Probe does not hybridize with endogenous nucleic acid sequences in the sample. During or after IHC, a step may be included that fixes the antibody to the tissue using a post-staining antibody fixation procedure to prevent washing out of the antibody in subsequent steps (see below). After photocleavage of the PC-MT and application of the matrix, the tissue section is imaged using MSI. The matrix is then removed and then the tissue imaged again using methods described for CODEX [25]. This multiplex CODEX is facilitated by the presence of the oligo coding of each antibody that was dual-labeled. Note that in this case the PC-MT has been partially or fully removed after photocleavage but the oligo coding on each antibody remains.

The use of dual-labeled PC-MT/oligo tag antibodies described herein, and the workflows enabled by these dual-labeled antibodies have many advantages over conventional methods of imaging of tissues using methods such as CODEX or other multiplexed oligo-antibody complexes alone. Since MSI is a technology that currently provides spatial resolution down to −5 μm and CODEX and other cyclic fluorescence oligo imaging methods based on optical microscopy can provide subcellular resolution (as low as 0.2 μm), this allows a very high-plex rapid scan of a tissue or cells by MSI followed by a higher resolution scan of selected targets using for example CODEX at cellular or subcellular spatial resolution over a specific region, thus drastically reducing the number of iterative cycles required for CODEX.

In addition to CODEX, there are a variety of other cyclic immunofluorescence (CyCIF) methods which will benefit in a reduction in the number of cycles required due to the use of dual-labeled Probes as described in the Present Invention. They all share the common feature that the method involves adding and removing a small set of fluorophores that can be imaged at each cycle of fluorescence imaging. For example, this removal can include de-hybridization of the hybridization Probes (CODEX), by bleaching the fluorophore using light, or chemical removal or photocleavage of the fluorophore. In this way, multiple cycles of antibodies can be used to stain the tissue and imaged to obtain high levels of multiplexing.

While these CyCIF methods can result in very high-resolution imaging of many Probes, the methods suffer from several disadvantages. For example to achieve high multiplexity, CyCIF require extensive cycling procedures which involve iterative staining, imaging, and Probe or dye removal/inactivation [16, 21-23]. This includes Akoya's OPAL multispectral platform, t-CyCIF [82], and CODEX [83]. For example, t-CyCIF requires 20 iterative cycles of staining, imaging and dye bleaching to achieve 60-plex tissue imaging [82]. As stated by the developers of this method [82], “One potential concern about cyclic immunofluorescence is that the process is relatively slow; each cycle takes 6-8 hr and we typically perform one cycle per day”. Therefore, 60-plex imaging would require 20 days. Not only are such methods complex, slow and laborious, but cycling can damage the tissues and incomplete cycling can confound the results [16, 24]. In addition, these methods are not easily amenable to multiomic imaging, since they don't enable, on the same imager, detection of multiple types of molecular species such as proteins and metabolites from the same tissue specimen. Other techniques such as Nanostring's GeoMx® can also reach high multiplexity by profiling oligo Probes that are photocleaved from specific regions of a tissue (greater than 50×50 μm²). However, this technique is extremely slow (typically 10 slides/day), extremely expensive per slide, and requires specialized equipment.

### Post-Fixation of Probes

In one preferred embodiment of the Present Invention that involves the use of dual-labeled PC-MT Probes, a panel of antibodies containing at least some dual-labeled antibodies with a combination of PC-MT and a second label on each antibody such as conjugated oligo or metal tags is produced and used to label the tissue with procedures similar to conventional IHC. During or after IHC, a step may be included that fixes the antibody to the tissue using a post-staining antibody fixation procedure to prevent washing out of the antibody in subsequent steps. Such a procedure is well described in the literature [25]. One procedure described in [25] involves the use of paraformaldehyde (PFA) solutions as a fixative. These post-staining antibody fixation steps are included since subsequent steps in the application of dual-labeled Probes may disrupt the interaction of the antibody with the target antigen, thereby removing or displacing the position of the antibody unless this post-staining antibody fixation step is performed. Such procedures which might disrupt the antibody-antigen interaction include washing of the tissue sample, application of matrix, and removal of the matrix, for example by washing. Such a post-staining antibody fixative is routinely included in CyCIF methods such as CODEX to prevent antibody removal during iterative hybridization labeling steps and subsequent washing for each cycle [84]. After photocleavage of the PC-MT and application of the matrix, the tissue section is imaged using MSI. The matrix is then removed and then the tissue imaged again using for example immunofluorescence imaging (IF). This second imaging step is facilitated by the presence of the second label on each antibody that was dual-labeled. Note that in this case the PC-MT has been partially or fully removed after photocleavage but the second label, such as the oligo or metal tag coding on each antibody remains, or partially remains. The reverse procedure is also possible in the case of MIBI whereby local regions of the tissue are measured without disrupting or cleaving the PC-MTs, and then the entire tissue or portions of the tissue scanned by MALDI-IHC. However, this is not possible using IMC methods which will result in destruction or degradation of the tissue including the dual-labeled Probes due to laser ablation.

### Multiomic Tissue Imaging Using PC-MT-Probes

A key advantage of the Present Invention is the ability to perform multiomic imaging of tissue specimens, whereby “omics” is the measurement of some characteristic of a large family of cellular molecules, such molecules including, but not limited to, genes (genomics), proteins (proteomics), small metabolites (metabolomics), glycans (glycomics) or RNA (transcriptomics) [85]. In the case of tissue imaging, this measured “characteristic” for example can be the spatial mapping, morphological analysis, and co-localization analysis of these cellular molecules, and may also include quantification (e.g., quantifying biomarker levels or scoring the number of biomarker-positive cells). Multiomics is therefore the combined measurement and analysis of different omic groups (e.g., combined proteomics and glycomics). In the Present Invention in the context of tissue imaging, multiomics can be achieved by using for example, a) different PC-MT Labeled Probe types or classes (e.g., antibodies and lectins) and/or b) different MSI “modes” (e.g., untargeted direct MSI of endogenous tissue biomolecules as well as MSI of biomolecules targeted by specific PC-MT Labeled Probes). In a preferred embodiment, the different omic measurements are made on the same tissue section. However, it is also possible to make these measurements on separate, preferably sequential/adjacent tissue sections sliced from the same tissue specimen/block. Moreover, the different omic measurements may be performed simultaneously on the tissue section/specimen (e.g., by treating the tissue with a mixture of different PC-MT Labeled Probe classes, such as antibodies and lectins) or in sequence (one omic measurement followed by another). For example, with fluorescence imaging, this has been done with various methodological permutations using lectins and antibodies [86]. In the case where the different omic measurements are performed in sequence on the tissue specimen, the Present Invention is not intended to be limited to the order in which these measurements are performed, since many permutations could produce useful results as will be apparent in the following paragraphs.

Multiomics in the Present Invention is facilitated by the use of MSI methods that employ “soft” ionization for molecular analysis, since these methods generally cause no or limited molecular fragmentation and more specifically, do not atomize molecules for detection (allowing for example the detection of intact peptide or polymeric based mass-tags as well as intact endogenous biomolecules in the tissues such as lipids, drugs, or metabolites). Soft ionization methods include but are not limited to laser desorption ionization (LDI), matrix-assisted laser desorption ionization (MALDI), desorption/ionization on silicon (DIOS), fast atom/ion bombardment (FAB) and electrospray ionization (ESI) [87] including derivatives of ESI as discussed earlier such as DESI and LAESI. The aforementioned list is not intended to limit the Present Invention to any particular type of “soft” ionization mass spectrometry. For the purposes of the Present Invention, “soft” ionization-based methods are defined as those which ionize and detect molecular ions, that is, electrically charged molecules comprised of 2 or more atoms held together by chemical bonds.

Double Sequential MSI: In a simple multiomic embodiment, it is useful to first perform label-free direct MSI of endogenous small biomolecules (e.g., metabolomics), followed by MSI of targeted biomolecules using PC-MT based MSI (e.g., protein targets such as in proteomics), preferably on the same tissue section. The targeted PC-MT based MSI is necessary to detect biomolecules that are generally not accessible to direct MSI, such as macromolecular targets which themselves do not ionize well, are not detected well, may fragment in undesirable fashion even with soft ionization, and/or are not resolved well in the mass spectrometer, for example. Including PC-MT based MSI in the multiomic workflow is also important where targeting at least some known biomarkers is desired or necessary. Overall, this embodiment of multiomic tissue imaging may be important for example to co-localize drug compounds (small molecule detection by direct label-free MSI) and drug targets (macromolecule detection by PC-MT based MSI, since drug targets are typically proteins). In a preferred embodiment, label-free direct MALDI-MSI is performed first, and uses fresh frozen (FF) tissue sections so that the tissues have not yet undergone tissue fixation (more ideal for direct label-free MSI of endogenous molecules; although the use of formalin-fixed paraffin embedded [FFPE] is possible [88]). In a preferred embodiment, tissue sections are then washed (e.g., in organic solvent) for removal of any remaining matrix compound and then PC-MT based MSI methods are employed such as MALDI-IHC and/or MALDI-ISH, which includes another cycle of MALDI-MSI, on the same tissue section.

In another preferred multiomic embodiment, the targeted PC-MT based MSI method is combined with untargeted “bottom-up” omics methods.

Bottom-up omics refers to the fact that biomolecules are first broken down (digested) into smaller fragments that are more accessible to mass spectrometry. The identity and structure of the whole biomolecules may be subsequently deduced from the mass spectrometry analysis. In other bottom-up scenarios, the identities of the biomolecules need not be known, since merely a fingerprint of various mass species (measured with high precision) may be correlated with a disease state or stage for example. In the context of bottom-up tissue MSI, the tissue is treated with a digesting agent to liberate biomolecular fragments which are more accessible to mass spectrometry. Typically, the digesting agents are sprayed onto the tissue in a thin film so as to facilitate digestion without analyte delocalization, so-called in situ digestion. However, it is a challenge to achieve a balance between sufficient digestion and minimal delocalization, and likely no perfect balance exists. These digesting agents may be enzymes, such as nucleases (e.g., restriction enzymes), proteases (e.g., chymotrypsin, trypsin, LysC, or AspN), kinases (e.g., PKC), phosphatases (e.g., alkaline phosphatase), or glycosidases (e.g., Peptide N-Glycosidase F [PNGase F]) or they may be chemical agents such as cyanogen bromide (CNBr) or hydroxylamine [89, 90]. In situ tissue digestion followed by MSI has been previously reported. For example, Drake et al. [91], sprayed the glycosidase PNGase F onto FF and FFPE tissues to enzymatically cleave N-linked glycans from proteins in the tissues, followed by MALDI-MSI of the liberated glycans. Other examples, in this case using proteases as the digesting agent, include the use of collagenase to digest a specific protein type or trypsin to achieve general protein digestion [92, 93].

When bottom-up MSI is combined with PC-MT based MSI for a multiomic tissue imaging approach, the Present Invention is not intended to be limited to a particular order of operations. For example, the PC-MT based MSI may be performed before the bottom-up MSI, or after (both options requiring 2 rounds of MSI). Conversely, the tissue may be stained with PC-MT Labeled Probes and then in situ digestion performed followed by only a single round of MSI. In cases where PC-MT based MSI is performed first, it may be desirable to subsequently remove the PC-MT Labeled Probes in the case where the Probes may interfere with the subsequent bottom-up MSI. To do so, Probes may be first detached from the tissues by a denaturing treatment and the Probes then washed away. Such denaturing treatments can include, but are not limited to, chaotropic agents, solutions of pH≤5, solutions of pH≥10, reducing agents, oxidizing agents, heat, organic solvents, and/or detergents (e.g., ionic detergents such as SDS, non-ionic detergents such as Triton X-100, or zwitterionic detergents such as CHAPS).

### EXPERIMENTAL

### Materials for Experimental Examples

Water (LCMS grade) and xylene (semiconductor grade) were from Acros Organics (Pittsburgh, PA). Methanol (LCMS grade) was from J. T. Baker (Avantor, Radnor, PA). Ethanol (bioreagent for molecular biology), acetone (HPLC grade), N,N dimethyl formamide (anhydrous, ≥99.8%), 1,5-diaminonaphthalene (DAN, 97%), 2,5-dihydroxybenzoic acid (DHB, 98.0%), isopropyl alcohol (bioreagent for molecular biology), sodium chloride (BioXtra, ≥99.5%), sodium bicarbonate (99.7%-100.3%, molecular biology tested), ammonium bicarbonate (BioUltra, ≥99.5%), bovine serum albumin (heat shock fraction, protease free, fatty acid free, essentially globulin free, pH 7, ≥98%), phosphate buffered saline (PBS) (BioPerformance Certified, pH 7.4, P5368), glycine (ultra for molecular biology, ≥99%, Fluka Biochemika), paraformaldehyde (powder, 95%), Citrate Retrieval Buffer (pH 6.0, 10× Antigen Retriever), Octyl β-D-Glucopyranoside (OBG) (50% [w/v] stock solution), 0.5 mL Ultrafree-MC Centrifugal 0.45 μm Filter Devices, Amicon Ultra-0.5 Centrifugal Filter Units with Ultracel-50 Regenerated Cellulose Membrane, Poly-L-lysine (MW 150,000 to 300,000) 0.1% (w/v), IGEPAL CO-630, α-cyano-4-hydroxycinnamic acid (CHCA), ammonium phosphate, ProteoMass™ Angiotensin II MALDI-MS Standard, paraformaldehyde (PFA) powder 95%, and acetic acid 99.99% were from MilliporeSigma (Burlington, MA). Tris HCl and Tris base (molecular biology grade), 5N sodium chloride (molecular biology grade), 0.5 M EDTA (pH 8; molecular biology grade), Tween-20 (molecular biology grade), SSC buffer 20× (molecular biology grade), and molecular biology grade nuclease-free water were from Promega (Madison, WI). Gold coated microscope slides (Au-500 Å) were from Angstrom Engineering Inc. (Kitchener, ON, Canada). IntelliSlides were from Bruker Daltonics (Billerica, MA). FFPE human tonsil tissue blocks were from amsbio LLC (Cambridge, MA) and FFPE human breast cancer tissue blocks were from OriGene (Rockville, MD). Transgenic hAbeta^{SAA} (APP-SAA) FFPE and Fresh Frozen (FF) (embedded in 2% w/v CMC) mouse brain blocks were from The Jackson Laboratory (Bar Harbor, ME). C57 FFPE and FF (embedded in 2% w/v CMC) mouse brain blocks were from Zyagen (San Diego, CA). FFPE and FF tissue blocks were microtome sectioned (5 μm thickness) or cryosectioned (10 μm thickness) and mounted on slides at Zyagen (San Diego, CA). Antibodies were from BioTechne including R&D Systems and Novus Biologicals (Minneapolis, MN), abcam (Cambridge, MA), and Cell Signaling Technology (CST) (Danvers, MA). Mouse IgG whole molecule (015-000-003), normal mouse serum (015-222-001), rabbit IgG whole molecule (011-000-003) and normal rabbit serum (011-000-001) were from Jackson ImmunoResearch Laboratories, Inc. (West Grove, PA). NAP-5 Sephadex G-25 Columns and PD SpinTrap G-25 Columns were from GE Healthcare Life Sciences (Pittsburgh, PA). ProPlate® 16-Well Chambers were from Grace Bio-Labs (Bend, OR). 100× Halt™ Protease Inhibitor Cocktail with EDTA, LCMS grade trifluoroacetic acid (TFA), LCMS grade isopropyl alcohol, HPLC grade chloroform, and LCMS grade acetonitrile were from Thermo Fisher Scientific (Waltham, MA). Alkaline Retrieval Buffer (pH 9.0, 100× Tris-EDTA Buffer) was from abcam (Cambridge, MA).

### EXAMPLES

The following examples are provided in order to demonstrate and further illustrate certain preferred embodiments and aspects of the present invention and are not to be construed as limiting the scope thereof.

### Example 1. MALDI-IHC Using PC-MT Labeled Antibody Probes (PC-MT-Antibodies) Created with an NHS-Activated Peptide-Based PC-MT Labeling Reagent

### Synthesis of the NHS-Activated Peptide-Based PC-MT Labeling Reagent

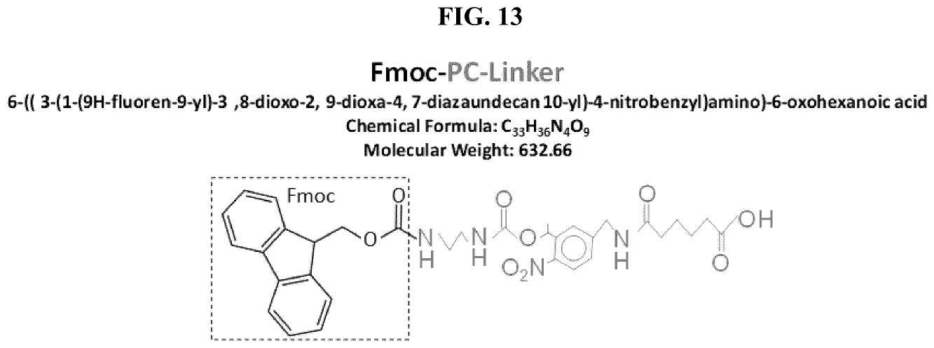

Synthesis of NHS-activated peptide-based PC-MT Labeling Reagents was performed as described as in U.S. Pat. No. 11,906,527 which is hereby incorporated by reference. See FIG. 3 (Structure 1) in the Present Specification for an example PC-MT Labeling Reagent of this type. Note that in FIG. 3 (Structure 1) the bracketed amino acid sequence within the Mass-Tag (M) component was the only variable portion among the different PC-MT Labeling Reagents prepared, whereby the sequence shown is just one example. Different amino acid sequences were used to create different PC-MT Labeling Reagents which were used to encode (label) the different antibody Probes used in this Example (see Table II for sequences and antibodies). Briefly, to synthesize the NHS-activated peptide-based PC-MT Labeling Reagents, Fmoc-based solid-phase peptide synthesis (SPPS) was used, currently the preferred mode of chemical peptide synthesis [94]. An Fmoc-protected photocleavable linker (Fmoc-PC-Linker; FIG. 13) comprising the 1-(2-nitrophenyl)-ethyl based Photocleavable Nucleus (PC-Nucleus) was incorporated in the same manner as the amino acids using the standard SPPS. The structure in FIG. 13 is one preferred embodiment of the Fmoc-PC-Linker to incorporate the 1-(2-nitrophenyl)-ethyl based PC-Nucleus, however, other compositions are possible. A suitable Fmoc-PC-Linker minimally contains an Fmoc-protected primary amine terminal, a free carboxyl terminal and the 1-(2-nitrophenyl)-ethyl based PC-Nucleus in between. Moreover, it is to be understood that other protecting groups aside from the Fmoc, and other methods of peptide synthesis, are possible, such as Boc protecting groups and the associated peptide synthesis chemistries [95].

In this Example, the NHS-ester Probe-Reactive Moiety (Y1 in FIG. 3, Structure 1) was created on the ε-amine of a lysine (K) side chain in the PC-MT Labeling Reagent using DSS (disuccinimidyl suberate), similar to the use of N,N′-Disuccinimidyl Carbonate (DSC) for conversion of amines to NHS-esters [96]. Note the N-terminal α-amine of the peptide-based PC-MT Labeling Reagent was acetylated using standard SPPS methods (“Ac” in FIG. 3, Structure 1) to avoid reaction with the NHS-ester Probe-Reactive Moiety.

In this Example, the peptide-based NHS-activated PC-MT Labeling Reagent is referred to as PC-MT-NHS.

### Covalent Conjugation of PC-MT-NHS to Antibody Probes to Create PC-MT Labeled Antibody Probes (PC-MT-Antibodies)

100 μL of antibody solution (1 μg/μL in PBS) was supplemented with 1/9^{th} volume of 1M sodium bicarbonate followed by sufficient PC-MT-NHS added from a 1 mM stock in anhydrous DMF for a 10-fold molar excess relative to the antibody. The reaction was carried out for 1 hr with gentle shaking. The reaction was then quenched by adding 1/9^{th} volume of 1M glycine followed by gentle shaking for 15 min. Buffer exchange and removal of unreacted PC-MT-NHS and byproducts (e.g., hydrolyzed PC-MT-NHS or that which reacted with the glycine quencher) was achieved using diafiltration in Amicon Ultra-0.5 Centrifugal Filter Units with an Ultracel-50 Regenerated Cellulose Membrane (50 kDa MWCO) according to the manufacturer's instructions, against TBS (50 mM Tris, pH 7.5, 200 mM NaCl) as the diluent. Following the last diafiltration cycle, 100 μL of TBS was added to the concentrated PC-MT-antibody and the PC-MT-antibody solution was recovered (˜125 μL total). PC-MT-antibody concentration was measured by absorbance at 280 nm in a Nanodrop One^{C} microvolume spectrophotometer (Thermo Fisher Scientific, Waltham, MA). The PC-MT-antibody was then mixed with equal volume of 2× Storage Buffer (80% glycerol [v/v] in LCMS grade water with 0.1% [w/v] sodium azide, and 2× concentrated Halt™ Protease Inhibitor Cocktail with EDTA [see Materials]). PC-MT-antibodies were stored at −20° C. and protected from light. An example structure of a PC-MT-antibody (PC-MT Labeled Probe) created with this approach is shown in FIG. 3, Structure 3 (note that while one attached PC-MT is shown in FIG. 3 for simplicity, this procedure generally creates multiple attached PC-MTs, e.g., 3-5, on the antibody by PC-MT-NHS conjugation to available primary amines in the antibody).

### Overview PC-MT-Antibody Staining of Tissues, Optional Post-Fixation, and Mass Spectrometry Imaging (Together Called MALDI-IHC)

The cell/tissue samples (e.g., formalin-fixed paraffin-embedded [FFPE] or fresh frozen [FF] tissues) of the Present Invention are most typically mounted (typically thin-sectioned in the case of tissues) on a microscope slide or similar (conductive or otherwise). “Staining” refers to the processes involved in binding the photocleavable mass-tag (PC-MT) conjugated antibodies (PC-MT-antibodies; also generically referred to as PC-MT Labeled Probes) to their targets within the cell/tissue sample. With the PC-MT coding, multiplexing is possible whereby multiple PC-MT-antibodies against different protein targets, each antibody with a unique PC-MT having a unique Mass Reporter, are mixed together and used to stain the cell/tissue sample simultaneously. Following staining, washing, and drying of the sample, the PC-MTs are typically photocleaved from the bound antibodies (thereby releasing the PC-MT Mass Reporter from the cell/tissue sample), followed by application of the MALDI mass spectrometry (MALDI-MS) matrix compound, and finally MALDI mass spectrometry imaging of the photocleaved PC-MT Mass Reporters. Other forms of mass spectrometry imaging (MSI) are possible, such as ESI-based DESI-MS imaging [29], in which case MALDI-MS matrix compound is not needed.

Covalent fixation (cross-linking) of the PC-MT-antibodies to the cell/tissue sample after staining (but before photocleavage), a method hereafter referred to as “post-fixation”, is beneficial in certain cases (but not always needed). Examples include where the cell/tissue sample is further processed after staining under conditions where, without post-fixation, the PC-MT-antibodies would be undesirably dissociated from the cell/tissue sample. In these cases, such as post-staining stringent washing in organic solvent or other harsh/denaturing agents (e.g., chaotropic agents), or additional “secondary” staining procedures (such as for other imaging modalities), the post-fixation will allow the PC-MT-antibodies to remain attached to the cell/tissue sample throughout the downstream processing. Several fixatives are possible including but not limited to formalin, formaldehyde, paraformaldehyde (PFA), glutaraldehyde, N-ethyl-N′-(3-dimethylaminopropyl)carbodiimide (EDC; or other carbodiimides), or epoxy for example. In this example, PFA is used as a fixative. Notably, the PC-MTs used lack any available primary amines (prior to photocleavage) and thus will not react with or become cross-linked by PFA, thereby still allowing photo-release and mass spectrometry analysis of the PC-MT Mass Reporter.

The overall aforementioned process of PC-MT-antibody staining of tissues, optional post-fixation, and MALDI mass spectrometry imaging is referred to MALDI-IHC (for MALDI mass spectrometry-based immunohistochemistry [IHC]).

Note that in this and all other Examples, any steps >10 min involving photocleavable compounds or derivatives were carried out protected from light (if <10 min, including routine handling of the compounds and samples, ambient room lighting but not direct sunlight was allowed).

### Preparation of Formalin-Fixed Paraffin-Embedded Tissue Slides

To promote subsequent tissue adhesion, 75×25×1 mm Bruker IntelliSlides were first coated with Poly-L-lysine as follows: Poly-L-lysine (0.1% [w/v]) and LCMS water were mixed at a 1:1 ratio. IGEPAL CO-630 detergent was then added to a 0.07% (v/v) final concentration, followed by thorough mixing. A cover glass was used to homogeneously spread 20 μL of the prepared mixture onto the slide and the slide then dried for 15 min at 80° C. in a laboratory oven.

FFPE human tonsil tissue blocks were sectioned to 3-5 μm thickness using a microtome, mounted onto the slides and dried. Before use, tissue slides were pre-heated to 60° C. for 2 hours in a laboratory oven to further promote tissue adhesion.

Staining with PC-MT-Antibodies

Unless otherwise noted, all slide processing steps were performed manually in Coplin staining jars with 70 mL total volume.

For deparaffinization and rehydration, FFPE tonsil tissue slides were treated as follows (each treatment step in separate Coplin staining jars): 3× with xylene for 5 min each; 1× with xylene:ethanol (1:1) for 3 min; and then rehydrate 2× with 100% ethanol for 2 min each, 1× with 95% ethanol for 3 min, 1× with 70% ethanol for 3 min, 1× with 50% ethanol for 3 min and 1× with TBS for 10 min.

Heat-induced antigen retrieval (HIAR) was achieved in 1× Citrate Retrieval Buffer or 1× Alkaline Retrieval Buffer (see Materials) in a capped polypropylene Coplin staining jar placed in a 95° C. water bath for 1 hr (lx Citrate Retrieval Buffer) or 30 min (1× Alkaline Retrieval Buffer) and cooled in the same Coplin staining jar for 30 min at room temperature. Slides were then washed 1×10 min with TBS and blocked for 1 hr with Tissue Blocking Buffer (2% [v/v] normal serum [2% each rabbit and mouse] and 5% (w/v) BSA in TBS-OBG; note TBS-OBG is TBS supplemented with 0.05% [v/v] Octyl β-D-Glucopyranoside [OBG]). Slides were then treated for 1 hr at 37° C. or overnight at 4° C. with 100 μL/cm² of a solution containing 2.5 μg/mL of each PC-MT-antibody diluted in Tissue Blocking Buffer (this incubation was not performed in Coplin staining jars, but instead was performed with the solution directly overlaid onto the tissue sections, with the slides in a humidified chamber to avoid evaporation, and with each tissue section surrounded by hydrophobic barrier pen markings to retain the small volumes of fluid).

### Washing and Optional Post-Fixation

Following PC-MT-antibody incubation, the slides were tilted to allow the PC-MT-antibody solution to run off to waste. The slides were next washed, and in some cases post-fixed, using 35 mL per step in a 10 cm glass petri dish with gentle shaking as follows: In the case where post-fixation was not performed, slides were washed 3×5 min each with TBS followed by 1×10 s and then 3×2 min each with 50 mM ammonium bicarbonate (note, all solutions were in LCMS grade water). In the case where post-fixation was performed, the slides were instead washed 3×5 min each with PBS followed by 30 min fixation in 1% (w/v) PFA in PBS, pH 7.4 (prepared fresh by dissolving 1.0 g of PFA powder in 60 mL of PBS with ˜1.0 mL 1 M NaOH on a heating block at ˜60° C. under constant stirring, then adjusting the pH to 7.4 with 1 M HCl [˜1 mL] using a pH meter; then adjusting the final volume to 100 mL with PBS); in the case of post-fixation the slides were then further washed 3×5 min in PBS and 1×10 s and then 3×2 min each with 50 mM ammonium bicarbonate (note, all solutions were in LCMS grade water).

### Photocleavage

Slides were dried for 1.5 hrs in a vacuum desiccation chamber and illuminated with 365 nm light for 10 min at ˜3 mW/cm² or 5 min at ˜30 mW/cm² (using an AmberGen Lightbox, Billerica, MA or LED Cube 100 IC, Honle UV Technology, Marlboro, MA, respectively).

### Matrix Application

Next, DHB or DAN matrices were applied to the dried slides by sublimation followed by recrystallization in a vapor of 5% isopropyl alcohol (IPA) according to published reports [37, 66]. Alternatively, CHCA matrix was applied using an HTX M3+ Sprayer (HTX Technologies, LLC, Chapel Hill, NC) according to the parameters in Table I, and subsequently subjected to recrystallization per the aforementioned published methods.

TABLE I
CHCA Matrix Spraying Conditions with HTX
M3+ Sprayer. Base solvent is 70% acetonitrile,
0.1% TFA and 10 mM ammonium phosphate

| | | | | Track | |
|---|---|---|---|---|---|
| | Flow Rate | Velocity | # | Spacing | CHCA |
| Temp (° C.) | (mL/min) | (mm/min) | Passes | (mm) | (mg/mL) |
| 60 | 0.1 | 1,350 | 8 | 3 | 10 |

| | Nozzle | | | | Dry |
|---|---|---|---|---|---|
| | Hight | | | Gas Flow | Time |
| Solvents | (mm) | Pattern | PSI | (L/min) | (s) |
| 70% ACN, 0.1% | 40 | CC | 10 | 2 | 10 |
| TFA, | | | | | |
| 10 mM Ammonium | | | | | |
| phosphate | | | | | |

### MALDI Mass Spectrometry Imaging (MALDI-MSI)

MALDI mass spectrometry imaging (MALDI-MSI) was achieved with either a rapifleX or timsTOF fleX MALDI-TOF-MS system (Bruker Daltonics, Billerica, MA) typically in reflector mode, with 10-20 μm spatial resolution, and 200-500 laser shots/pixel. Image and spectral analysis were performed using flexImaging, flexAnalysis, and SCiLS Lab software (Bruker Daltonics, Billerica, MA).

### Results

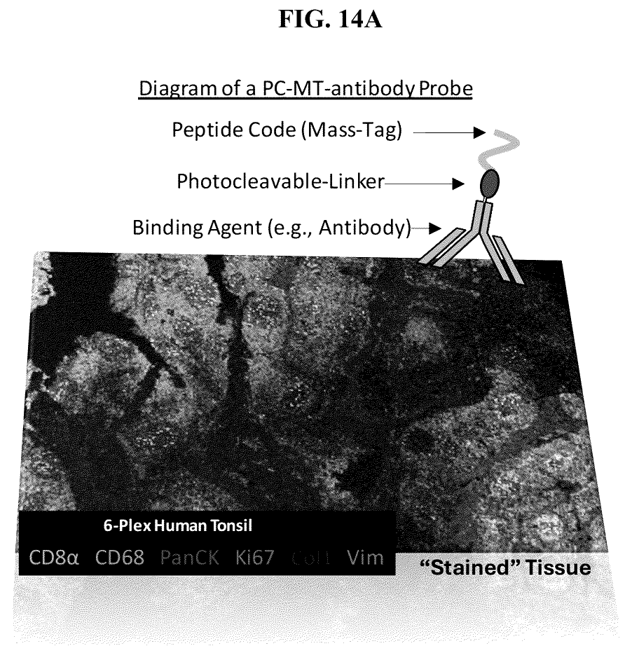

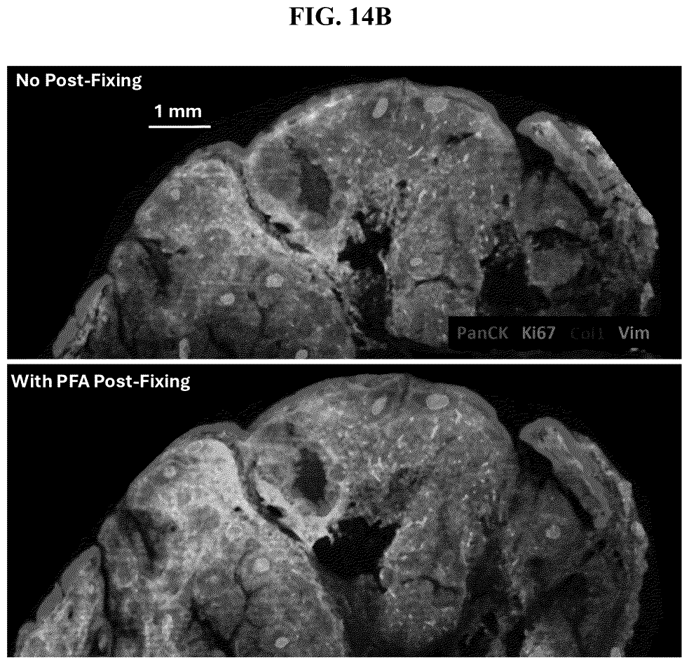

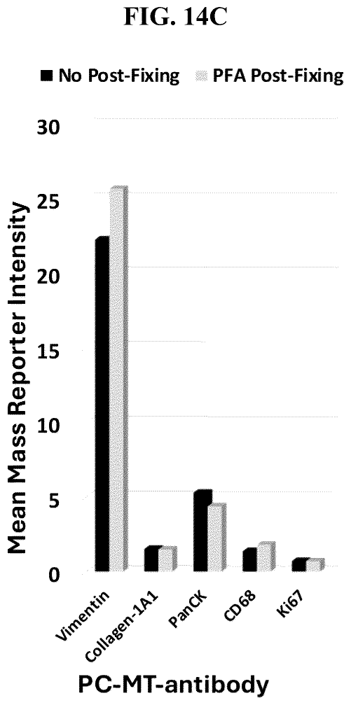

FIG. 14A shows a basic schematic of a PC-MT-antibody Probe bound to a tissue (human tonsil in this case). The tissue was “stained”, without post-fixation in this case, with a 6-plex panel of PC-MT antibodies. The MALDI-MSI image of the tissue in FIG. 14A shows the spatial map of the relative intensities of the PC-MT Mass Reporters (hereafter referred to as MALDI-IHC images) corresponding to the 6 different PC-MT-antibodies (colorized according to the key in FIG. 14A; antibodies were against CD8α, CD68, Pan-Cytokeratin [PanCK], Ki67, Collagen-1A1 [Col1], and Vimentin [Vim]). The antibody patterns match the expected histology for a human tonsil based on the Human Protein Atlas database [97](e.g., Ki67 in germinal centers, PanCK in the squamous epithelial lining of the tonsil and crypts, and vimentin widely distributed since it is expressed in a range of mesenchymal cell types present, such as lymphocytes).

FIG. 14B shows MALDI-IHC images of serial FFPE tonsil tissue sections with and without the post-fixation employed (20 μm scanning resolution) (a 5-plex antibody panel was used in this case, comprising Pan-Cytokeratin [PanCK], Ki67, Collagen-1A1 [Col1], Vimentin [Vim], and CD68 antibodies, with CD68 not shown in FIG. 14B to avoid excessive overlaid colors which can confound visualization). The image patterns of the various antibodies show the expected histology as detailed earlier in this Example and are highly comparable both with and without the post-fixation, indicating no detrimental effects of post-fixation on the MALDI-IHC approach. FIG. 14C shows the mean intensities (from each entire scanned region) for the PC-MT Mass Reporters for each of the 5 antibodies, again showing no significant difference between MALDI-IHC performed with and without post-fixation.

### Example 2. MALDI-IHC Using PC-MT Labeled Antibody Probes (PC-MT-Antibodies) Created with a TFP-Activated Peptide-Based PC-MT Labeling Reagent

### Synthesis of the TFP-Activated Peptide-Based PC-MT Labeling Reagent

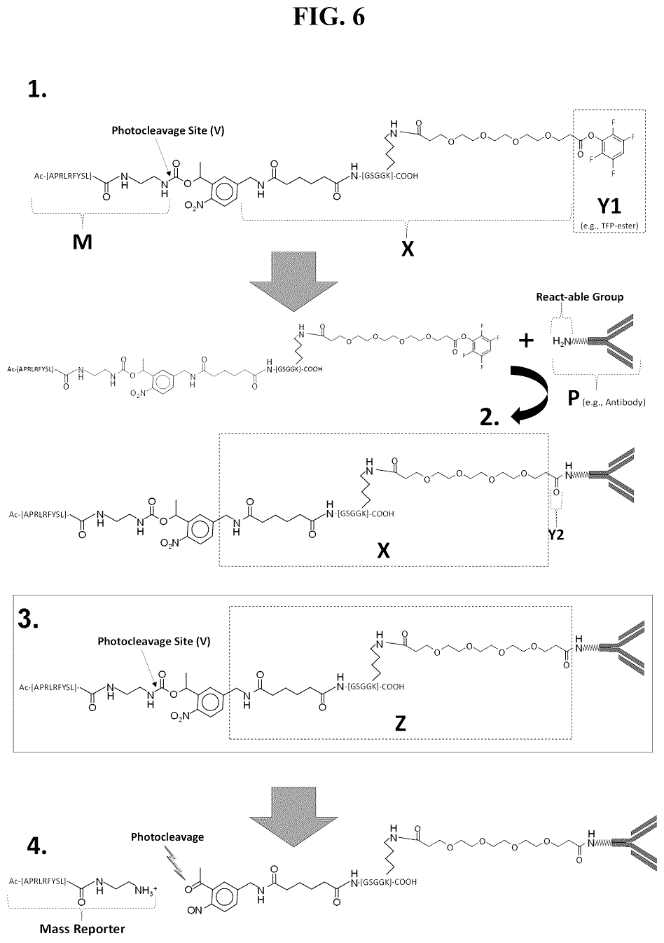

Performed as in Example 1, except that instead of generating an NHS-ester on the ε-amine of the lysine (K) side chain in the PC-MT Labeling Reagent using DSS (disuccinimidyl suberate), the ε-amine of the lysine (K) side chain was modified with Bis-PEG₄-TFP (e.g., from Vector Laboratories, Newark, CA; Catalog QBD-10085), to create the TFP Probe Reactive Moiety (Y1) on the PC-MT Labeling Reagent shown in FIG. 6 (Structure 1).

In this Example, the peptide-based TFP-activated PC-MT Labeling Reagent is referred to as PC-MT-TFP.

### Covalent Conjugation of PC-MT-TFP to Antibody Probes to Create PC-MT Labeled Antibody Probes (PC-MT-Antibodies)

Performed as in Example 1 except using the PC-MT-TFP reagent at a 15-fold molar excess relative to the antibody instead of using PC-MT-NHS at a 10-fold molar excess. Moreover, the reaction time of the antibody with the PC-MT-TFP was overnight instead of 1 hr. An example structure of a PC-MT-antibody (PC-MT Labeled Probe) created with this approach is shown in FIG. 6, Structure 3 (note that while one attached PC-MT is shown in FIG. 6 for simplicity, this procedure generally creates multiple attached PC-MTs, e.g., 3-5, on the antibody by PC-MT-TFP conjugation to available primary amines in the antibody).

### MALDI-IHC on FFPE Tonsil Tissue

The entire process was performed as in Example 1 except the PC-MT-antibody was only an anti-CD20 antibody (see Table II) and the optional Post-Fixation was not performed.

TABLE II
Variable Peptide Sequences used in the Mass-Tag (M) Component of PC-MT
Labeling Reagents and PC-MT Labeled (Antibody) Probes.

| | Amino Acid | Mass Reporter Mass | Antibody | Antibody |
|---|---|---|---|---|
| Name | Sequence* | (Monoisotopic) | Target | Clone |
| PC-MT- | (AP)R(L)R(F)YSL | 1,234.8606 | Collagen-1A1 | E8F4L |
| Iso1.06 | (SEQ ID NO: 1) | | | |
| PC-MT-7.13 | SGSLRRASLGGSG | 1,288.7080 | Pan- | C11 |
| | (SEQ ID NO: 13) | | Cytokeratin | |
| PC-MT- | A(P)R(L)R(F)YSL | 1,230.8406 | Vimentin | D21H3 |
| Iso1.05 | (SEQ ID NO: 2) | | | |
| PC-MT-1.04 | SAPRLRFYSLG | 1,350.7640 | CD8a | D8A8Y |
| | (SEQ ID NO: 3) | | | |
| PC-MT- | (AP)RLRFYSL | 1,216.7406 | CD68 | D4B9C |
| Iso1.02 | (SEQ ID NO: 14) | | | |
| PC-MT-1.03 | GAPRLRFYSLG | 1,320.7535 | Ki67 | 8D5 |
| | (SEQ ID NO: 4) | | | |
| PC-MT-15.00 | RGYGYQGL | 997.5214 | CD20 | E7B7T |
| | (SEQ ID NO: 5) | | | |

*Single-letter amino acid code is used. Amino acids in parenthesis are stable isotopic amino acids as defined in Table III.

TABLE III
Amino Acid Isotopes.

| 1-Letter* | Isotopic Amino Acid | FMOC-Isotopic Amino Acid |
|---|---|---|
| A | L-ALANINE (2,3,3,3-D4, 98%) | L-ALANINE-N-FMOC (2,3,3,3-D4, 98%) |
| P | L-PROLINE (13C5, 99%; 15N, 99%) | L-PROLINE-N-FMOC (13C5, 99%; 15N, 99%) |
| L | L-LEUCINE (D10, 98%) | L-LEUCINE-N-FMOC (D10, 98%) |
| F | L-PHENYLALANINE (D8, 98%) | L-PHENYLALANINE-N-FMOC (D8, 98%) |

*Single-letter amino acid code is used.

### Results

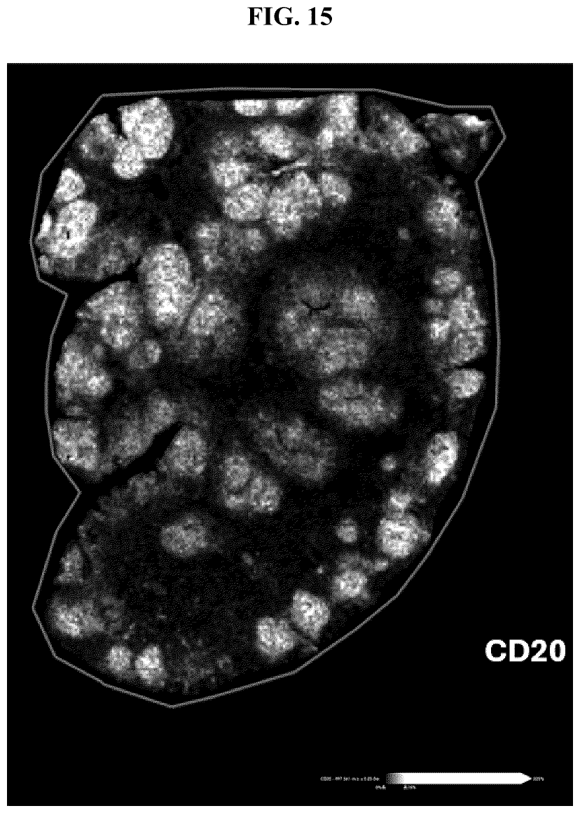

FIG. 15 shows the MALDI-IHC image of CD20. The CD20 antibody pattern matches the expected histology for a human tonsil based on the Human Protein Atlas database [97], that is, primarily germinal center staining.

### Example 3. A Maleimide-Activated Peptide-Based PC-MT Labeling Reagent Will be Created and Will be Used to Label an Antibody Probe Modified by Mild Reduction, Followed by MALDI-IHC Using the Probe

### Synthesis of the Maleimide-Activated Peptide-Based PC-MT Labeling Reagent

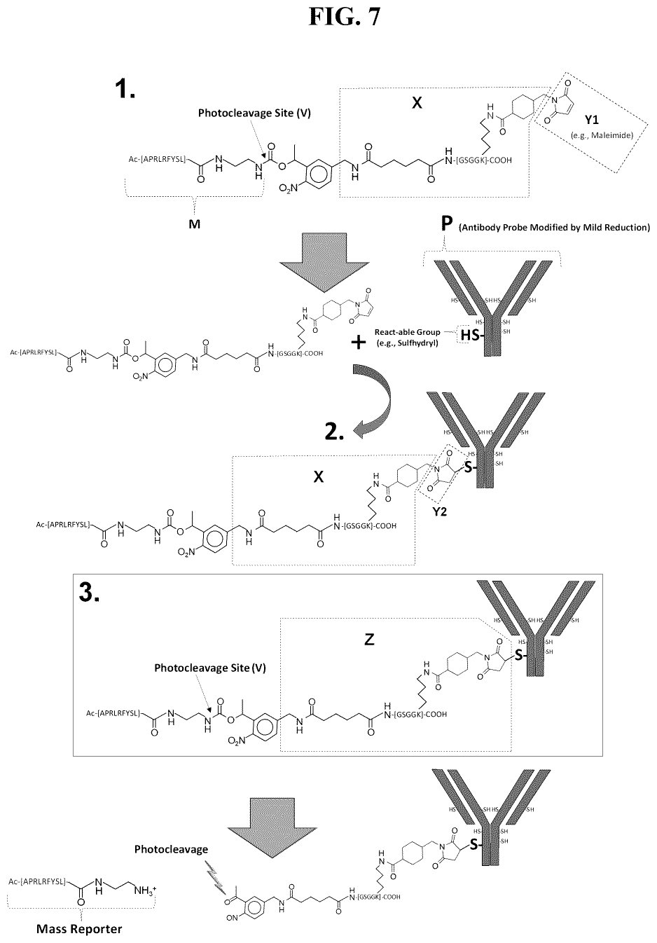

Will be performed as in Example 1, except that instead of generating an NHS-ester on the ε-amine of the lysine (K) side chain in the PC-MT Labeling Reagent using DSS (disuccinimidyl suberate), the ε-amine of the lysine (K) side chain will be modified using the SMCC reagent which is a heterobifunctional maleimide/NHS linker (e.g., Thermo Scientific, Waltham, MA; Catalog 22360), to create the maleimide Probe Reactive Moiety (Y1) on the PC-MT Labeling Reagent shown in FIG. 7 (Structure 1).

In this Example, the peptide-based maleimide-activated PC-MT Labeling Reagent is referred to as PC-MT-Maleimide.

### Covalent Conjugation of PC-MT-Maleimide to Antibody Probes to Create PC-MT Labeled Antibody Probes (PC-MT-Antibodies).

First, the source antibodies for labeling (same source antibodies as in Example 1) will be prepared in PBS at 1 mg/mL (with no other proteins or buffer constituents). The antibodies will then be modified by mild reduction for 30 min by addition of a 10-fold molar excess of TCEP (e.g., from Thermo Scientific, Waltham, MA) from a 10× stock prepared fresh in water. TCEP will then be removed from the antibodies using the diafiltration method described in Example 1, except PBS will be used as the diluent in this case and the 2× Storage Buffer will not be added at this stage. Buffers purged with nitrogen gas will be used to minimize reformation of the disulfide bonds from the TCEP-reduced sulfhydryls on the antibodies. The antibodies, now modified by mild reduction and in PBS, will then be conjugated to the PC-MT-Maleimide as in Example 1, except sodium bicarbonate and quenching reagent (glycine) will not be used (but 2× Storage Buffer will be added at the last step as in Example 1). An example structure of a PC-MT-antibody (PC-MT Labeled Probe) that will be created with this approach is shown in FIG. 7, Structure 3 (note that while one attached PC-MT is shown in FIG. 7 for simplicity, this procedure will generally create multiple attached PC-MTs, e.g., 3-5, on the antibody by PC-MT-Maleimide conjugation to available sulfhydryls in the antibody).

### MALDI-IHC on FFPE Tonsil Tissue

The entire process was performed as in Example 1 except the PC-MT-antibodies prepared as detailed earlier in this Example.

### Results

It is anticipated that the expected MALDI-IHC staining pattern and histology will be observed as described in the Results section of Example 1 (e.g., similar to that in FIG. 14A-C).

### Example 4. MALDI Mass Spectrometry Based In Situ Hybridization (MALDI-ISH) on Fresh Frozen (FF) Tissue Sections Using Photocleavable Mass-Tagged Oligo Probes Created by Copper-Free Click Chemistry

### Synthesis of the Azide-Activated Peptide-Based PC-MT Labeling Reagent

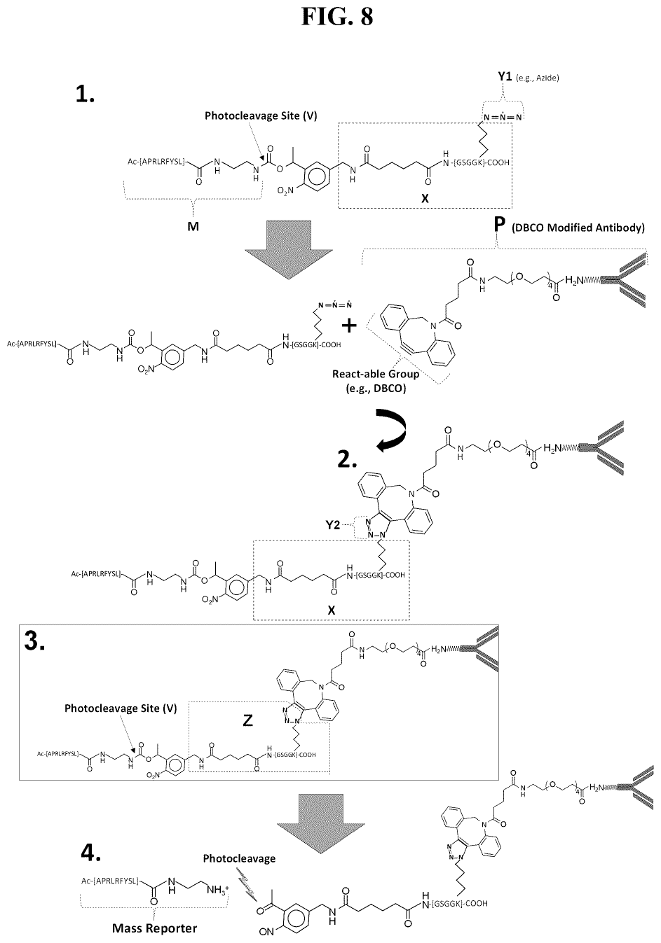

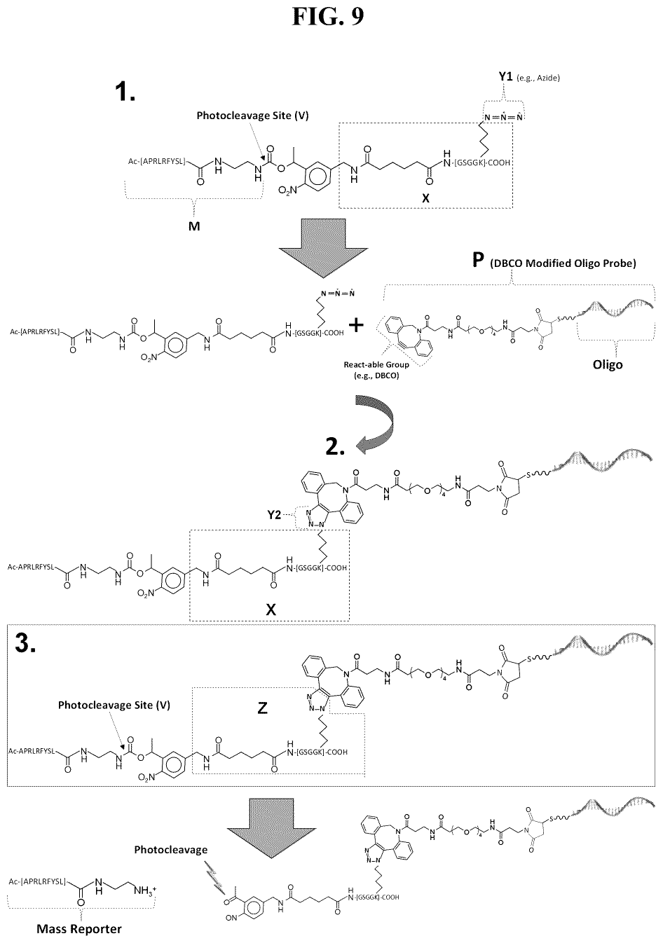

Performed as in Example 1, except that instead of generating an NHS-ester on the ε-amine of the lysine (K) side chain in the PC-MT Labeling Reagent using DSS (disuccinimidyl suberate), an azido lysine (e.g., using Fmoc-Azidolysine from Sigma-Millipore, St. Louis, MO; Catalog 8.52326) was incorporated at the lysine position using standard Fmoc SPPS methods, instead of a regular lysine, to create the Azide Probe Reactive Moiety (Y1) on the PC-MT Labeling Reagent shown in FIG. 8 and FIG. 9 (Structure 1).

In this Example, the peptide-based Azide-activated PC-MT Labeling Reagent is referred to as PC-MT-Azide.

### Covalent Conjugation of PC-MT-Azide to Oligonucleotide Probes to Create PC-MT Labeled Oligo Probes (PC-MT-Oligos)

As the starting oligonucleotide (oligo) material for labeling with PC-MTs, “RNAscope® HiPlex metal-ready Probes” were custom ordered from Biotechne/Advanced Cell Diagnostics (ACD) (Newark, CA), which correspond to the so-called T1-T12 detector oligos for 12-plex RNAscope assays (RNAscope detailed later in this Example; see Table IV for oligos and PC-MT assignments). These “label-ready” oligo Probes contain a disulfide group (e.g., incorporated during oligo synthesis using the “5′ Thiol Modifier C6 S—S”, Modification Code/5ThioMC6-D/, from Integrated DNA Technologies [IDT], Coralville, Iowa) which when reduced, results in a terminal thiol/sulfhydryl group. These oligos were conjugated to PC-MTs as follows:

The lyophilized oligos were resuspended in 80 μL water to a final concentration of 250 μM in 1.5 mL micro-centrifuge tubes. Concentration was confirmed by absorbance at 260 nm in a Nanodrop One^{C} microvolume spectrophotometer (Thermo Fisher Scientific, Waltham, MA). 50 μL of the 250 μM oligos was mixed with 5 μL of 0.5 M Bond-Breaker™ TCEP Solution (Thermo Scientific, Waltham, MA) and reacted for 30 min with gentle mixing to reduce the disulfide group on the oligos to generate a terminal thiol/sulfhydryl group.

Next, ethanol precipitation was performed to purify the oligos as follows: 5.5 μL of 3M sodium acetate (final concentration 0.3 M) was added to the oligos and mixed briefly by vortex. 150 μL (2.5 volumes) of 100% EtOH (pre-chilled to −20° C.) was then added and mixed briefly by vortex. The solutions were placed into a −20° C. freezer for at least 30 min. The oligos were spun down for at least 30 minutes at maximum speed in a standard refrigerated micro-centrifuge (˜15,000×g). The supernatant was then removed, being careful not to disturb the oligo pellet. 500 μL ice-cold 70% ethanol was gently added to wash the pellet and the centrifugation was repeated but for only 3 min. The supernatant was again removed, being careful not to disturb the pellet. The tubes were then left with the lids open to briefly air-dry the pellets (1-3 min).

Oligo pellets were then resuspended in 100 μL of PBS and the concentration determined by absorbance at 260 nm on a Nanodrop One^{C} microvolume spectrophotometer. Based on this measurement, the oligos were diluted to 50 μM in PBS. 3.9 mM EZ-Link™ Maleimide-PEG4-DBCO (Thermo Scientific, Waltham, MA) was freshly prepared by dissolving 1 mg (MW 647.74) in 400 μL of anhydrous DMF. 52 μL of the 3.9 mM EZ-Link™ Maleimide-PEG4-DBCO solution (200 nmoles) was added to the 50 μM oligo solutions followed by mixing. The reaction was carried out for 2 hrs with gentle mixing. The resultant DBCO Modified Oligo Probe is shown as P in FIG. 9.

Without purification, the DBCO Modified Oligo Probes were then reacted with the PC-MT-Azide reagent. To do so, a 10 mM stock of PC-MT-Azide reagent was prepared in anhydrous DMF. 30 μL of the 10 mM PC-MT-Azide (300 nmoles) was then added to the DBCO Modified Oligo Probes. The reaction was carried out for overnight protected from light and with gentle mixing.

Removal of unreacted PC-MT-Azide and buffer exchange was then achieved using NAP-5 G-25 Sephadex columns (Cytiva Life Sciences, Marlborough, MA) per the manufacturer's instructions, against TE-150 mM NaCl buffer (TE [10 mM Tris, pH 8.0, 1 mM EDTA] with 150 mM NaCl): Briefly, the column top and bottom caps were removed and the storage buffer allowed to flow through to waste. The columns were then pre-equilibrated with 3 full column fillings of TE-150 mM NaCl (flow-through to waste). This was ˜10 mL total of pre-equilibration buffer volume. When the last of the pre-equilibration buffer had fully entered the column, the oligo samples were added and the flow through discarded to waste. After the sample had fully entered the column, 190 μL (or enough to total 500 μL loaded) of TE-150 mM NaCl was added and the flow-through again discarded to waste. Finally, after the added TE-150 mM NaCl had fully entered the column, the sample was eluted with ˜0.7 mL TE-150 mM NaCl, with the flow-through (eluate) collected this time into a clean 1.5 mL micro-centrifuge tube. The oligo concentration was determined by absorbance at 260 nm on a Nanodrop One^{C} microvolume spectrophotometer.

The PC-MT conjugated oligo Probes were further purified by ethanol precipitation in batch sizes of 300 μL of the aforementioned NAP-5 eluate as follows: 33.33 μL of 3M sodium acetate (final concentration 0.3 M) was added to the oligos and mixed briefly by vortex. 840 μL (2.5 volumes) of 100% EtOH (pre-chilled to −20° C.) was then added and mixed briefly by vortex. The solutions were placed into a −20° C. freezer for at least 30 min. The oligos were spun down for at least 30 minutes at maximum speed in a standard refrigerated micro-centrifuge (˜15,000×g). The supernatant was then removed, being careful not to disturb the oligo pellet. 500 μL ice-cold 70% ethanol was gently added to wash the pellet and the centrifugation was repeated but for only 3 min. The supernatant was again removed, being careful not to disturb the pellet. The tubes were then left with the lids open to briefly air-dry the pellets (1-3 min). The final PC-MT conjugated oligo Probes (PC-MT-oligos) were resuspended in 100 μL of TE Buffer (10 mM Tris, pH 8.0, 1 mM EDTA) and the oligo concentration was determined by absorbance at 260 nm on a Nanodrop One^{C} microvolume spectrophotometer.

Conjugation of the PCMTs to the oligos was confirmed using the E-Gel Power Snap Electrophoresis System using pre-cast E-gels with SYBR Safe DNA stain according to the manufacturer's instructions (Thermo Scientific, Waltham, MA).

An example structure of a PC-MT-oligo (PC-MT Labeled Probe) created with this approach is shown in FIG. 9, Structure 3.

TABLE IV
Variable Peptide Sequences used in the Mass-Tag (M) Component of PC-MT
Labeling Reagents and PC-MT Labeled (Oligo) Probes for Examples 4-5.

| | | | | Corresponding |
|---|---|---|---|---|
| | Amino Acid | Mass Reporter | | Transcript- |
| | Sequence* | Mass | RNAscope | Specific Z-Probe |
| Name | (SEQ ID NO:) | (Monoisotopic) | Oligo Tail | (Mouse Reactive) |
| PC-MT-9.01 | RYPFPGPG | 974.5206 | T1 | TMEM119 |
| | (SEQ ID NO: 6) | | | |
| PC-MT-1.00 | APRLRFYSL | 1206.7106 | T2 | Alpha-Synuclein |
| | (SEQ ID NO: 7) | | | |
| PC-MT-2.05 | GRPPGFSFFRGG | 1365.7174 | T3 | Parvalbumin |
| | (SEQ ID NO: 8) | | | |
| PC-MT-1.02 | SAPRLRFYSL | 1293.7426 | T4 | GFAP |
| | (SEQ ID NO: 9) | | | |
| PC-MT-14.03 | GPPGFSPFRG | 1102.5792 | T5 | Cadherin |
| | (SEQ ID NO: 10) | | | |
| PC-MT-2.00 | RPPGFSFFR | 1194.653 | T6 | APP |
| | (SEQ ID NO: 11) | | | |
| PC-MT-7.11 | SGLRRASLGGSG | 1201.676 | T7 | MBP |
| | (SEQ ID NO: 12) | | | |
| PC-MT-7.13 | SGSLRRASLGGSG | 1288.708 | T8 | Cathepsin D |
| | (SEQ ID NO: 13) | | | |
| PC-MT- | (AP)RLRFYSL | 1216.7406 | T9 | Synapsin-I |
| Iso1.02 | (SEQ ID NO: 14) | | | |
| PC-MT-7.14 | GSGSLRRASLGGSG | 1345.7295 | T10 | Tau |
| | (SEQ ID NO: 15) | | | |
| PC-MT-1.05 | GAPRLRFYSLGG | 1377.7749 | T11 | NeuN |
| | (SEQ ID NO: 16) | | | |
| PC-MT-14.09 | SGPPGFSPFRGS | 1276.6433 | T12 | Tubulin Beta 3 |
| | (SEQ ID NO: 17) | | | |

*Single-letter amino acid code is used. Amino acids in parenthesis are stable isotopic amino acids as defined in Table III.

### MALDI Mass Spectrometry Based In Situ Hybridization (MALDI-ISH) Using the PC-MT-Oligos on Fresh Frozen (FF) Tissue Sections

Overall, the approach in this Example was based on the RNAscope RNA in situ hybridization technology [98] (now commercialized by Advanced Cell Diagnostics [ACD]/BioTechne, Newark, CA). RNAscope uses the following basic steps in sequence: Hybridization of complementary transcript-specific oligonucleotide (oligo) Probes to the targets in the cell/tissue sample; these oligo Probes have extra sequences, so-called “tails”, which are not complementary to the target sequence, which mediate the next hybridization step, which is hybridization of pre-amplifier oligos (which again have their own tails) to the tails in the transcript-specific Probes, and then hybridization of many amplifier oligos to the tails in the pre-amplifiers, a form of branched DNA amplification (bDNA). Finally, hybridization of the labeled oligo Probes (detector Probes) to the multiple complementary sites generated by the bDNA steps is performed. RNAscope currently offers 12 tails, T1-T12 in the “HiPlex” assay format. It is to be understood that other amplification methodologies are possible, such as SABER [49, 99]. Direct hybridization to the cell/tissue targets (e.g., targets such as mRNA transcripts, miRNAs, or antisense oligo drugs [ASOs]) of complementary PC-MT-oligos carrying one or more PC-MTs is also possible (e.g., U.S. Pat. No. 11,906,527 which is hereby incorporated by reference). Overall, RNAscope, SABER, and fluorescence in situ hybridization (FISH) were developed for optical imaging of fluorescent and chromogenic detector oligo Probes, whereas in the Present Invention, PC-MT labeled oligo Probes (prepared as detailed earlier in this Example) are used and combined with non-optical MALDI-MSI as follows:

The HiPlex RNAscope assay was essentially performed according to the manufacturer's instructions for fresh frozen (FF) tissue sections (Advanced Cell Diagnostics [ACD]/BioTechne, Newark, CA), except the PC-MT-oligos were used instead of fluorescent oligos at the detector Probe step and the final washes were modified to remove salts which are incompatible with MALDI-MSI. The detailed protocol was as follows:

hAbeta^{SAA} (APP-SAA) fresh frozen (FF) mouse brain tissue sections (see Materials) were used as the sample, and the 10 μm cryosections were mounted on Bruker IntelliSlides™ (see Materials).

FF tissue slides were removed from −80° C. storage and immediately immersed in 4% PFA in PBS fixative (prepared as in Example 1) in a polypropylene Coplin jar for 60 min. Unless otherwise noted, all bulk volume steps were in Coplin jars using an excess (˜70 mL) of solution. Slides were washed 2×2 min each in PBS. Tissues were then dehydrated with an aqueous/ethanol series as follows (5 min each): 50% ethanol (v/v in water), 70% ethanol, and 2× with 100% ethanol. Slides were air dried for 5 min at 60° C. in a laboratory oven.

Note, when indicated by “Intro Pack”, the reagents were from the commercial RNAscope™ Intro Pack for HiPlex12 Reagents Kit (ACD, Catalog 324442).

Tissue sections were then each surround by hydrophobic barrier pen markings to facilitate the following small volume reagent incubations (i.e., 100 μL/cm²; not performed in Coplin jars unless otherwise noted): Protease IV (Intro Pack), warmed to room temperature or 40° C., was incubated with the tissue for 30 min at 40° C. in a humidified chamber. Slides were rinsed 2× briefly with PBS (˜70 mL in Coplin Jar). The 12-plex mixture of transcript-specific Z-Probes (ACD), each diluted 1/50 in RNAscope HiPlex Probe Diluent (Intro Pack), was applied to one of the tissue sections on the slide (see Table IV for Z-Probes, which were obtained from ACD against various neurologically relevant targets). To a second, serial tissue section on the slide, a 12-plex negative control mixture of Z-Probes (Intro Pack), diluted in the same manner, was added (these were Z-Probes targeting irrelevant bacterial sequences absent in the mouse brain tissue sample, but comprising the same amplification “tails” T1-12). Slides were incubated in a humidified chamber for 2 hrs at 40° C. to allow for Z-Probe hybridization to their targets.

The following steps used 10 cm glass petri dishes and ˜20 mL of solution per step: Slides were washed with gentle mixing 2×2 min each with 1× Wash Buffer, diluted with nuclease-free water (Promega, Madison, WI) from 50× Wash Buffer (Intro Pack). Slides were stored overnight in 5×SSC (750 mM NaCl and 75 mM sodium citrate, pH 7.0). The next day, slides were washed 2×2 min each with 1× Wash Buffer (Intro Pack).

The following small volume reagent incubations were performed within the hydrophobic barrier pen markings (i.e., 100 μL/cm² unless otherwise noted): Slides were incubated at 40° C. for 30 min with HiPlex Amp 1 (Intro Pack) in a humidified chamber. Slides were then washed 2×2 min with 1× Wash Buffer (˜20 mL in petri dish). Slides were incubated at 40° C. for 30 min with HiPlex Amp 2 (Intro Pack) in a humidified chamber. Slides were then washed 2×2 min with 1× Wash Buffer (˜20 mL in petri dish). Slides were incubated at 40° C. for 30 min with HiPlex Amp 3 (Intro Pack) in a humidified chamber. Slides were then washed 2×2 min with 1× Wash Buffer (˜20 mL in petri dish). Slides were incubated at 40° C. for 30 min with a mixture of the 12 PC-MT-oligos (for T1-12; 20 nM each diluted in RNAscope HiPlex Probe Diluent) in a humidified chamber. Slides were then washed 2×2 min with 1× Wash Buffer (˜20 mL in petri dish).

The final washes to remove salts which are incompatible with MALDI-MSI were as follows: Rinse briefly for 10 seconds then 3×2 min, with excess 50 mM ammonium bicarbonate at each step, in a 10 cm glass petri dish with gentle shaking. Dry the slides for 1.5 hrs in a vacuum desiccation chamber.

PC-MT photocleavage, matrix application and MALDI-MSI were performed as in Example 1.

### Results

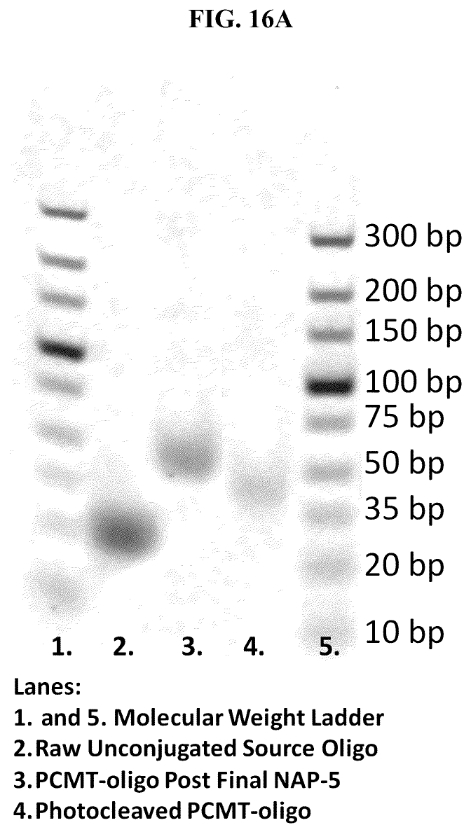

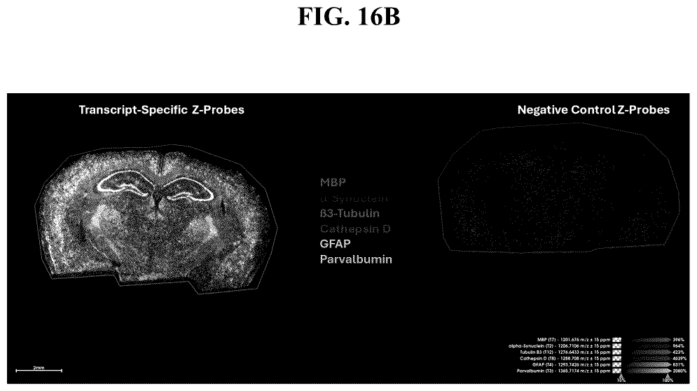

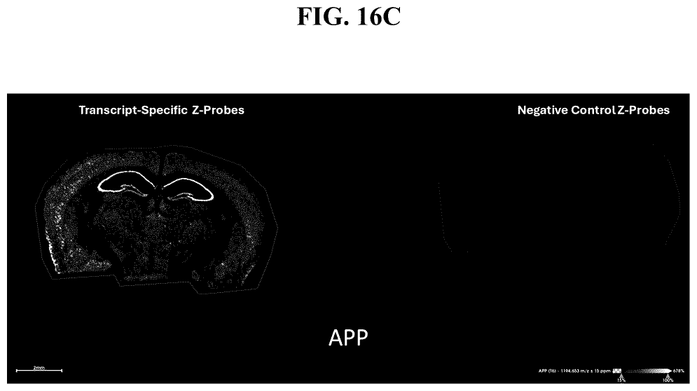

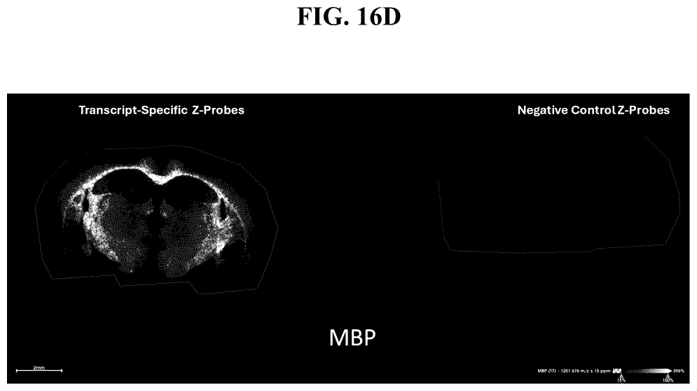

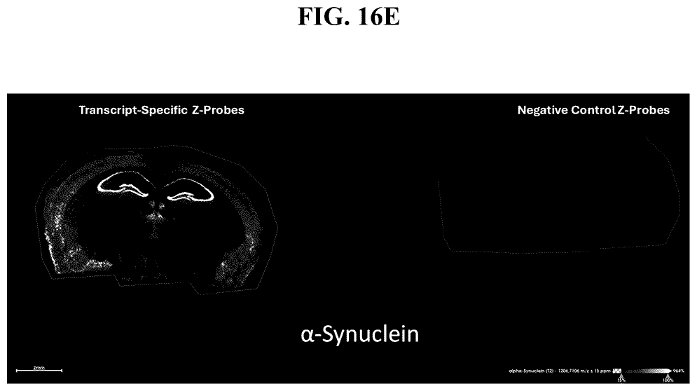

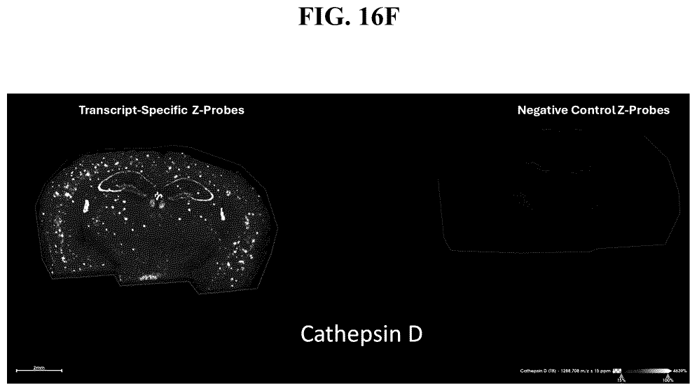

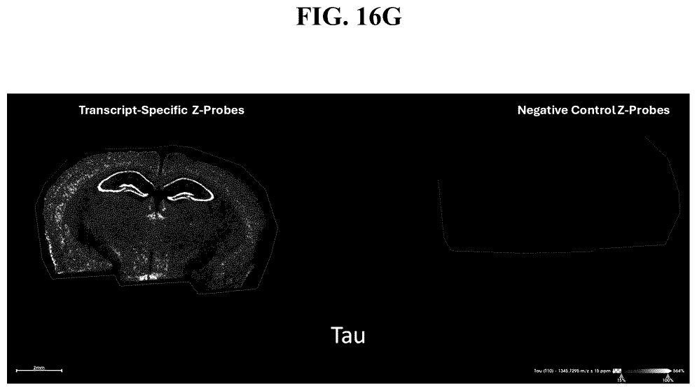

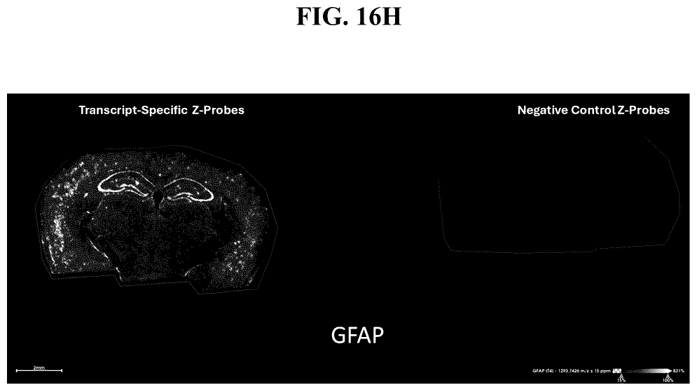

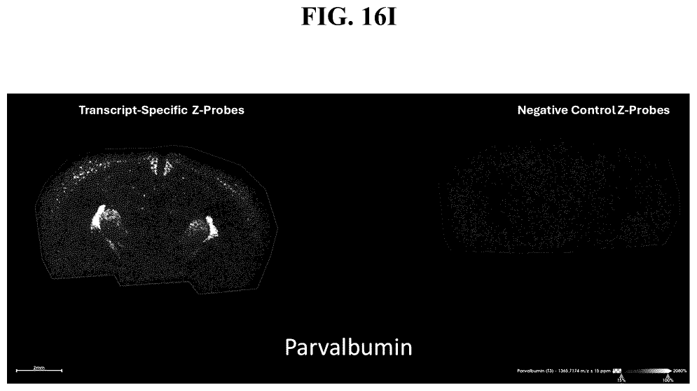

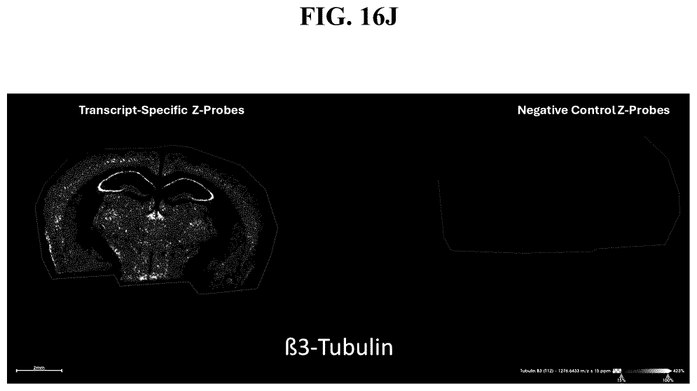

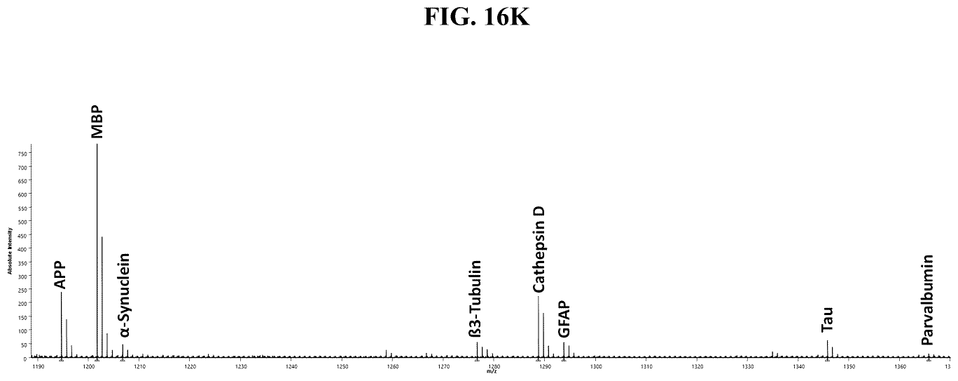

Results are shown in FIG. 16A-J. FIG. 16A shows example results of gel electrophoresis analysis on one example PC-MT-oligo, whereby the raw unconjugated source oligo (˜20 bp) runs at the expected position (Lane 2). Following PC-MT conjugation to the oligo and final purification by NAP-5 column chromatography (Lane 3), a clear increase in mass in observed (running approximately equivalent to 60 bp), with no remaining raw unconjugated source oligo detected, indicating efficient PC-MT conjugation. Following photocleavage of the PC-MT-oligo, the mass is reduced (Lane 4), however, due to the oligo-conjugated Maleimide-PEG4-DBCO linker and portions of the subsequently-conjugated PC-MT Labeling Reagent that are not photocleaved (see FIG. 9, bottommost structures), the mass is not fully reduced to the original raw unconjugated oligo. Instead, upon photocleavage, an intermediate mass, between the original raw unconjugated source oligo and the non-photocleaved PC-MT-oligo is observed (running approximately equivalent to 40 bp), as expected.

FIG. 16B shows the spatial map of the relative intensities of the PC-MT Mass Reporters (hereafter referred to as MALDI-ISH images) corresponding to 6 of the PC-MT-oligos shown as an example. The left tissue section (Test Sample) shown was treated with the transcript-specific Z-Probes (see Table IV) show specific signal as expected. Conversely, the right tissue section, (Negative Control) was treated with Z-Probes against bacterial sequences not present in mammals, whereby the Z-Probes contain the same “tails” T1-T12, and whereby this tissue section was otherwise subjected to the same procedure as the Test Sample, including the PC-MT-oligos. The Negative Control tissue section shows no significant signal as expected. FIG. 16C-FIG. 16J show MALDI-ISH images of single PC-MT-oligos, corresponding to one particular transcript as indicated in the figures. FIG. 16K shows the overall average mass spectrum from the Test Sample (averaged from the entire tissue section), with the labels indicating the peaks for 8 different PC-MT reporter ions, corresponding to the detection of the specific transcripts indicated. A wide dynamic range is observed in the average spectrum, with MBP being the most abundant as expected, whereas Parvalbumin is more highly localized (see FIG. 16I), providing the lowest signal in the spectrum when averaged over the whole tissue section.

### Example 5. MALDI Mass Spectrometry Based In Situ Hybridization (MALDI-ISH) Using Photocleavable Mass-Tagged Oligo Probes on Formalin-Fixed Paraffin-Embedded (FFPE) Tissue Sections

### Synthesis of the Azide-Activated Peptide-Based PC-MT Labeling Reagent

Performed as in Example 4. In this Example, the peptide-based Azide-activated PC-MT Labeling Reagent is referred to as PC-MT-Azide.

### Covalent Conjugation of PC-MT-Azide to Oligonucleotide Probes to Create PC-MT Labeled Oligo Probes (PC-MT-Oligos)

Performed as in Example 4. An example structure of a PC-MT-oligo (PC-MT Labeled Probe) created with this approach is shown in FIG. 9, Structure 3.

### MALDI Mass Spectrometry Based In Situ Hybridization (MALDI-ISH) Using the PC-MT-Oligos on Formalin Fixed Paraffin Embedded (FFPE) Tissue Sections

Overall, the approach in this Example was based on the RNAscope RNA in situ hybridization technology [98](now commercialized by Advanced Cell Diagnostics [ACD]/BioTechne, Newark, CA) as detailed in Example 4.

The HiPlex RNAscope assay was essentially performed according to the manufacturer's instructions for formalin fixed paraffin embedded (FFPE) tissue sections (Advanced Cell Diagnostics [ACD]/BioTechne, Newark, CA), except the PC-MT-oligos were used instead of fluorescent oligos at the detector Probe step and the final washes were modified to remove salts which are incompatible with MALDI-MSI. The detailed protocol was as follows:

hAbeta^{SAA} (APP-SAA) FFPE mouse brain tissue sections (see Materials) were used as the sample, and the 3 μm microtome sections were mounted on Fisherbrand™ Superfrost™ Plus Microscope Slides (Thermo Scientific, Waltham, MA). Before use, slides were heated for 1 hr at 60° C. in a laboratory oven to promote tissue adhesion to the surface.

Unless otherwise noted, all bulk volume steps were in Coplin jars using an excess (˜70 mL) of solution. Slides were incubated 2×5 min each in xylene and 2×2 min each in ethanol and then dried in a laboratory oven for 5 min at 60° C.

Note, when indicated by “Intro Pack”, the reagents were from the commercial RNAscope™ Intro Pack for HiPlex12 Reagents Kit (ACD, Catalog 324442).

Incubate slides for 15 min at 96° C. in 1× Target Retrieval Reagent (Intro Pack) (performed using a Coplin jar placed in a water bath). Slides were then incubated for 15 s in molecular biology grade water (see Materials) and then 3 min in 100% ethanol, and then dried in a laboratory oven for 5 min at 60° C.

Tissue sections were then each surround by hydrophobic barrier pen markings (dried 10 min or more) to facilitate the following small volume reagent incubations (i.e., 100 μL/cm²; not performed in Coplin jars unless otherwise noted): Protease III (Intro Pack), warmed to room temperature or 40° C., was incubated with the tissue for 30 min at 40° C. in a humidified chamber. Slides were rinsed 2×15 s each with molecular biology grade water (˜70 mL in Coplin Jar). The 12-plex mixture of transcript-specific Z-Probes (ACD), each diluted 1/50 in RNAscope HiPlex Probe Diluent (Intro Pack), was applied to one of the tissue sections on the slide (see Table IV for Z-Probes, which were obtained from ACD against various neurologically relevant targets). To a second, serial tissue section on the slide, a 12-plex negative control mixture of Z-Probes (Intro Pack), diluted in the same manner, was added (these were Z-Probes targeting irrelevant bacterial sequences absent in the mouse brain tissue sample, but comprising the same amplification “tails” T1-12). Slides were incubated in a humidified chamber for 2 hrs at 40° C. to allow for Z-Probe hybridization to their targets.

The following steps used 10 cm glass petri dishes and ˜20 mL of solution per step: Slides were washed with gentle mixing 2×2 min each with 1× Wash Buffer, diluted with nuclease-free water (Promega, Madison, WI) from 50× Wash Buffer (Intro Pack). Slides were stored overnight in 5×SSC (750 mM NaCl and 75 mM sodium citrate, pH 7.0). The next day, slides were washed 2×2 min each with 1× Wash Buffer (Intro Pack).

The following small volume reagent incubations were performed within the hydrophobic barrier pen markings (i.e., 100 μL/cm² unless otherwise noted): Slides were incubated at 40° C. for 30 min with HiPlex Amp 1 (Intro Pack) in a humidified chamber. Slides were then washed 2×2 min with 1× Wash Buffer (˜20 mL in petri dish). Slides were incubated at 40° C. for 30 min with HiPlex Amp 2 (Intro Pack) in a humidified chamber. Slides were then washed 2×2 min with 1× Wash Buffer (˜20 mL in petri dish). Slides were incubated at 40° C. for 30 min with HiPlex Amp 3 (Intro Pack) in a humidified chamber. Slides were then washed 2×2 min with 1× Wash Buffer (˜20 mL in petri dish). Slides were incubated at 40° C. for 30 min with a mixture of the 12 PC-MT-oligos (for T1-12; 20 nM each diluted in RNAscope HiPlex Probe Diluent) in a humidified chamber. Slides were then washed 2×2 min with 1× Wash Buffer (˜20 mL in petri dish).

The final washes to remove salts which are incompatible with MALDI-MSI were as follows: Rinse briefly for 10 seconds then 3×2 min, with excess 50 mM ammonium bicarbonate at each step, in a 10 cm glass petri dish with gentle shaking. Dry the slides for 1.5 hrs in a vacuum desiccation chamber.

PC-MT photocleavage, matrix application and MALDI-MSI were performed as in Example 1.

### Results

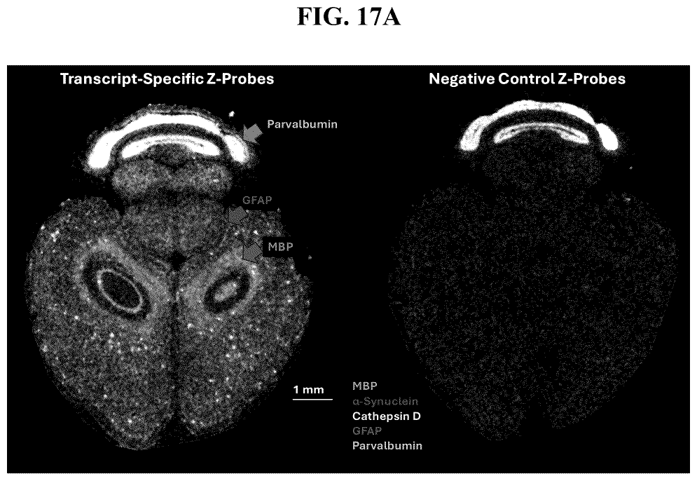

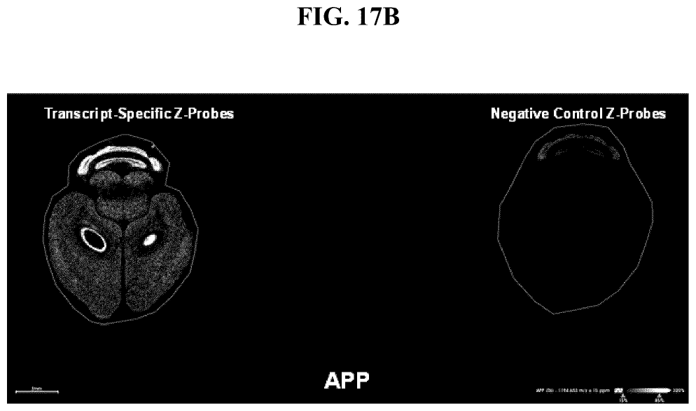

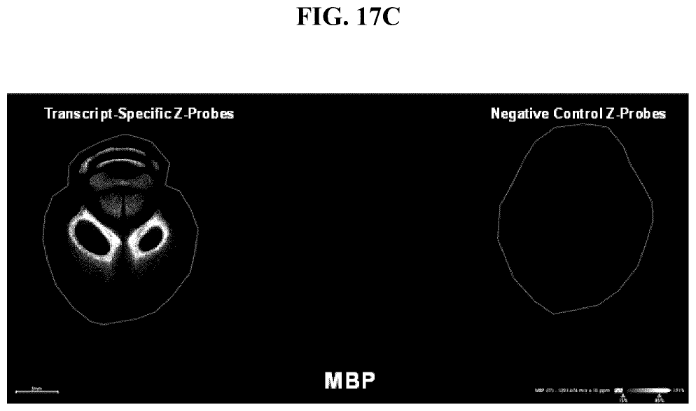

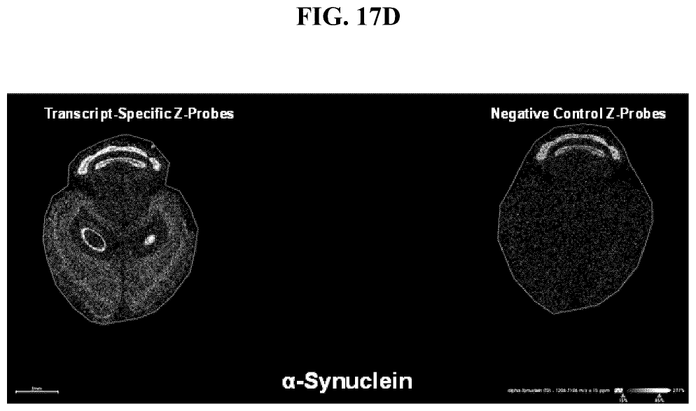

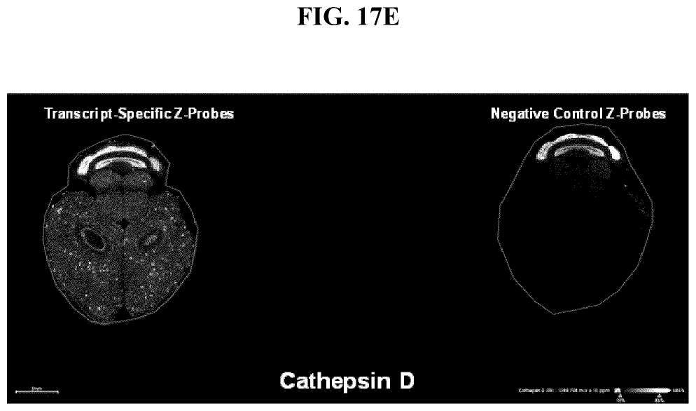

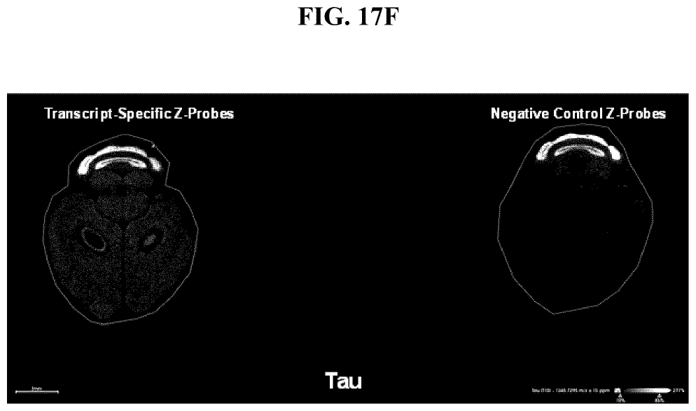

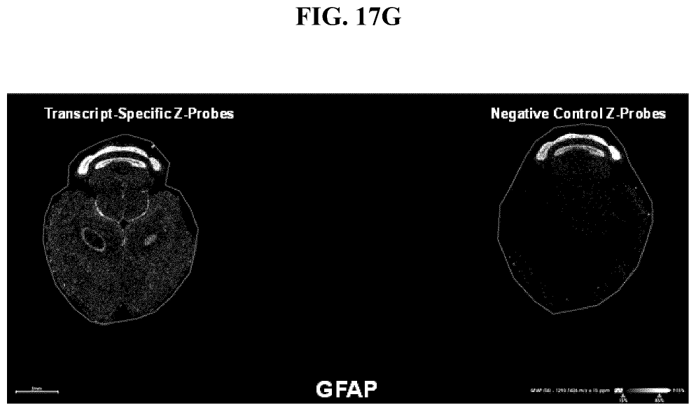

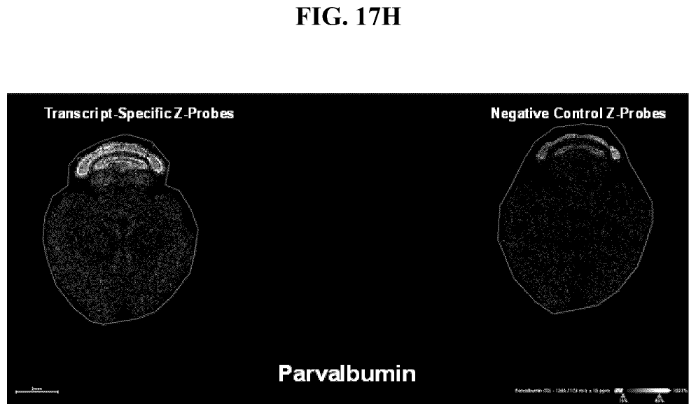

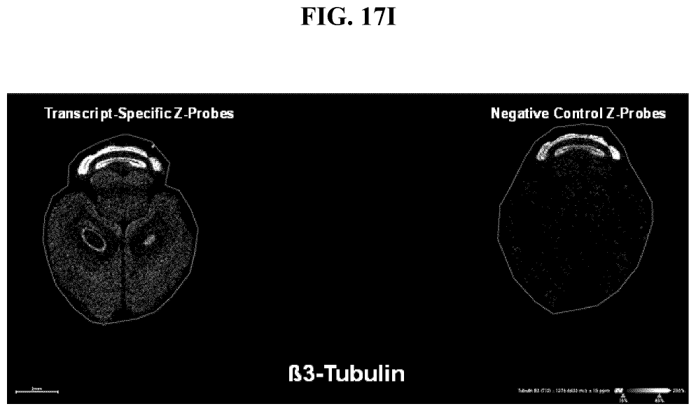

FIG. 17A shows the spatial map of the relative intensities of the PC-MT Mass Reporters (hereafter referred to as MALDI-ISH images) corresponding to 5 of the PC-MT-oligos shown as an example. The left tissue section (Test Sample) shown was treated with the transcript-specific Z-Probes (see Table IV) show specific signal as expected. Conversely, the right tissue section, (Negative Control) was treated with Z-Probes against bacterial sequences not present in mammals, whereby the Z-Probes contain the same “tails” T1-T12, and whereby this tissue section was otherwise subjected to the same procedure as the Test Sample, including the PC-MT-oligos. The Negative Control tissue section shows no significant signal with the exception of some non-specific Probe binding in the cerebellum. However, it is clear by comparison between the Test Sample and Negative Control that specific signals and staining patterns are observed in the Test Sample (the colored arrows in the Test Sample in FIG. 17A highlight some examples). FIG. 17B-FIG. 17I show MALDI-ISH images of single PC-MT-oligos, corresponding to one particular transcript as indicated in the figures.

### Example 6. Multiomics: Untargeted Small Molecule, MALDI-ISH and MALDI-IHC Imaging on the Same Fresh Frozen (FF) Tissue Section Using Photocleavable Mass-Tagged Oligo and Antibody Probes

Tissue samples were as in Example 4. Unprocessed fresh frozen (FF) tissue sections were first directly analyzed by MALDI-MSI for untargeted imaging of endogenous label-free lipids. This was performed after thawing the tissue sections in a vacuum desiccator and directly performing DAN matrix application to the dried tissue sections by sublimation followed by recrystallized according to published reports [37, 66]. MALDI-MSI was performed as in Example 1 (timsTOF fleX mass spectrometer in this case) except that negative ion-mode MALDI-MSI was used for lipid detection with the alkaline DAN matrix. Following MALDI-MSI of lipids, the remaining DAN matrix was removed, and the tissue simultaneously fixed by washing 2× with −80° C. acetone for 3 min each in Copling jars (˜70 mL) followed by drying the slides for 10 min in a vacuum desiccator.

Following the aforementioned, MALDI-ISH was performed according to Example 4 with the following exceptions:

Note, when indicated by “Intro Pack”, the reagents were from the commercial RNAscope™ Intro Pack for HiPlex12 Reagents Kit (Biotechne/Advanced Cell Diagnostics [ACD], Newark, CA, Catalog 324442).

The steps of surrounding the tissue sections with a hydrophobic barrier pen and Protease IV treatment in Example 4 were replaced with the following steps: Using a capped polypropylene Coplin jar (˜70 mL) the tissue slides were treated with 1× Target Retrieval Solution (Intro Pack) at 96° C. for 30 min. The following steps used 10 cm glass petri dishes and ˜20 mL of solution per step: Tissue slides were then rinsed briefly with LCMS grade water and washed 2×3 min each with 100% ethanol. Tissue slides were then air dried at 60° C. for 5 min. The hydrophobic barrier pen was then applied around the tissue sections to facilitate the following small volume incubations (˜100 μL/cm2 in a humidified chamber): PreTreat Pro solution (ACD), warmed to room temperature, was incubated on the tissue sections in the humidified chamber at 40° C. for 30 min.

The remaining MALDI-ISH steps were performed as in Example 4, starting after the Protease IV treatment described in Example 4 (note Protease IV step not performed in this Example). The PC-MT Probe panel and RNA targets are listed in Table V.

TABLE V
Variable Peptide Sequences used in the Mass-Tag (M) Component of PC-MT
Labeling Reagents and PC-MT Labeled (Oligo) Probes for Example 6.

| | | | | Corresponding |
|---|---|---|---|---|
| | | Mass Reporter | | Transcript- |
| | Amino Acid | Mass | RNAscope | Specific Z-Probe |
| Name | Sequence* | (Monoisotopic) | Oligo Tail | (Mouse Reactive) |
| PC-MT-9.01 | RYPFPGPG | 974.5206 | T1 | AIF1 (IBA-1) |
| | (SEQ ID NO: 6) | | | |
| PC-MT-1.00 | APRLRFYSL | 1206.7106 | T2 | Cadherin-5 |
| | (SEQ ID NO: 7) | | | |
| PC-MT-2.05 | GRPPGFSFFRGG | 1365.7174 | T3 | Parvalbumin |
| | (SEQ ID NO: 8) | | | |
| PC-MT-1.02 | SAPRLRFYSL | 1293.7426 | T4 | GFAP |
| | (SEQ ID NO: 9) | | | |
| PC-MT-14.03 | GPPGFSPFRG | 1102.5792 | T5 | GLUT1 (SLC2A1) |
| | (SEQ ID NO: 10) | | | |
| PC-MT-2.00 | RPPGFSFFR | 1194.653 | T6 | APP |
| | (SEQ ID NO: 11) | | | |
| PC-MT-7.11 | SGLRRASLGGSG | 1201.676 | T7 | Alpha-Synuclein |
| | (SEQ ID NO: 12) | | | |
| PC-MT-7.13 | SGSLRRASLGGSG | 1288.708 | T8 | Cathepsin D |
| | (SEQ ID NO: 13) | | | |
| PC-MT- | (AP)RLRFYSL | 1216.7406 | T9 | Tubulin Beta3 |
| Iso1.02 | (SEQ ID NO: 14) | | | |
| PC-MT-7.14 | GSGSLRRASLGGSG | 1345.7295 | T10 | NEFH |
| | (SEQ ID NO: 15) | | | |
| PC-MT-1.05 | GAPRLRFYSLGG | 1377.7749 | T11 | MBP |
| | (SEQ ID NO: 16) | | | |
| PC-MT-14.09 | SGPPGFSPFRGS | 1276.6433 | T12 | Tau |
| | (SEQ ID NO: 17) | | | |

* Single-letter amino acid code is used. Amino acids in parenthesis are stable isotopic amino acids
as defined in Table III.

After the MALDI-ISH imaging was completed, the matrix from the MALDI-ISH imaging was next removed by 2×3 min washes in 100% methanol using the 10 cm glass petri dishes and ˜20 mL of solution per step. MALDI-IHC was performed as in Example 1 except starting at the step of rehydration 2× with 10000 ethanol for 2 min each to the end of the protocol (omitting all steps prior to this start point). Antibodies and PC-MT assignments for the MALDI-IHC PC-MT-antibody panel used are shown in Table VI.

TABLE VI
Variable Peptide Sequences used in the Mass-Tag (M) Component of PC-MT
Labeling Reagents and PC-MT Labeled (Antibody) Probes for Example 6.

| | | Mass Reporter Mass | Antibody | |
|---|---|---|---|---|
| Name | Amino Acid Sequence* | (Monoisotopic) | Clone | Antibody Target |
| PC-MT- | GAPRLRFYSLY | 1,426.7953 | C67E7 | AKT (pan) |
| 1.16 | (SEQ ID NO: 18) | | | |
| PC-MT- | LRRASLG | 856.5475 | E4S6I | GLUT1 (SLC2A1) |
| 7.00 | (SEQ ID NO: 19) | | | |
| PC-MT- | GSGSRPPGFSFFRGSGS | 1,770.8670 | mOC64 | Amyloid-Beta42 |
| 2.15 | (SEQ ID NO: 20) | | | |
| PC-MT- | TSSAPRLRFYSLGSP | 1,722.9285 | E3F3P | APP |
| 1.18 | (SEQ ID NO: 21) | | | |
| PC-MT- | GPPGPSPFRG | 1,052.5636 | E7Z4L | Cathepsin D |
| 11.17 | (SEQ ID NO: 22) | | | |
| PC-MT- | RGYAYQGL | 1,011.5370 | E4L7M | GFAP |
| 16.00 | (SEQ ID NO: 23) | | | |
| PC-MT- | SGAPRLRFYSLGSGG | 1,608.8605 | D5C5Z | GSK-3Beta |
| 1.19 | (SEQ ID NO: 24) | | | |
| PC-MT- | (AP)R(L)RFYSL | 1,226.8106 | D17A3 | Histone H2A.X |
| Iso 1.04 | (SEQ ID NO: 25) | | | |
| PC-MT- | SLRRSSLG | 959.5745 | E404W | Iba-1 |
| 7.21 | (SEQ ID NO: 26) | | | |
| PC-MT- | SFLLRNPS | 1,017.5840 | D50G8 | LC3A |
| 10.16 | (SEQ ID NO: 27) | | | |
| PC-MT- | GRPPGFSFFRGG | 1,365.7174 | D8X4Q | MBP (Myelin Basic |
| 2.05 | (SEQ ID NO: 28) | | | Protein) |
| PC-MT- | GRPPGFSFFRG | 1,308.6960 | D4G40 | NeuN |
| 2.03 | (SEQ ID NO: 29) | | | |
| PC-MT- | GSGSLRRASLGGSG | 1,345.7295 | C28E10 | NF-L (Neurofilament |
| 7.14 | (SEQ ID NO: 15) | | | Light) |
| PC-MT- | PPGPSPFRT | 1,039.5683 | D4F6N | Nicastrin |
| 11.16 | (SEQ ID NO: 30) | | | |
| PC-MT- | SGAPRLRFYSLGSGW | 1,737.9183 | 5B3 | pGSK-3Beta (S9) |
| 1.20 | (SEQ ID NO: 31) | | | |
| PC-MT- | SGLRRASLGGSG | 1,201.6760 | D2Z4G | pTau-(pS404) |
| 7.11 | (SEQ ID NO: 12) | | | (Phospho Tau) |
| PC-MT- | YSAPRLRFYSLYQ | 1,747.9278 | E7D3E | pTau-(Thr205) |
| 1.23 | (SEQ ID NO: 32) | | | |
| PC-MT- | SGRPPGFSFFRGSG | 1,539.7815 | E8N2U | PVALB |
| 2.11 | (SEQ ID NO: 33) | | | (Parvalbumin) |
| PC-MT- | SFYLRNP | 980.5312 | E907E | Rab7 |
| 10.19 | (SEQ ID NO: 34) | | | |
| PC-MT- | SSSAPRLRFYSLSY | 1,717.9020 | D65A4 | Tubulin Beta3 |
| 1.28 | (SEQ ID NO: 35) | | | |
| PC-MT- | SGRPPGFSFFRGS | 1,482.7600 | D12G5 | Syanpsin I |
| 2.09 | (SEQ ID NO: 36) | | | |
| PC-MT- | TSAPRLRFYSLGSY | 1,701.9071 | Syn205 | Alpha/Beta-Synuclein |
| 1.26 | (SEQ ID NO: 37) | | | |

*Single-letter amino acid code is used. Amino acids in parenthesis are stable isotopic amino acids as defined in Table III.

### Results:

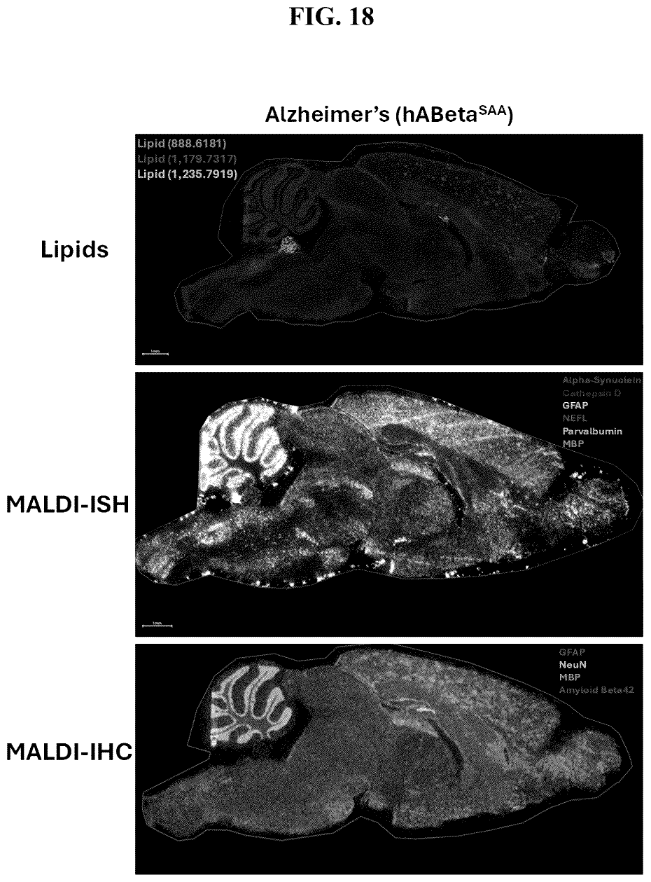

Results are shown in FIG. 18 for this tri-omic MALDI-MSI experiment on the same fresh frozen (FF) hABeta^{SAA} mouse brain tissue section. The MALDI-MSI images shown from the same tissue section indicate that i) untargeted label-free endogenous lipid imaging (“lipidomics”), ii) MALDI-ISH (“transcriptomics”) and iii) MALDI-IHC (“proteomics”) were successfully performed on the same tissue section using the same mass spectrometry instrument.

Thus, specific compositions and methods of Multiomic Mass Spectrometric Imaging of Tissues using Photocleavable Mass-Tag Probes have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES

1. Tang, L. (2023) “Spatially Resolved Multiomics,” Nature Methods 20(12), 1871.
2. Conrad, T. and Altmuller, J. (2022) “Single Cell- and Spatial 'Omics Revolutionize Physiology,” Acta Physiol (Oxf) 235(4), e13848.
3. Marx, V. (2021) “Method of the Year: Spatially Resolved Transcriptomics,” Nature Methods 18(1), 9-14.
4. Lewis, S. M. et al. (2021) “Spatial Omics and Multiplexed Imaging to Explore Cancer Biology,” Nature Methods 18(9), 997-1012.
5. Schueder, F. et al. (2020) “DNA-Barcoded Fluorescence Microscopy for Spatial Omics,” Proteomics 20(23), e1900368.
6. Howat, W. and Warford, A. (2014) “Advancing the Boundaries of Molecular Cellular Pathology,” Methods 70(1), 1-2.
7. Stack, E. C. et al. (2014) “Multiplexed Immunohistochemistry, Imaging, and Quantitation: A Review, with an Assessment of Tyramide Signal Amplification, Multispectral Imaging and Multiplex Analysis,” Methods 70(1), 46-58.
8. Katikireddy, K. R. and O'Sullivan, F. (2011) “Immunohistochemical and Immunofluorescence Procedures for Protein Analysis,” Methods Mol Biol 784, 155-167.
9. Dugger, B. N. and Dickson, D. W. (2017) “Pathology of Neurodegenerative Diseases,” Cold Spring Harb Perspect Biol 9(7).
10. Deng, H. X. et al. (2011) “Detection of Protein Aggregation in Neurodegenerative Diseases,” Methods Mol Biol 793, 259-272.
11. Zaha, D. C. (2014) “Significance of Immunohistochemistry in Breast Cancer,” World J Clin Oncol 5(3), 382-392.
12. Renwick, N. et al. (2013) “Multicolor Microrna FISH Effectively Differentiates Tumor Types,” J. Clin. Invest. 123(6), 2694-2702.
13. Halse, H. et al. (2018) “Multiplex Immunohistochemistry Accurately Defines the Immune Context of Metastatic Melanoma,” Sci Rep 8(1), 11158.
14. Geethamala, K. et al. (2017) “Comparison of Immunocytochemistry and Immunohistochemistry on Breast Carcinoma: A Boon or a Bane?,” J Lab Physicians 9(1), 5-10.
15. Russo, J. and Russo, I. H. (1986) “Immunocytochemistry as a Tool in Cellular, Molecular Biology, and Pathology: A Review,” Microsc. Electron. Biol. Celular 10(1), 1-63.
16. Blom, S. et al. (2017) “Systems Pathology by Multiplexed Immunohistochemistry and Whole-Slide Digital Image Analysis,” Sci Rep 7(1), 15580.
17. Renwick, N. et al. (2014) “Multiplexed Mirna Fluorescence in Situ Hybridization for Formalin-Fixed Paraffin-Embedded Tissues,” Methods Mol Biol 1211, 171-187.
18. Gorris, M. A. J. et al. (2018) “Eight-Color Multiplex Immunohistochemistry for Simultaneous Detection of Multiple Immune Checkpoint Molecules within the Tumor Microenvironment,” J Immunol 200(1), 347-354.
19. Parra, E. R. et al. (2017) “Validation of Multiplex Immunofluorescence Panels Using Multispectral Microscopy for Immune-Profiling of Formalin-Fixed and Paraffin-Embedded Human Tumor Tissues,” Sci Rep 7(1), 13380.
20. Tsurui, H. et al. (2000) “Seven-Color Fluorescence Imaging of Tissue Samples Based on Fourier Spectroscopy and Singular Value Decomposition,” J Histochem Cytochem 48(5), 653-662.
21. Gerdes, M. J. et al. (2013) “Highly Multiplexed Single-Cell Analysis of Formalin-Fixed, Paraffin-Embedded Cancer Tissue,” Proc Natl Acad Sci USA 110(29), 11982-11987.
22. Schubert, W. et al. (2006) “Analyzing Proteome Topology and Function by Automated Multidimensional Fluorescence Microscopy,” Nat Biotechnol 24(10), 1270-1278.
23. Wahlby, C. et al. (2002) “Sequential Immunofluorescence Staining and Image Analysis for Detection of Large Numbers of Antigens in Individual Cell Nuclei,” Cytometry 47(1), 32-41.
24. Giesen, C. et al. (2014) “Highly Multiplexed Imaging of Tumor Tissues with Subcellular Resolution by Mass Cytometry,” Nat Methods 11(4), 417-422.
25. Black, S. et al. (2021) “Codex Multiplexed Tissue Imaging with DNA-Conjugated Antibodies,” Nat. Protoc. 16(8), 3802-3835.
26. Arentz, G. et al. (2017) “Applications of Mass Spectrometry Imaging to Cancer,” Adv Cancer Res 134, 27-66.
27. Caprioli, R. M. et al. (1997) “Molecular Imaging of Biological Samples: Localization of Peptides and Proteins Using MALDI-Tof MS,” Anal Chem 69(23), 4751-4760.
28. Buchberger, A. R. et al. (2018) “Mass Spectrometry Imaging: A Review of Emerging Advancements and Future Insights,” Anal Chem 90(1), 240-265.
29. Takats, Z. et al. (2004) “Mass Spectrometry Sampling under Ambient Conditions with Desorption Electrospray Ionization,” Science 306(5695), 471-473.
30. Zavalin, A. et al. (2012) “Direct Imaging of Single Cells and Tissue at Sub-Cellular Spatial Resolution Using Transmission Geometry MALDI MS,” J Mass Spectrom 47(11), i.
31. Kompauer, M. et al. (2017) “Atmospheric Pressure MALDI Mass Spectrometry Imaging of Tissues and Cells at 1.4-Mum Lateral Resolution,” Nat Methods 14(1), 90-96.
32. Korber, A. et al. (2022) “Fast Mass Microscopy: Mass Spectrometry Imaging of a Gigapixel Image in 34 Minutes,” Anal. Chem. 94(42), 14652-14658.
33. Piehowski, P. D. et al. (2020) “Automated Mass Spectrometry Imaging of over 2000 Proteins from Tissue Sections at 100-Mum Spatial Resolution,” Nat. Commun. 11(1), 8.
34. Thiery, G. et al. (2007) “Multiplex Target Protein Imaging in Tissue Sections by Mass Spectrometry—Tamsim,” Rapid Commun. Mass Spectrom. 21(6), 823-829.
35. Thiery, G. et al. (2008) “Improvements of Targeted Multiplex Mass Spectrometry Imaging,” Proteomics 8(18), 3725-3734.
36. Yao, J. et al. (1998) “Importance of Matrix:Analyte Ratio for Buffer Tolerance Using 2,5-Dihydroxybenzoic Acid as a Matrix in Matrix-Assisted Laser Desorption/Ionization-Fourier Transform Mass Spectrometry and Matrix-Assisted Laser Desorption/Ionization-Time of Flight,” J Am Soc Mass Spectrom 9(8), 805-813.
37. Duenas, M. E. et al. (2016) “Matrix Recrystallization for MALDI-MS Imaging of Maize Lipids at High-Spatial Resolution,” J Am Soc Mass Spectrom 27(9), 1575-1578.
38. Lemaire, R. et al. (2007) “Tag-Mass: Specific Molecular Imaging of Transcriptome and Proteome by Mass Spectrometry Based on Photocleavable Tag,” J Proteome Res 6(6), 2057-2067.
39. El Ayed, M. et al. (2010) “MALDI Imaging Mass Spectrometry in Ovarian Cancer for Tracking, Identifying, and Validating Biomarkers,” Med Sci Monit 16(8), BR233-245.
40. Franck, J. et al. (2009) “MALDI Imaging Mass Spectrometry: State of the Art Technology in Clinical Proteomics,” Mol Cell Proteomics 8(9), 2023-2033.
41. Wilschefski, S. C. and Baxter, M. R. (2019) “Inductively Coupled Plasma Mass Spectrometry: Introduction to Analytical Aspects,” Clin Biochem Rev 40(3), 115-133.
42. Han, G. et al. (2018) “Metal-Isotope-Tagged Monoclonal Antibodies for High-Dimensional Mass Cytometry,” Nat. Protoc. 13(10), 2121-2148.
43. Olejnik, J. et al. (1995) “Photocleavable Biotin Derivatives: A Versatile Approach for the Isolation of Biomolecules,” Proc. Natl. Acad. Sci. USA 92, 7590-7594.
44. Enjalbal, C. et al. (2005) “MALDI-Tof MS Analysis of Soluble PEG Based Multi-Step Synthetic Reaction Mixtures with Automated Detection of Reaction Failure,” J Am Soc Mass Spectrom 16(5), 670-678.
45. McKay, C. S. and Finn, M. G. (2014) “Click Chemistry in Complex Mixtures: Bioorthogonal Bioconjugation,” Chem Biol 21(9), 1075-1101.
46. Pandori, M. W. et al. (2002) “Photochemical Control of the Infectivity of Adenoviral Vectors Using a Novel Photocleavable Biotinylation Reagent,” Chem. Biol. 9(5), 567-573.
47. Tsaneva, M. and Van Damme, E. J. M. (2020) “130 Years of Plant Lectin Research,” Glycoconj J 37(5), 533-551.
48. Nielsen, C. B. et al. (1999) “The Solution Structure of a Locked Nucleic Acid (Lna) Hybridized to DNA,” J Biomol Struct Dyn 17(2), 175-191.
49. Kishi, J. Y et al. (2019) “Saber Amplifies FISH: Enhanced Multiplexed Imaging of RNA and DNA in Cells and Tissues,” Nature Methods 16(6), 533-544.
50. Oviano, M. and Bou, G. (2019) “Matrix-Assisted Laser Desorption Ionization-Time of Flight Mass Spectrometry for the Rapid Detection of Antimicrobial Resistance Mechanisms and Beyond,” Cin MicrobiolRev 32(1).
51. (2019) “The Path of Biomolecular Mass Spectrometry into Open Research,” Nat. Commun. 10(1), 4029.
52. Li, B. et al. (2016) “A One-Step Matrix Application Method for MALDI Mass Spectrometry Imaging of Bacterial Colony Biofilms,” J Mass Spectrom 51(11), 1030-1035.
53. Menger, R. F. et al. (2015) “MALDI Mass Spectrometric Imaging of the Nematode Caenorhabditis Elegans,” Current Metabolomics 3(2), 130-137.
54. Lanni, E. J. et al. (2012) “Mass Spectrometry Imaging and Profiling of Single Cells,” J Proteomics 75(16), 5036-5051.
55. Niehaus, M. et al. (2019) “Transmission-Mode MALDI-2 Mass Spectrometry Imaging of Cells and Tissues at Subcellular Resolution,” Nat Methods 16(9), 925-931.
56. Pantel, K. and Alix-Panabieres, C. (2010) “Circulating Tumour Cells in Cancer Patients: Challenges and Perspectives,” Trends Mol. Med. 16(9), 398-406.
57. Maheswaran, S. and Haber, D. A. (2010) “Circulating Tumor Cells: A Window into Cancer Biology and Metastasis,” Curr Opin Genet Dev 20(1), 96-99.
58. Hwang, W. L. et al. (2018) “Molecular Analysis of Circulating Tumors Cells: Biomarkers Beyond Enumeration,” Adv Drug Deliv Rev 125, 122-131.
59. Varkey, J. and Nicolaides, T. (2021) “Tumor-Educated Platelets: A Review of Current and Potential Applications in Solid Tumors,” Cureus 13(11), e19189.
60. Lin, D. et al. (2021) “Circulating Tumor Cells: Biology and Clinical Significance,” Signal Transduct Target Ther 6(1), 404.
61. Li, J. et al. (2018) “Clinical Applications of Liquid Biopsy as Prognostic and Predictive Biomarkers in Hepatocellular Carcinoma: Circulating Tumor Cells and Circulating Tumor DNA,” J. Exp. Clin. Cancer Res. 37(1), 213.
62. Toss, A. et al. (2014) “Ctc Enumeration and Characterization: Moving toward Personalized Medicine,” Ann Transl Med 2(11), 108.
63. Ding, Z. et al. (2022) “Proteomics Technologies for Cancer Liquid Biopsies,” Mol. Cancer 21(1), 53.
64. Yalcin, E. B. and de la Monte, S. M. (2015) “Review of Matrix-Assisted Laser Desorption Ionization-Imaging Mass Spectrometry for Lipid Biochemical Histopathology,” J Histochem Cytochem 63(10), 762-771.
65. Angel, P. M. et al. (2017) “Advances in MALDI Imaging Mass Spectrometry of Proteins in Cardiac Tissue, Including the Heart Valve,” Biochim Biophys Acta Proteins Proteom 1865(7), 927-935.
66. Hankin, J. A. et al. (2007) “Sublimation as a Method of Matrix Application for Mass Spectrometric Imaging,” J Am Soc Mass Spectrom 18(9), 1646-1652.
67. Kulkarni, P. et al. (2018) “Laesi Mass Spectrometry Imaging as a Tool to Differentiate the Root Metabolome of Native and Range-Expanding Plant Species,” Planta 248(6), 1515-1523.
68. Olejnik, J. et al. (1999) “Photocleavable Peptide-DNA Conjugates: Synthesis and Applications to DNA Analysis Using MALDI-MS,” Nucleic Acids Res 27(23), 4626-4631.
69. Nabiev, I. R. et al. (1990) “Surface-Enhanced Raman Spectroscopy of Biomolecules. Part II. Application of Short- and Long-Range Components of Seers to the Study of the Structure and Function of Membrane Proteins.,” J. Raman Spectrosc. 21(1).
70. Asher, I. M. et al. (1974) “Raman Spectroscopic Study of the Valinomycin—Kscn Complex,” J. Mol. Biol. 89(1), 205-222.
71. Rothschild, K. J. and Stanley, H. E. (1974) “Raman Spectroscopic Investigation of Gramicidin a' Conformations,” Science 185(4151), 616-618.
72. Cao, Y C. et al. (2003) “Raman Dye-Labeled Nanoparticle Probes for Proteins,” J. Am. Chem. Soc. 125(48), 14676-14677.
73. Wang, Y and Irudayaraj, J. (2013) “Surface-Enhanced Raman Spectroscopy at Single-Molecule Scale and Its Implications in Biology,” Phil. Trans. R. Soc. B 368(1611), 20120026.
74. Zhu, A. et al. (2023) “Advances in Surface-Enhanced Raman Spectroscopy Technology for Detection of Foodborne Pathogens,” Compr Rev Food Sci Food Saf22(3), 1466-1494.
75. Argade, P. V. et al. (1981) “Resonance Raman Spectroscopy of Specifically [Epsilon-15n]Lysine-Labeled Bacteriorhodopsin,” Proc. Natl. Acad. Sci. U.S.A 78(3), 1643-1646.
76. Rothschild, K. J. et al. (1982) “The Site of Attachment of Retinal in Bacteriorhodopsin. A Resonance Raman Study,” J. Biol. Chem. 257(15), 8592-8595.
77. Kim, J. H. et al. (2011) “Encoding Peptide Sequences with Surface-Enhanced Raman Spectroscopic Nanoparticles,” Chem. Commun. (Camb.) 47(8), 2306-2308.
78. Lee, H. J. et al. (2019) “Electronic Preresonance Stimulated Raman Scattering Imaging of Red-Shifted Proteorhodopsins: Toward Quantitation of the Membrane Potential,” J Phys Chem Lett 10(15), 4374-4381.
79. Angelo, M. et al. (2014) “Multiplexed Ion Beam Imaging of Human Breast Tumors,” Nat. Med. 20(4), 436-442.
80. Kuett, L. et al. (2022) “Three-Dimensional Imaging Mass Cytometry for Highly Multiplexed Molecular and Cellular Mapping of Tissues and the Tumor Microenvironment,” Nature Cancer 3(1), 122-133.
81. Hickey, J. W. et al. (2022) “Spatial Mapping of Protein Composition and Tissue Organization: A Primer for Multiplexed Antibody-Based Imaging,” Nature Methods 19(3), 284-295.
82. Lin, J. R. et al. (2018) “Highly Multiplexed Immunofluorescence Imaging of Human Tissues and Tumors Using T-Cycif and Conventional Optical Microscopes,” eLife 7.
83. Goltsev, Y et al. (2018) “Deep Profiling of Mouse Splenic Architecture with Codex Multiplexed Imaging,” Cell 174(4), 968-981 e915.
84. Neumann, E. K. et al. (2021) “Protocol for Multimodal Analysis of Human Kidney Tissue by Imaging Mass Spectrometry and Codex Multiplexed Immunofluorescence,” STAR Protocols 2(3), 100747.
85. (2012) in Evolution of Translational Omics: Lessons Learned and the Path Forward (Micheel, C. M., et al., Eds.), Washington (DC).
86. Zupancic, D. et al. (2020) “Combined Lectin- and Immuno-Histochemistry (Clih) for Applications in Cell Biology and Cancer Diagnosis: Analysis of Human Urothelial Carcinomas,” Eur. J. Histochem. 64(3).
87. Siuzdak, G. (2004) “Cover Feature: Book Preview: An Introduction to Mass Spectrometry Ionization: An Excerpt from the Expanding Role of Mass Spectrometry in Biotechnology, 2nd Ed.; Mcc Press: San Diego, 2005,” JALA 9(2), 50-63.
88. Wisztorski, M. et al. (2010) “MALDI Direct Analysis and Imaging of Frozen Versus Ffpe Tissues: What Strategy for Which Sample?,” Methods Mol Biol 656, 303-322.
89. Gundry, R. L. et al. (2009) “Preparation of Proteins and Peptides for Mass Spectrometry Analysis in a Bottom-up Proteomics Workflow,” Curr Protoc Mol Biol Chapter 10, Unit10 25.
90. Barrett, A. S. et al. (2017) “Hydroxylamine Chemical Digestion for Insoluble Extracellular Matrix Characterization,” J. Proteome Res. 16(11), 4177-4184.
91. Drake, R. R. et al. (2018) “In Situ Imaging of N-Glycans by MALDI Imaging Mass Spectrometry of Fresh or Formalin-Fixed Paraffin-Embedded Tissue,” Curr Protoc Protein Sci 94(1), e68.
92. Angel, P. M. et al. (2019) “Extracellular Matrix Imaging of Breast Tissue Pathologies by MALDI-Imaging Mass Spectrometry,” Proteomics Clinical Applications 13(1), e1700152.
93. Lazova, R. et al. (2020) “Histopathology-Guided Mass Spectrometry Differentiates Benign Nevi from Malignant Melanoma,” J. Cutan. Pathol. 47(3), 226-240.
94. Behrendt, R. et al. (2016) “Advances in Fmoc Solid-Phase Peptide Synthesis,” J Pept Sci 22(1), 4-27.
95. Stawikowski, M. and Fields, G. B. (2012) “Introduction to Peptide Synthesis,” Curr Protoc Protein Sci Chapter 18, Unit 18 11.
96. Morpurgo, M. et al. (1999) “N-Hydroxysuccinimide Carbonates and Carbamates Are Useful Reactive Reagents for Coupling Ligands to Lysines on Proteins,” J Biochem Biophys Methods 38(1), 17-28.
97. Uhlen, M. et al. (2015) “Proteomics. Tissue-Based Map of the Human Proteome,” Science 347(6220), 1260419.
98. Wang, F. et al. (2012) “Rnascope: A Novel in Situ RNA Analysis Platform for Formalin-Fixed, Paraffin-Embedded Tissues,” Journal ofMolecular Diagnostics 14(1), 22-29.
99. Saka, S. K. et al. (2019) “Immuno-Saber Enables Highly Multiplexed and Amplified Protein Imaging in Tissues,” Nat. Biotechnol. 37(9), 1080-1090.

We claim:

1. A method of treating a tissue sample, comprising:
a) providing i) a mass spectrometric imaging instrument, ii) a source of UV light exogenous to said mass spectrometric imaging instrument, iii) a tissue sample and iv) a first solution of one or more probes conjugated to photocleavable mass-tags;
b) treating said tissue sample with said first solution of one or more probes to create a probed tissue sample;
c) treating the probed tissue sample with a fixative so that said probes are fixed to said tissue sample;
d) illuminating said fixed probes with said exogenous source of UV light so as to photocleave at least a portion of said mass-tags; and
e) detecting, using said mass spectrometric imaging instrument, said mass-tags, or fragments thereof as molecular ions, from at least one of said fixed probes.

2. The method of claim 1, wherein said tissue sample is mounted on a slide.

3. The method of claim 2, wherein said slide is coated with Poly-L-lysine to promote adhesion of said tissue sample to said slide.

4. The method of claim 2, wherein said tissue sample is formalin-fixed and paraffin-embedded.

5. The method of claim 2, wherein said first solution of one or more probes in step b) is directly overlaid onto said tissue sample.

6. The method of claim 1, wherein said fixative is paraformaldehyde.

7. The method of claim 1, further comprising a wash step after step b).

8. The method of claim 1, further comprising a wash step after step c)

9. The method of claim 1, wherein said first solution comprises a plurality of different probes, each different probe conjugated to a unique photocleavable mass-tag, and at least one of said probes targeting a known biomarker in said tissue sample.

10. The method of claim 9, wherein said plurality of different probes are antibodies conjugated to photocleavable mass tags.

11. The method of claim 10, wherein said antibodies are in a mixture.

12. The method of claim 1, wherein said mass-tags comprise a plurality of amino acids.

13. The method of claim 4, wherein the tissue sample is subjected to a treatment prior to step b), said treatment comprising deparaffinization.

14. The method of claim 13, wherein said deparaffinization is performed with xylene.

15. The method of claim 13, wherein the tissue sample is further subjected to a treatment, said treatment comprising rehydration.

16. The method of claim 15, wherein said rehydration is performed with a series of ethanol/water mixtures and aqueous saline buffers.

17. The method of claim 15, wherein the tissue sample is further subjected to a treatment, said treatment comprising antigen retrieval.

18. The method of claim 1, wherein a matrix compound is applied to said mass-tags before step e).

19. The method of claim 10, wherein said plurality of different antibody probes are dual-labeled antibody probes, each of said different dual-labeled probes reactive with a different target and conjugated to a photocleavable mass-tag and a detectable label.

20. The method of claim 10, wherein the plurality of different antibody probes are fixed to the tissue after step c) to prevent washing out of the antibody in subsequent steps.

21. The method of claim 1, wherein said tissue sample is treated with a fixative before step b).

22. The method of claim 1, wherein said tissue sample is fresh frozen.

23. The method of claim 18, wherein the tissue sample is washed after step e) to remove said matrix compound to create a washed tissue sample.

24. The method of claim 23, further comprising f) contacting said washed tissue sample with a second solution of one or more probes conjugated to photocleavable mass-tags.

25. The method of claim 19, wherein said detectable label is an oligonucleotide tag.
